(19) United States
(12) Patent Application Publication
(10) Pub. No.: US 2022/0145355 A1
(43) Pub. Date: May 12, 2022

(54) METHODS AND KITS FOR DETERMINING CELL SECRETED BIOMOLECULES

(71) Applicant: EVORION BIOTECHNOLOGIES GMBH, Münster (DE)

(72) Inventors: Hans KLEINE-BRÜGGENEY, Münster (DE); Robert WEINGARTEN, Münster (DE); Sebastian BÜHREN, Nottuln (DE)

(21) Appl. No.: 17/436,154

(22) PCT Filed: Mar. 13, 2020
(86) PCT No.: PCT/EP2020/056975

§ 371 Date: Sep. 3, 2021

(30) Foreign Application Priority Data
Mar. 13, 2019 (EP) 19162691.0

(51) Int. Cl. C12Q 1/6804; C12Q 1/6876; C12M 1/32; C12M 1/00; C12M 1/12; C12M 1/34; G01N 33/543

(52) U.S. Cl. CPC C12Q 1/6804; C12Q 1/6876; C12M 23/12; G01N 33/54306; C12M 25/14; C12M 41/00; C12M 23/34

(57) ABSTRACT

The invention inter alia pertains to methods and kits for analysis of one or more cell released biomolecules. Furthermore, the invention relates to a plurality of sequenceable products comprising different sequence elements. The described technology is useful for a variety of applications, in particular biomolecule analysis applications, e.g. for obtaining biomolecule release profiles of single cells in a multiplexed manner, wherein the cells are provided in a matrix

### BACKGROUND OF THE INVENTION

Cells have been found to be phenotypically different, despite being genetically identical. Indeed, cell populations of seemingly same cells appear to always contain cells which are different from each other at a particular resolution of inspection (Alterschule and Wu, 2010, Cell, 141(4), pages 559-563). Standard assays are only capable of determining average responses of the cells, which is interpreted as the response of all cells in that sample. Specialized cells which exist in nearly all cell populations (e.g. cancer stem cells) are ignored in such bulk assays and valuable information about these cells is lost. Yet, the knowledge about individual cells is important to elucidate molecular mechanisms, which are for instance involved in cancer formation, ageing and immune responses. In order to understand and utilize the intercellular differences, for instance, for therapeutic purposes, there is a need for analysing cells on a single-cell level. Importantly, at a single-cell level, methods and techniques are required, which allow analysis of large numbers of single-cells in order to screen sufficiently large cell populations. Furthermore, these methods and techniques need to allow analysis of multiple phenotypic traits throughout the cellular cultivation. In addition, a sequential coupling of the determined phenotypic traits with genomic data is desirable.

With the aim to analyse molecules of single cells or small cell populations, an immunoassay, called FluoroSpot assay, has been developed (Janetzki et al., 2014, Cells, 2, pages 1102-1115). Therefore, molecule-specific capture antibodies (e.g. against cytokines) are added to an assay plate having wells. Afterwards, cells are added to the wells in suspension at a low dilution and the assay plate is incubated to allow for cytokine secretion by the cells. The secreted cytokines may be captured by the immobilized cytokine-specific capture antibodies at the bottom of wells. Thereafter, the cells may be removed and the wells may be washed and added with molecule-specific detection antibodies and fluorophore conjugates. Finally, secretory footprints (or spots) of the secreted molecules (e.g. cytokines) are captured by way of imaging, and such images are analysed for identifying multiple analytes, counting number of cells secreting the analytes, and the like. Another end point analysis of single cells has been developed to predict the response of cells to anti-PD-1 immunotherapy by single-cell mass cytometry. Therefore, metal labelled antibodies are used, which bind to cellular proteins, and analysed via time of flight mass spectrometry. A sequential data analysis sorts the detected signals for each cell, while the number of bound and labelled antibodies enables a quantitative analysis (Krieg et al., 2018, Nature Medicine, 24, pages 144-153).

In order to analyse multiple molecules secreted by single cells throughout the cultivation and acquire time-resolved information about the secreted molecules, Lu et al. developed a microfabricated device based fluorescence-barcoding technique (Lu et al., 2015, Proc. Natl. Acad. Sci. USA, 112(7), pages E607-E615): Statistically distributed, mostly single cells are captured in small PDMS compartments, which are then covered by an antibody-coated array. Similar to a barcode, antibodies are placed as fine lines onto the array, wherein one line corresponds to one type of antibody specific against a particular molecule (here cytokine). Throughout the cultivation, the single cells secrete cytokines, which are then captured by the specific antibody. After the cultivation time (e.g. 24 h), the array is removed and a sandwich assay is performed. This assay applies fluorescently labelled detection antibodies to the array, which bind to a different epitope of the bound target cytokines for staining (up to 3 colours). Afterwards, imaging can be conducted, which reveals in case of a secreted cytokine a coloured spot, whereas no colour can be detected when the cytokine is not detected. The advantage of this technology is a multiplexed analysis of statistically-distributed single cells. Moreover, up to 12,000 PDMS compartments can be present in one microfabricated device, allowing a highly multiplexed analysis (Xue et al., 2017, Journal for ImmunoTherapy of Cancer, 5(1):85). Yet, there is a need for methods that improve analysis of multiple molecules secreted by single cells.

Moreover, it is desirable to study the interaction of two or more cells or cell types in a defined manner. For instance, priming immune cells against certain cancer cells has proven to be an effective strategy in inhibiting cancer propagation and is envisioned in form of cancer immunotherapy as the next step in cancer treatment (Steer et al., 2010, Oncogene, 29, pages 6301-6313). Therefore, methods and techniques are required, which enable to place a defined number of (different) cells next to each other in a closed compartment. Furthermore, interfaces should be present that allow integration of methods for multiplexed analysis of secreted molecules to study the cellular interaction. Noteworthy, analysis of single cells or cellular interactions should allow transferring the gained insights to in vivo conditions. This requires establishing an environment for the cells which mimics the conditions the cells encounter in the body.

The invention aims at avoiding drawbacks of the prior art methods. In particular, it is an object to analyse biomolecules secreted by at least one cell, in some cases exactly one cell, in a multiplex fashion. It is an object to analyse multiple molecules which are secreted by single cells in a time-dependent manner. It is another object, to perform dynamic studies of living single cells and small populations of cells which can increase the understanding of the interconnecting molecular events coupling phenotypic events to the underlying genotype of particular cells. It is another object to provide a microenvironment to the cells that mimics the conditions the cells encounter in vivo.

### SUMMARY OF THE INVENTION

The present invention relates to the analysis of cell-released biomolecules. In comparison to the prior art, the present invention allows analysing multiple biomolecules of interest in a time-dependent manner, wherein the cells that release biomolecules are provided in an environment that is capable of mimicking the native cell environment.

According to a first aspect, the present disclosure provides a method for analyzing one or more cell released biomolecules, comprising providing a cell-laden matrix, wherein the cell-laden matrix comprises at least one cell that releases one or more biomolecules of interest, wherein the method comprises the following steps:
a) providing a capture matrix, wherein the capture matrix comprises one or more types of capture molecules, wherein each type of capture molecule binds a biomolecule of interest;
b) incubating the cell-laden matrix to allow release of the one or more biomolecules of interest and binding the one or more biomolecules of interest to the one or more types of capture molecules of the capture matrix;
c) adding one or more types of detection molecules, wherein each type of detection molecule specifically binds a biomolecule of interest, and wherein each type of detection molecule comprises a barcode label which comprises a barcode sequence (B_{S}) indicating the specificity of the detection molecule;
d) generating a sequenceable reaction product which comprises at least
   (i) the barcode sequence (B_{S}), and
   (ii) a barcode sequence (B_{T}) for indicating a time information, and/or
   (iii) a barcode sequence (B_{P}) for indicating a position information, and
   (iv) optionally a unique molecular identifier (UMI) sequence,

wherein generation of the sequenceable reaction product comprises the use of
at least one oligonucleotide, optionally a primer, that is capable of hybridizing to the barcode label of the at least one type of detection molecule (preferred) or
the use of at least one oligonucleotide that is ligated to the barcode label of the at least one type of detection molecule.

The method may additionally comprise e) sequencing the generated reaction product.

According to a second aspect, the present disclosure provides a kit comprising
a) one or more types of detection molecules, wherein each type of detection molecule specifically binds a biomolecule of interest, and wherein each type of detection molecule comprises a barcode label which comprises a barcode sequence (B_{S}) indicating the specificity of the detection molecule; and
b) at least one oligonucleotide, optionally a primer, that is preferably capable of hybridizing to the barcode label of the at least one type of detection molecule.

The kit may be used for performing the method according to the first aspect.

According to a third aspect, the present disclosure provides a plurality of sequenceable products, wherein each sequenceable product comprises at least the following sequence elements
(i) a barcode sequence (B_{S}) for indicating a specificity, and
(ii) a barcode sequence (B_{T}) for indicating a time information, and/or
(iii) a barcode sequence (B_{P}) for indicating a position information, and
(iv) optionally a unique molecular identifier (UMI) sequence.

According to the present invention, different types of matrices are applied, which either are loaded with at least one cell (corresponding to a cell-laden matrix) or with one or more types of capture molecules (corresponding to a capture matrix). The matrices have multiple advantages, including that cells can be cultivated under physiological conditions inside the matrix. On the other hand the capture molecules comprised in the capture matrix can be flexibly attached to the matrix which has an increased surface area for attachment (e.g. in comparison to solid particles, wherein only the surface is available for attachment). Each matrix type can then be advantageously brought into contact or proximity to each other, preferably inside a compartment. Such a compartment may be preferably provided by a microfabricated cell culture device, allowing matrices to be transported into and away from the compartment (and if desired also away from the microfabricated cell culture device into another format (e.g. well plate)). Moreover, the microfabricated cell culture device preferably comprises means to switch the compartment between an open and an isolated state throughout cultivation. For instance, in an isolated compartment, biomolecules of interest that are released by the at least one cell throughout the incubation can diffuse out of the cell-laden matrix after their release. Afterwards, the biomolecules of interest can diffuse to the capture matrix, wherein capture molecules can bind biomolecules of interest, while due to the isolated state of the compartment, the biomolecules are not lost (e.g. due to perfusion or washing). Preferably, each type of capture molecule binds a different biomolecule of interest. Hence, advantageously biomolecules of interest can be captured by the capture matrix, respectively the one or more types of capture molecules. Afterwards, detection molecules are added, wherein each type of detection molecule preferably binds to a different biomolecule of interest, and wherein the molecules of each type of detection molecule comprise a barcode label which comprises a barcode sequence (B_{S}) indicating the specificity of the detection molecule. This has the advantage, that multiple biomolecules of interest can be analyzed in a multiplexed manner, as the barcode label allows for a subsequent differentiation, e.g. by sequencing. Furthermore, the method generates a sequenceable reaction product, comprising in addition to the barcode sequence (B_{S}) a barcode sequence (B_{T}) for indicating a time information and/or a barcode sequence (B_{P}) for indicating a position information. Hence, not only the biomolecule of interest can be captured and sequentially differentiated for a multiplexed analysis based on the generated sequenceable product, but also different time points of the incubation process and/or different positions (e.g. positions which may comprise different cells or cells incubated under different conditions). In this respect, the generation of a sequenceable reaction product advantageously allows subsequent pooling of multiple reaction products for sequencing, as these can be differentiated and clearly assigned based on the comprised sequence elements/barcodes. Such differentiation preferably takes place after sequencing the generated reaction product. Hence, a very efficient and multiplexed analysis with a high throughput of cell-released biomolecules is achieved by the provided technology. The generated sequenceable reaction product can optionally comprise a unique molecule identifier (UMI) sequence allowing to analyze the bound molecules in a highly quantitative manner, which is advantageous for an absolute analysis of biomolecules of interest.

Other objects, features, advantages and aspects of the present application will become apparent to those skilled in the art from the following description and appended claims. It should be understood, however, that the following description, appended claims, and specific examples, while indicating preferred embodiments of the application, are given by way of illustration only.

### DETAILED DESCRIPTION OF THE INVENTION

The disclosed technology is now explained in further detail.

### The Method According to the First Aspect

According to a first aspect, the present disclosure provides a method for analyzing one or more cell released biomolecules, comprising providing a cell-laden matrix, wherein the cell-laden matrix comprises at least one cell that releases, e.g. secretes, one or more biomolecules of interest, wherein the method comprises the following steps:
a) providing a capture matrix, wherein the capture matrix comprises one or more types of capture molecules, wherein each type of capture molecule binds a biomolecule of interest;
b) incubating the cell-laden matrix to allow release of the one or more biomolecules of interest and binding the one or more biomolecules of interest to the one or more types of capture molecules of the capture matrix;
c) adding one or more types of detection molecules, wherein each type of detection molecule specifically binds a biomolecule of interest, and wherein each type of detection molecule comprises a barcode label which comprises a barcode sequence (B_{S}) indicating the specificity of the detection molecule;
d) generating a sequenceable reaction product which comprises at least
   (i) the barcode sequence (B_{S}), and
   (ii) a barcode sequence (B_{T}) for indicating a time information, and/or
   (iii) a barcode sequence (B_{P}) for indicating a position information, and
   (iv) optionally a unique molecular identifier (UMI) sequence,
      wherein generation of the sequenceable reaction product comprises the use of at least one oligonucleotide, optionally a primer, that is capable of hybridizing to the barcode label of the at least one type of detection molecule; and
e) preferably sequencing the generated reaction product.

In a less preferred alternative, the generation of the sequenceable product in d) comprises the use of at least one oligonucleotide that is ligated to the barcode label of the at least one type of detection molecule.

According to the first aspect of the present disclosure, a method is provided for analyzing one or more cell released biomolecules, wherein the cells are provided by a cell-laden matrix. The cell-laden matrix comprises at least one cell that is capable of releasing one or more biomolecules of interest for instance by secretion. According to one embodiment the cell-laden matrix comprises a hydrogel, wherein preferably the matrix material is provided by a hydrogel. According to a preferred embodiment, the at least one cell is encapsulated inside a matrix. A matrix preferably provides a three-dimensional matrix which can advantageously surround the at least one cell. This advantageously provides an environment to the cells that mimics the environment the cells naturally encounter and thus a more physiological environment can be established. For instance, a matrix can be provided that mimics the biochemical, mechanical and structural environment a cell would encounter in nature. In a particular embodiment, a human or human-derived cell may be encapsulated by a hydrogel matrix, which can be advantageously adapted to provide a particular three-dimensional environment to the cells, including one that the cell would encounter in the body in a vital or diseased state. Suitable embodiments for the at least one cell are described herein further below throughout the further embodiments of the method of the first aspect and it is referred thereto. The further below disclosed embodiments can be advantageously applied for the method according to the first aspect.

According to one embodiment, the cell-laden matrix comprises at least one cell. The cell-laden matrix may advantageously comprise a pre-defined cell composition. Such a pre-defined cell composition can be selected from the group comprising a single cell, multiple cells, cell colonies, mini-tissues, mini-organs, tissue samples, and combinations thereof. Other cell compositions (i.e. cell/cells to be provided in form of a cell-laden matrix) are described further below and also apply here. The pre-defined cell composition advantageously enables profiling of secreted molecules from pre-defined cell compositions (arrangements). One result of such an embodiment may be that only secretomes (e.g. the sum of released biomolecules of interest) from cells of interest will be quantified. The embodiment advantageously enables to customize experiments in a cost-effective manner maintaining high data integrity.

The released biomolecules of interest according to the present disclosure can be a number of different kinds/types of biomolecules. Various biomolecules which may be analyzed in scope of the present invention are disclosed below in the section disclosing the further embodiments of the method of the first aspect and these embodiments also apply here. Particular biomolecules of interest are proteins which may be released by the at least one cell in scope of secretion processes. Exemplary proteins may be cytokines which are secreted by cells and their analysis allows to study the interaction of cells in view of cell-cell communication by cytokines. Such an analysis is important in understanding cellular processes and can contribute to study for instance the interaction between cancer and immune cells to improve immuno-therapies. Various other applications of the present disclosure are feasible and are apparent throughout the present disclosure.

Moreover, the matrices disclosed herein, including the cell-laden matrix and capture matrix, preferably comprise a hydrogel, which may be formed upon the gelation/polymerization/curing of a monomer, pre-polymer, precursor, polymer and/or building block. Particular, monomers, pre-polymers, precursors, polymers and/or building blocks are disclosed below in the further embodiments of the method of the first aspect and can be advantageously applied in order to form matrices of the present disclosure. Suitable embodiments are described herein.

In addition, the matrices disclosed herein, including the cell-laden matrix and capture matrix, may have different shapes. In a preferred embodiment, matrices are formed using droplet microfluidics. For example, a flow focusing geometry can be used for the generation of highly monodisperse droplets having a spherical shape. If the droplet diameter is larger than the width/height of the microfluidic channel in which the hydrogel formation may occur, formed matrices have a plug-like shape. In addition, matrices may be formed by conventional pipetting. Thus, matrix solutions comprising monomers, pre-polymers, precursors, polymer and/or building blocks for gelation/polymerization/curing reactions may be pipetted on a 2D surface resulting in the formation of a droplet having the shape of a spherical segment and/or a hemi-spherical shape. The shape depends on the surface tension between the droplet and the surrounding surfaces and may be adjusted by changing the surface characteristics. In another embodiment, matrix solutions comprising monomers, pre-polymers, precursors, polymer and/or building blocks for gelation/polymerization/curing reactions may be pipetted into a geometry having a pre-defined shape (e.g. a cylindrical geometry). Thus, matrices may assume the shape of the container containing the matrix solution during matrix formation.

According to one embodiment, the volume of matrices disclosed herein, including the cell-laden matrix and capture matrix, may vary depending on the used method for matrix formation. In a preferred embodiment, matrices are formed using droplet microfluidics as described in the present disclosure having a volume within the range of 50 fl to 50 nl, in particular between 200 pl and 400 pl. In one embodiment, matrices may be formed by methods such as conventional pipetting having a volume between 0.5 μl to 500 μl, such as 1 μl to 200 μl or 2 μl to 100 μl. In one embodiment, the volume of a matrix is ≤200 μl, such as ≤100 μl, ≤50 μl, ≤10 μl, ≤1 μl, ≤0.5 μl, ≤300 nl, ≤200 nl, ≤100 nl, ≤50 nl or ≤5 nl, preferably 0.05 pl to 2000 pl;

A microfabricated cell culture device as used herein in particular refers to a device having geometries/structures with size dimensions smaller than 1000 μm while being compatible with the incubation of cells. Said geometries may be fabricated using conventional microfabrication techniques such as lithography, soft lithography, replica molding or techniques such as 3D printing, CNC-milling or injection molding.

According to one embodiment, the matrix of the cell-laden matrix (and/or the capture matrix) is a hydrogel which has one or more of the following characteristics:
a) the hydrogel comprises cross-linked hydrogel precursor molecules of the same type or of different types;
b) the hydrogel is composed of at least two different polymers with different structures as hydrogel precursor molecules, wherein optionally, at least one polymer is a copolymer;
c) the hydrogel is formed using at least one polymer which has a linear structure and at least one polymer which has a multiarm or star-shaped structure;
d) the hydrogel is formed using a t least one polymer of formula (P1)

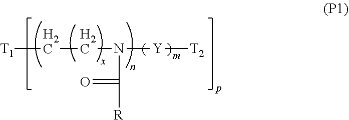

wherein
R is independently selected from a hydrogen atom, a hydrocarbon with 1-18 carbonatoms (preferably CH₃, —C₂H₅,), a C₁-C₂₅-hydrocarbon with at least one hydroxy group, a C₁-C₂₅-hydrocarbon with at least one carboxy group, (C₂-C₆)alkylthiol, (C₂-C₆)alkylamine, protected (C₂-C₆)alkylamine (preferably-(CH₂)₂₋₆—NH—CO—R (with R=tert-Butyl, perfluoroalkyl)), (C₂-C₆)alkylazide, polyethylene glycol, polylactic acid, polyglycolic acid, polyoxazoline, or wherein R is a residue R⁴
Y is a moiety containing at least one graft, comprising at least one residue R⁴,
T₁ is a terminating moiety, which may contain a residue R⁴,
T₂ is a terminating moiety, which contains a residue R⁴,
p is an integer from 1 to 10,
n is an integer greater than 1 and preferably, below 500,
m is zero or an integer of at least, preferably greater than 1, and preferably, below 500,
the sum n+m is greater than 10,
x is independently 1, 2 or 3, preferably x is independently 1 or 2, most preferably x is 1,
R⁴ independently comprise at least one functional group
   for crosslinking and/or
   for binding biologically active compounds, and
   optionally comprising a (preferably degradable) spacer moiety connecting said functional group with the binding site to the respective moiety of the structure of formula (P1),
   wherein the entirety of all m-fold and n-fold repeating units are distributed in any order within the polymer chain and wherein optionally, the polymer is a random copolymer or a block copolymer.

According to one embodiment, the matrix of the cell-laden matrix and/or the capture matrix is a particle, preferably a spherical particle. According to one embodiment, the matrices disclosed herein are preferably spherical, e.g. spherical hydrogel matrices but other forms may also be applied. Applicable shapes/forms of the matrix (such as a hemi-spherical or plaque-like shape) are described further below and also apply here. According to one embodiment, the matrix has a diameter of ≤1000 μm, such as ≤800 μm, ≤600 μm or ≤400 μm, preferably ≤200 μm, such as 5 μm to 150 μm. Other applicable diameters of the matrix are described below when disclosing the further embodiments of the method of the first aspect. According to a particular embodiment, the cell-laden matrix may be a hydrogel matrix that provides a three-dimensional environment to the at least one cell, wherein preferably the matrix is at least 5 μm and ≤200 μm in diameter.

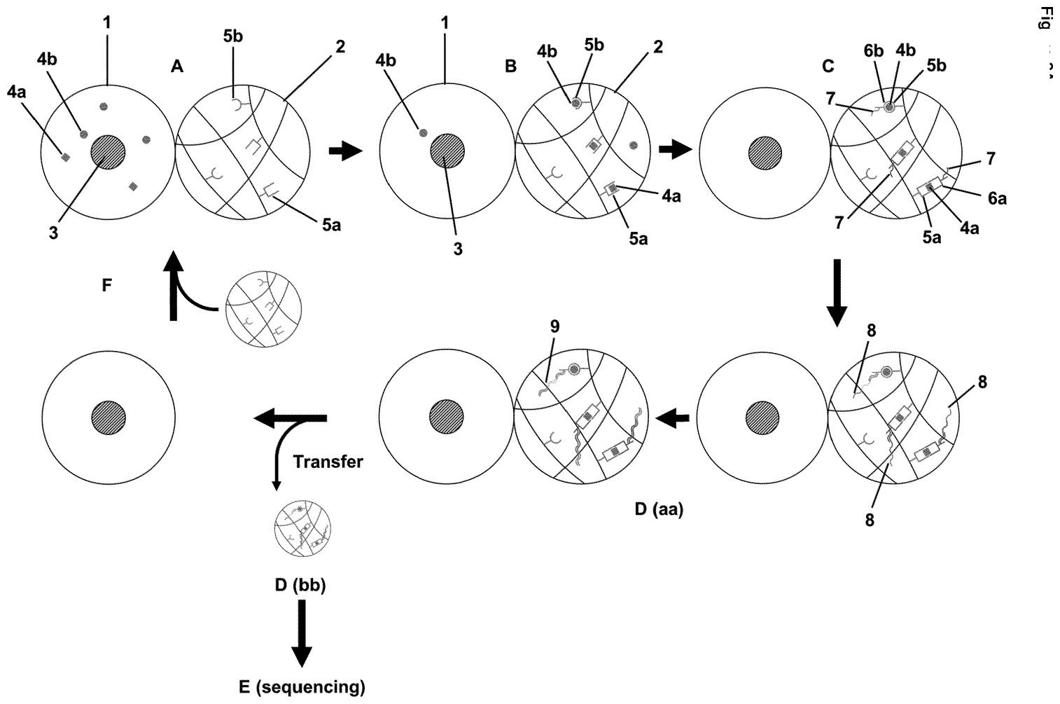

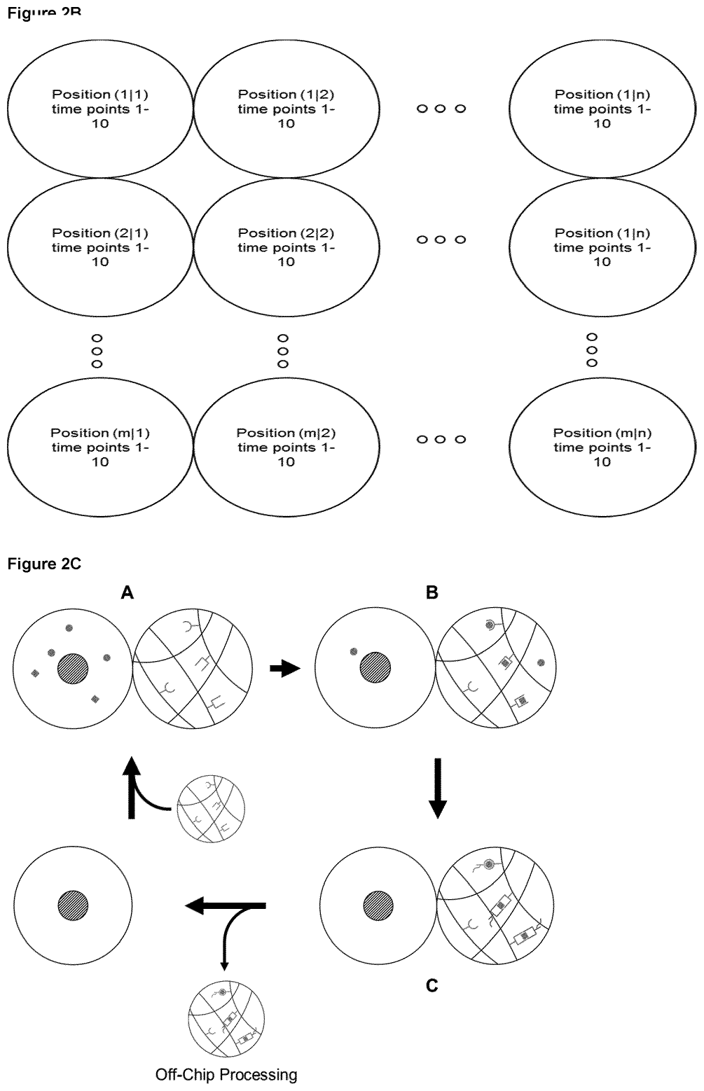

The individual steps of the method according to the first aspect will be further explained below. It is also referred to FIG. 2 which schematically illustrates exemplary embodiments of the method according to the first aspect of the present disclosure.

Steps a) and b)

In the method, a cell-laden matrix is provided. In step a), a capture matrix is provided, wherein the capture matrix comprises one or more types of capture molecules, wherein each type of capture molecule binds a biomolecule of interest. The cell-laden matrix may be provided such as e.g. prepared using methods disclosed herein.

According to step b), the cell-laden matrix is incubated to allow release of the one or more biomolecules of interest. As is disclosed herein, incubating the cell-laden matrix to allow release (e.g. secretion) of the biomolecules of interest may already occur for a time period before the capture matrix is provided in proximity to the cell-laden matrix in order to allow capture of the released biomolecules of interest by the capture matrix. However, the capture matrix may also be present during the entire incubation process. The one or more biomolecules of interest may diffuse from the cell-laden matrix to the capture matrix, where the one or more biomolecules of interest are specifically bound by the one or more types of capture molecules.

The provided capture matrix preferably comprises a hydrogel, wherein the matrix material is preferably provided by a hydrogel. According to one embodiment, the matrix is three-dimensional. According to a preferred embodiment, the capture matrix comprises a three-dimensional hydrogel. By providing a capture matrix comprising one or more types of capture molecules, a high surface area is provided for attaching the capture molecules, which advantageously allows to provide at least one type of capture molecules at a high number (e.g. in comparison to solid particles, which merely provide the surface of the particle for attaching capture molecules thereto). As disclosed above for the cell-laden matrix, the capture matrix may be formed upon gelation/polymerization/curing of a monomer, pre-polymer, precursor, polymer and/or building block, which are disclosed below in the further embodiments of the method of the first aspect and can be advantageously applied in order to form matrices of the present disclosure. The capture matrix may comprise a crosslinked monomer, pre-polymer, precursor, polymer and/or building block, known from the prior art by the skilled person. Typical polymers of the prior art may be applied, selected from the non-limiting list comprising polyacrylamide (PMA), poly(lactic acid) (PLA), poly(vinyl alcohol) (PVA), polyethylene glycol (PEG), polyoxazoline (POx), and polystyrene (PS). The capture matrix may be formed upon reaction of the same monomer, pre-polymer, precursor, polymer and/or building block or different monomer, pre-polymer, precursor, polymer and/or building block. According to a preferred embodiment, the capture matrix comprises a pore size that allows for the diffusion of at least a portion of the released biomolecules of interest into the matrix. This advantageously allows biomolecules of interest not only to access the surface but also the interior in order to bind to the one or more types of capture molecules.

According to one embodiment, the matrix is a particle, preferably a spherical particle such as a bead. According to one embodiment, the capture matrix is provided in form of a hydrogel matrix, preferably a spherical hydrogel matrix. However the form/shape of the capture matrix is not limited to a spherical shape and other shapes (such as a hemi-spherical or plaque-like shape) are also possible and described below. According to one embodiment, the matrix has a diameter of ≤1000 μm, such as ≤800 μm, ≤600 μm or ≤400 μm, preferably ≤200 μm such as 5 μm to 150 μm. Other applicable diameters of the matrix are described below and also apply here.

According to one embodiment, the capture molecules are attached to the capture matrix in order to be capable of binding a biomolecule of interest and thereby capture the biomolecule of interest in a form which can be freely moved/transported (e.g. the capture matrix can be freely transferred away from the cell-laden matrix). According to an advantageous embodiment, the capture matrix comprises at least one type of capture molecules, which can be in an exemplary embodiment at least one type of antibody with a defined specificity for a biomolecule of interest. Biomolecules of interest that are released by the at least one cell can be immobilized within the capture matrix of the present disclosure, which enables single- and multiplexing within one experiment in a highly customizable manner, as the capture molecules specifically bind to biomolecules of interest derived/released from the cell-laden matrix. Applicable capture molecules are also disclosed below in conjunction with the further embodiments of the method according to the first aspect. Exemplary capture molecules include but are not limited to antibodies, antibody fragments, aptamers, etc. One preferred capture molecule may be a capture molecule derived from an antibody. According to a preferred embodiment, one type of capture molecules comprises multiple capture molecules of the same type. Therefore, advantageously multiple biomolecules of interest of the same type can be captured, e.g. in order to perform quantitative analysis.

According to one embodiment, the capture matrix comprising the one or more types of capture molecules is positioned after a pre-defined cultivation/stimulation period. The capture matrix may also be provided together with the cell-laden matrix or shortly afterwards, in some embodiments even before the cell-laden matrix. In an embodiment, wherein the different matrices are provided shortly after each other, preferably first the cell-laden matrix is provided and then the capture matrix, so that the released biomolecules of interest can be directly bound by the one or more types of capture molecules of the capture matrix. Therefore, biomolecules of interest are first released by the at least one cell (e.g. over an incubation period) and diffuse out of the cell-laden matrix to the capture matrix. Accordingly, the matrix material of the cell-laden matrix and the capture matrix are preferably selected such that the biomolecules of interest can diffuse through the matrices and preferably do not non-specifically interact with/bind to the matrix material, so that the biomolecules of interest can be efficiently captured by the one or more types of capture molecules. Hence, it is advantageous to limit the binding of biomolecules of interest to specific binding to the capture molecules (while a certain degree of unspecific binding may occur but is not preferred). After exiting the cell-laden matrix, the biomolecules of interest diffuse to and preferably into the capture matrix, wherein the one or more biomolecules of interest are specifically bound by the one or more types of capture molecules. The capture matrix is preferably tailored such that the one or more biomolecules of interest can diffuse into the matrix and do not (or non-significantly) non-specifically interact with/bind to the matrix material. Preferably, the one or more biomolecules of interest specifically interact with the capture molecules.

Throughout incubation, artificial “capture effects” can be avoided by the present method, as released biomolecules of interest need to first diffuse out of the cell-laden matrix and then diffuse into the capture matrix to be bound by the at least one type of capture molecules. Typically, the biomolecules also need to diffuse through a part of the surrounding fluid before diffusing onto/into the capture matrix. Therefore, the biomolecules of interest are slowly removed from the cell-laden matrix and hence the cells). In contrast, prior art methods for analysis of biomolecules of interest predominantly do not comprise a matrix that surrounds the cell(s), so that biomolecules of interest are directly and quickly captured. This can impede a physiological cell response, e.g. intra-cellularly or inter-cellularly, e.g. between two cells to be analysed. Hence, the at least two matrices used according to the present invention allow to provide physiological environments (including a physiological cell signalling) in order to establish of dynamic cell-cell-interactions and autocrine signalling. The subsequent quantification of secreted biomolecules advantageously can prevent capture effects.

The Cell Culture Device

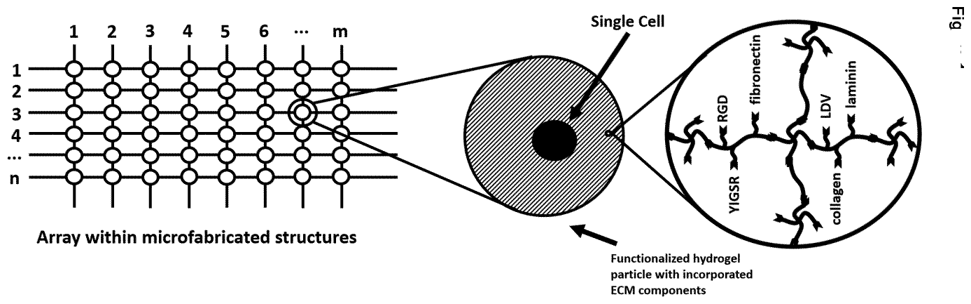

According to a preferred embodiment, the provision of a cell-laden matrix and a capture matrix is performed utilizing a cell culture device. Preferably, the cell culture device is a microfabricated cell culture device. According to one embodiment, the cell culture device comprises at least one compartment, preferably an array of compartments, wherein an array corresponds to a plurality of compartments that are ordered. Preferably, the order of the plurality of compartments comprises positions of x- and y-coordinates (e.g. n×m array). An exemplary plurality of compartments (also referred to as an array) is depicted in FIG. 1 and it is referred thereto. Other orders including no order (e.g. random order) of compartments are also within scope of the present disclosure.

The at least one compartment is typically connected to channels, through which transport can be performed. According to a preferred embodiment, the device comprises a fluid reservoir and fluid channels for providing fluid to the at least one compartment. For instance, fluid can be transported through the channels into the compartments and then further out of the compartments to either a subsequent compartment (e.g. following compartment of a plurality of compartments) or a waste or a reservoir. The fluid may also be transported to other positions of the cell culture device (e.g. a storage position, etc.). In addition to the fluid (which can be an aqueous fluid, such as cell culture media, or a non-aqueous fluid, such as fluorinated oil), other components can be transported through the channels (e.g. inside the fluid). According to a preferred embodiment, matrices, including cell-laden matrices and capture matrices can be transported through the channels. Moreover, cell suspensions and solutions which are capable of crosslinking or being crosslinked can be transported through the channels. Hence, the at least one (microfabricated) compartment can be perfused with different solutions in a controlled manner, allowing for instance, washing of the matrices and perfusion with cell media. For instance, washing may be beneficial in scope of the method of the first aspect in order to remove unbound molecules which would give false positive results if not removed. According to one embodiment, a plurality of compartments is provided in an array, wherein compartments can advantageously share common inlets (e.g. feeling line), allowing matrices (e.g. the capture matrices) being positioned using one feeding line. This advantageously increases the speed of the disclosed method.

According to one embodiment, at least one cell is first encapsulated by utilizing the cell culture device in one droplet, which forms the matrix after droplet generation. Also the capture matrix may be formed by generating a droplet utilizing the cell culture device, followed by matrix generation of the droplet. A formed cell-laden matrix and/or capture matrix may then be transported through the channels to a compartment, wherein the matrices can be positioned in proximity to each other (also referred to as accommodated). According to one embodiment, the device comprises at least one compartment for accommodating at least one, preferably at least two matrices, including at least one capture matrix and/or at least one cell-laden matrix. According to one embodiment, the device comprises a compartment for accommodating at least one matrix, preferably two matrices, wherein a microfabricated geometry for matrix immobilization is present suitable for positioning the at least one matrix. According to one embodiment, a plurality of compartments for accommodating at least one matrix, preferably by an array of compartments is comprised in the cell culture device.

According to a preferred embodiment, a plurality of cell-laden matrices and capture matrices are provided in a cell culture device comprising a plurality of compartments, wherein at least one cell-laden matrix and at least one capture matrix are provided within a compartment of the cell culture device. According to a preferred embodiment, the cell-laden matrix and the capture matrix are provided in proximity within a compartment of a device. Alternatively, the cell-laden matrix and the capture matrix are provided in separate compartments, wherein the separate compartments are in fluid communication with each other or can be brought in fluid communication with each other (e.g. by the operation of a valve) so that the released biomolecules of interest can contact the capture matrix for capturing. According to a preferred embodiment, the cell-laden matrix and capture matrix are located in proximity, preferably in close proximity, at a defined position (e.g. in one compartment, in particular in a microfabricated compartment at position (n|m) of an n×m array of microfabricated compartments). This is particularly advantageous, as the proximity between said matrices allows for diffusion of biomolecules of interest between the said matrices (e.g. released biomolecules of interest derived from single or multiple cells located within the cell-laden matrix can diffuse through the matrix to the neighboring capture matrix). Hence, it is possible to achieve high capture efficiency of released biomolecules of interest due to short diffusion distances between two matrices. Moreover, the theoretical reduction of reaction volume can increase the sensitivity by increasing concentrations (e.g. by increasing the local concentration and thus capturing efficiency). According to a particular embodiment, the reaction volume may be further reduced by replacing an aqueous phase that may surround the matrices (in particular the cell-laden matrix) with a water-immiscible phase (e.g. oil phase) to generate a matrix comprising a shell of said water-immiscible phase (e.g. alternating biphasic compartment generation). Thereby advantageously, the reaction volume can be reduced to increase locally the concentration of the biomolecules of interest as these are hindered in diffusing out of the matrix by said shell. Furthermore, a capture matrix may be provided in close proximity (e.g. in direct contact to the cell-laden matrix) to enable diffusion from the cell-laden matrix. Hence, a detection mechanism with higher sensitivity may be achieved. According to another embodiment, the cell-laden matrix and capture matrix can be separated from each other by distance and/or time (e.g. by providing the capture matrix and cell-laden matrix in different compartments). Accordingly, at least one cell-laden matrix and at least one capture matrix are positioned preferably by a microfabricated geometry for matrix immobilization inside a compartment, wherein the compartment accommodating the at least one cell-laden matrix is different from the compartment accommodating the at least one capture matrix and wherein both compartments can be switched to be either in fluid contact with each other or to be in no fluid contact with each other. In order to still allow capture of biomolecules of interest, the cell-laden matrix and capture matrix may be provided in neighboring compartments that can be selectively brought into fluid contact with each other (e.g. by a valve, preferably a microfabricated valve). Thereby, advantageously, cell cultivation under physiological conditions is separated from further reactions (e.g. from step c) and/or d)). Moreover, capture effects may be avoided ensuring physiological environments (for improved signaling).

In addition, the matrices can be selectively removed from the compartment (e.g. the capture matrix can be independently from the cell-laden matrix transported in and out of the compartment). According to one embodiment, the method comprises obtaining capture matrices from a plurality of compartments and transfer of the capture matrices to a device comprising a plurality of compartments. For instance, the capture matrix may be removed from a compartment, while the cell-laden matrix stays inside the compartment, whereupon the capture matrix can be transported to a storage position (also referred to as a compartment of a device which is not the compartment comprising the cell-laden matrix). Another capture matrix can then be added if desired. Moreover, a capture matrix within a compartment located within an array can be removed without removing capture matrices located within other compartments of an array of compartments. Thereby, capture matrices can be obtained in a controlled manner and the position information is advantageously preserved (e.g. by selectively obtaining capture matrices of a particular compartment and transferring the capture matrix into a separate well of a well plate). In case multiple capture matrices are obtained, each capture matrix is transferred to a storage position, wherein the storage positions of matrices from different compartments are preferably different. The storage position may be any position capable of storing a matrix, including a position on the cell culture device (it was initially provided to) or a position outside of the cell culture device (e.g. by transporting the matrix outside the cell culture device into another format, such as a well of a well plate, e.g. a collection well). Transferring the capture matrix to a storage position at which the capture matrix can be perfused independently from the cell-laden matrix has the advantage, that the capture matrix can be washed/processed without affecting the cell-laden matrix/matrices. Thus, the cell behaviour, respectively the cell-laden matrix is not affected by processing of the capture matrix.

According to one embodiment, after obtaining a capture matrix from a compartment comprising the cell-laden matrix, another capture matrix comprising one or more types of capture molecule (but preferably no biomolecules of interest bound thereto) may be provided and transferred to said compartment. Positioning and transfer of fluids and other components (e.g. the matrices) may be performed utilizing a cell culture device comprising compartments comprising a trapping geometry. According to a preferred embodiment, the device comprises a microfabricated geometry for matrix immobilization inside a compartment for releasably positioning at least one matrix.

According to one embodiment, the device comprises a microfabricated geometry for matrix immobilization inside a compartment, wherein the geometry for matrix immobilization has one or more of the following characteristics:
it is capable of positioning the cell-laden matrix and the capture matrix in proximity;
it is capable of positioning at least two cell-laden matrices and the capture matrix in proximity;
it is capable of positioning the cell-laden matrix and at least two capture matrices in proximity; or
it is capable of positioning at least two cell-laden matrices and at least two capture matrices in proximity.

The trapping geometry (which may also be referred to as a positioner or positioning means), enables immobilization of single or multiple matrices (including two, three, four or more matrices, preferably two or three matrices) in a pre-defined/controlled manner. Such a configuration advantageously enables positioning of a capture matrix and a cell-laden hydrogel matrix in a very controlled manner within the same compartment thereby allowing the capture of released biomolecules of interest. According to one embodiment, multiple cell-laden matrices and capture matrices are provided and positioned (preferably at least one of each matrix kind) by a trapping geometry inside a compartment, wherein multiple such compartments are provided (i.e. in an array). Such an embodiment advantageously allows to quantify simultaneously/in parallel released biomolecules of interest from pre-defined cell compositions provided by the cell-laden matrices in a highly multiplexed manner.

The trapping geometry may comprise a valve arrangement adapted to provide a fluid passing through a microfabricated geometry for matrix immobilization. In one embodiment, the device comprises a trapping geometry comprising a valve arrangement adapted to provide a fluid passing through a microfabricated geometry for matrix immobilization wherein the valve arrangement is adapted to selectively change the direction of fluid passing the microfabricated geometry for matrix immobilization, in particular wherein a fluid a first direction urging the at least one matrix into the microfabricated geometry for matrix immobilization and a fluid in the second direction urging the at least one matrix out of the microfabricated geometry for matrix immobilization, and in particular fluid in the second direction delivering the at least one matrix in direction of an exit section. Such a configuration can advantageously transfer one or more matrices inside the compartment. Such a configuration can further advantageously be utilized for obtaining one or more matrices from the compartment. The mechanism of such a configuration may rely or may also be referred to as a reverse flow cherry picking (RFCP) mechanism. Such a valve arrangement allows to transfer fluid which may comprise individual matrices from and into a compartment. Therefore, capture matrices can be transferred after a pre-defined period into another format, while the position information is maintained allowing to correlate the release profile of biomolecules of interest with the corresponding cell(s).

According to a preferred embodiment, the valve arrangements are based on microfabricated valves. These are disclosed below and also apply here. A microfabricated valve may be capable of switching the compartment to an open or closed state. According to one embodiment, a microfabricated valve comprises a first channel, a second channel, a connection channel connecting the first channel and the second channel, a valve portion arranged within the connection channel, wherein the valve portion is adapted to selectively open and close the connection channel. According to one embodiment, a microfabricated valve comprises at least three layers, wherein a first channel is located within a first layer; a second channel is located within a third layer; a valve portion is located within a second layer; the second layer is arranged between the first and the third layer. A device may comprise a microfabricated valve, wherein a first channel comprises a microfabricated geometry for matrix immobilization suitable for positioning at least one matrix being contained in a fluid which flows through the first channel, wherein the microfabricated geometry for matrix immobilization is arranged within the first channel in such a way that a fluid flow can be reduced by the microfabricated geometry for matrix immobilization, in particular, the microfabricated geometry for matrix immobilization narrows the cross section of the channel; and/or wherein a second channel comprises a microfabricated geometry for matrix immobilization suitable for positioning particles being contained in a fluid which flows through the second channel, wherein the microfabricated geometry for matrix immobilization is arranged within the second channel in such a way that a fluid flow can be reduced by the microfabricated geometry for matrix immobilization, in particular, the microfabricated geometry for matrix immobilization narrows the cross section of the channel.

In order to continuously provide nutrition to the cell-laden matrix (and thus to the at least one cell), the compartments, wherein the cell-laden matrix is accommodated, may be switched to an open state to provide fluid to the compartment. Alternatively, the compartment may be switched to a closed state (also referred to as an isolated state) to provide an isolated compartment, which has the advantage to not flush away secreted biomolecules of interest. Moreover, an isolated compartment can hold both the cell-laden matrix and the capture matrix inside the compartment (e.g. in a defined volume, i.e. closed reaction volume), wherein released biomolecules of interest are not lost, due to removal or exchange of fluid. Therefore, preferably isolated compartments are provided throughout step b), preventing loss of one or more biomolecules of interest. Yet, the method may not be limited to isolated compartments, as also compartments in the open state may be applicable, e.g. in case the flow rate is kept low enough or no flow is provided, allowing biomolecules of interest to remain in proximity to the cell-laden matrix and/or the capture matrix. According to a preferred embodiment, released biomolecules of interest diffuse within the isolated compartment and can be advantageously captured within the compartment for further analysis/processing. According to one embodiment, the device comprises at least one compartment that is capable of being switched between an isolated and an open state, wherein the isolated state corresponds to a state at which fluid that is present in the compartment is in no contact with fluid not present in the compartment and wherein the open state corresponds to a state at which fluid that is present in the compartment is in contact with fluid not present in the compartment.

The cell-laden matrix is preferably incubated to allow release of the one or more biomolecules of interest. Incubation may occur over a defined time period. Preferably, the incubation is performed by utilizing a cell culture device, which is preferably a microfabricated cell culture device. The cell-laden matrix, in particular, the at least one cell can be incubated inside a compartment of the cell culture device. Therefore, suitable conditions for incubating/cultivating are preferably applied, including but not limited to supply of one or more of a suitable temperature (e.g. 37° C. for human cells), CO₂-level (e.g. around 5% for human cells) and humidity. As disclosed herein, such incubation may also occur before the capture matrix is added for binding the released biomolecule(s) of interest.

According to one embodiment, the provided cell-laden matrix and capture matrix are provided with a fluid, preferably a fluid that is immiscible with water, wherein said matrices, provided with said fluid, are preferably generated by utilizing a cell culture device, which preferably is a microfabricated cell culture device, and preferably by
(i) releasably positioning the cell-laden matrix and the capture matrix by a preferably microfabricated geometry for matrix immobilization inside a compartment, wherein the compartment comprises a first fluid, preferably an aqueous fluid;
(ii) removing the first fluid from the compartment and replacing the first fluid by a second fluid that provides said fluid, wherein said fluid is preferably immiscible with water; and
(iii) optionally, removing the second fluid from the compartment and replacing it by the first fluid or a third fluid, that is preferably immiscible with the second fluid.

According to one embodiment, the cell-laden matrix and the capture matrix may be confined in a volume that is smaller than the volume of a compartment, in which the matrices may be advantageously positioned (e.g. by a microfabricated geometry for matrix immobilization). Such a reduction in volume can preferably be achieved by providing the cell-laden matrix and the capture matrix in a fluid (which may be referred to as second fluid or the “said fluid” when referring to claims 23 and 24), which is immiscible with the fluid comprised in the matrix (referred to as first fluid, e.g. an aqueous fluid). Thereby, the available volume accessible for the released biomolecules of interest for diffusion is reduced to the volume of the matrices (respectively the first fluid comprised in the matrices) and optionally, the volume of the first fluid that still surrounds the matrix (e.g. in form of a shell, e.g. water-shell or aqueous shell). This advantageously allows to avoid dilution effects and the local concentration of the released biomolecules of interest is increased. The second fluid can advantageously reduce the available volume for diffusion of the biomolecules of interest and thus circumvent the resolution limit a microfabricated cell culture device may have (e.g. the resolution of fabrication). The cell-laden matrix and the capture matrix may preferably be positioned inside a compartment of the cell culture device, wherein the matrices are preferably in close proximity, more preferably in direct contact. The compartment may initially comprise a first fluid (e.g. a cell culture medium). Afterwards, the first fluid may be removed and replaced by a second fluid, which is immiscible with the first fluid (e.g. a fluorinated oil). In the next step, optionally, the second fluid may be removed and replaced by the first fluid or by a third fluid, wherein the third fluid is preferably immiscible with the second fluid. According to one embodiment, the first and/or third fluid may be selected from an aqueous fluid, including aqueous solutions capable of being contacted with the cell-laden matrix, preferably without causing severe apoptosis of the cells (unless desired). In such an embodiment, the cell-laden matrix and the capture matrix are preferably present in a volume of second fluid, which is shared by the matrices and the cell-laden matrix and capture matrix are preferably in direct contact with each other to enable direct diffusion of biomolecules of interest.

According to one embodiment, the cell-laden matrix is incubated to allow release (e.g. secretion) of one or more biomolecules of interest before providing the capture matrix in step (a). After providing the capture matrix, one or more biomolecules of interest are specifically bound by the one or more types of capture molecules of the capture matrix. The cell-laden matrix is preferably provided in a defined volume of a fluid, preferably a fluid that is immiscible with water, and wherein the capture matrix is provided in a defined volume of the same type of fluid, and wherein after contacting the cell-laden matrix and the capture matrix said fluids of the same type merge to provide a defined volume of fluid that is shared by the cell-laden matrix and the capture matrix. As disclosed herein, the capture matrix may be positioned in contact with the cell-laden matrix before, during or after such incubation.

According to one embodiment, the cell-laden matrix is incubated before providing the capture matrix in step (a). Such an incubation step can be performed by utilizing a cell culture device, preferably a microfabricated cell culture device. The incubation can take place as described herein. According to a preferred embodiment, the cell-laden matrix is provided in a defined volume of a fluid (also referred to as second fluid), wherein said fluid is immiscible with a first fluid, wherein the first fluid is the fluid present in the cell-laden matrix, which is preferably an aqueous fluid (e.g. cell culture medium). Preferably, the second fluid is immiscible with water. Thereby, the available volume accessible for the released biomolecules of interest for diffusion is reduced to the volume of the matrices (respectively the first fluid comprised in the matrices) and optionally, the volume of the first fluid that still surrounds the matrix (e.g. in form of a shell, e.g. water-shell or aqueous shell). According to one embodiment, the capture matrix is provided in step a) after incubating the provided cell-laden matrix in a defined volume of a fluid (e.g. second fluid). The incubation may be performed by utilizing a cell culture device comprising a microfabricated geometry for matrix immobilization, wherein preferably the cell-laden matrix is immobilized and is provided inside the second fluid. After incubating the cell-laden matrix, a capture matrix may be preferably provided in a defined volume of the same type of fluid. In this case, the same type of fluid corresponds to the type of fluid the cell-laden matrix was provided in/with. The defined volumes of the fluid (e.g. second fluid) that the matrices are provided in/with may be any volume of fluid as long as the volume of fluid is capable of at least partially surrounding the matrices. In a preferred embodiment, the volume of fluid is capable of fully surrounding the matrices. After providing the capture matrix provided in a defined volume of the same type of fluid, said capture matrix, respectively capture matrix provided in a defined volume of the same type of fluid is contacted with the cell-laden matrix, provided in a defined volume of the fluid (e.g. second fluid). When the matrices contact each other, respectively the provided fluids of the same type (e.g. second fluids), the fluids merge (which may also be referred to as coalesce) to provide a defined volume of fluid that is shared by the cell-laden matrix and the capture matrix. The shared volume may be any volume as long as it is at least partially, preferably fully, capable of surrounding said matrices. It may correspond to the sum of the defined volumes provided or may be less than the sum or more than the sum. The capture matrix may be provided such that the cell-laden matrix and the capture matrix are in close proximity. Preferably, the cell-laden matrix and the capture matrix are in direct contact with each other, advantageously allowing for a direct diffusion of biomolecules of interest from the cell-laden matrix to the capture matrix. A direct contact between the cell-laden matrix and the capture matrix may be established by the microfabricated geometry for matrix immobilization inside a compartment. For instance, a cell-laden matrix may be positioned directly next to a capture matrix by the microfabricated geometry for matrix immobilization inside a compartment. The delayed provision of the capture matrix has the advantage that released biomolecules of interest can first accumulate within the cell-laden matrix provided in a defined volume of the second fluid (e.g. biomolecules accumulate in the aqueous fluid present in the cell-laden matrix and optionally a surrounding aqueous shell, which may also be referred to as the first fluid), as the biomolecules of interest may be less soluble in the second fluid, which is immiscible with the first fluid (e.g. immiscible with water). In a particular embodiment, more than one cell-laden matrix is provided in a defined volume of a fluid (e.g. second fluid). These may be preferably in close proximity, more preferably in direct contact with each other to allow for a direct diffusion of biomolecules of interest between the cell-laden matrices. For instance two cell-laden matrices or three cell-laden matrices may be provided, wherein the at least one cell of each cell-laden matrix can be of the same type or different. In a particular example, the cells may be different in order to study their interaction. The provision of the cell-laden matrices provided in a defined volume of a fluid (e.g. second fluid) allows for a paracrine and autocrine signaling of cells and avoiding capture effects (which can occur when biomolecules of interest are directly captured; see disclosure above for further details about the capture effect). Only when the capture matrix is provided and positioned in close proximity, preferably in direct contact, with one or more cell-laden matrices, the released biomolecules of interest bind to the one or more type of capture molecules.

In one embodiment the cell culture device is a conventional cell culture flask or cell culture plate such as a 12-well plate, a 24-well plate, 96-well plate, a 384-well plate.

According to one embodiment, the cell-laden matrix is provided in a cell culture device selected from a cell culture flask or cell culture plate, such as a 12-well plate, a 24-well plate, 96-well plate, a 384-well plate. In one embodiment, the cell-laden matrix is provided in a compartment of the cell culture plate, e.g. a well. Per compartment, one or more cell-laden matrices (e.g. 1, 2, 3 or more, optionally 1) may be provided. The cell-laden matrix can be positioned in the compartment of the cell culture plate such that liquid which may surround the cell-laden matrix can be exchanged without affecting the cell-laden matrix, e.g. without contacting or disrupting the cell-laden matrix. For instance, the cell-laden matrix can be provided inside the compartment of the cell culture plate leaving an outer rim allowing liquid to be accessed, e.g. by a pipette, without affecting the cell-laden matrix. The cell-laden matrix is optionally incubated before being in fluidic contact with the capture matrix. For instance, the capture matrix may placed in a separate compartment or containment (e.g. tube). After incubating the cell-laden matrix to release the biomolecule(s) of interest, the capture matrix may be added for binding the released biomolecule(s) of interest. First incubating the cell-laden matrix prior to contacting the released biomolecule(s) of interest with the capture matrix allows that the released biomolecule(s) of interest may first accumulate in the compartment of the cell culture plate before binding to the one or more types of capture molecules of the capture matrix. However, different contacting orders of capture matrix and cell-laden matrix are possible and within the scope of the present disclosure, as disclosed herein.

According to one embodiment, the cell-laden matrix is analyzed by optical analysis throughout the incubation, wherein methods and devices for optical analysis are well-known in the art. An exemplary optical analysis may be microscopic analysis. In a preferred embodiment, optical analysis can be advantageously performed in combination with the cell culture device (e.g. the cell-laden matrix can be optically analyzed by microscopy when present in the cell culture device, preferably present in a compartment of a cell culture device).

Further details of the here described subject-matter, including the cell culture device, channels, microfabricated valve, and valve arrangement are disclosed further below in the further embodiments of the method of the first aspect and it is here referred to the respective disclosure which also applies here.

Step c)

According to step c) of the method of the first aspect, one or more types of detection molecules are added, wherein each type of detection molecule specifically binds a biomolecule of interest, and wherein each type of detection molecule comprises a barcode label which comprises a barcode sequence (B_{S}) indicating the specificity of the detection molecule.

The one or more types of detection molecules according to the present disclosure specifically bind to biomolecules of interest. The one or more types of detection molecules may preferably bind to the biomolecules of interest that were previously captured by the capture matrix (see also FIG. 2). In one embodiment, a type of detection molecule binds its biomolecule of interest at a different region (e.g. site of recognition, chemical moiety, etc.) than the corresponding type of capture molecule that binds the same biomolecule of interest. For instance, a first type of capture molecules binds to particular biomolecules of interest (e.g. antigen molecules X) in step a) and b). Then, in step c) a first type of detection molecule binds to the particular biomolecules of interest, but to a different region of the biomolecules of interest (e.g. a different epitope of antigen X). In this respect it is further referred to FIG. 2A, which schematically illustrates such binding (in particular FIG. 2A, step C). Examples and preferred embodiments for detection molecules applicable in step c) are further described in detail below and also apply here.

According to a preferred embodiment, one type of detection molecules comprises multiple detection molecules of the same type. In one embodiment, the one or more types of detection molecules do not directly bind to the one or more types of capture molecules, as in such case, also capture molecules would be bound by detection molecules that have not bound to a biomolecule of interest.

The addition of the one or more types of detection molecules is in one embodiment performed by utilizing the cultivation device, which is preferably a microfabricated cultivation device. The one or more types of detection molecules may be introduced into the cultivation device and in particular into the compartments accommodating the one or more cell-laden matrices and capture matrices. This has the advantage to directly provide the one or more types of detection molecules to the capture matrices without requiring transferring the capture matrix before addition of the detection molecules (which is however, an option). Moreover, by directly introducing the one or more types of detection molecules, only small volumes of liquid may be required, as the compartments and channels of the cell cultivation device are relatively small in comparison to standard cell culture dishes. Hence, material is saved. Introducing detection molecules in such a manner has the further advantage of enabling multiplexing, wherein two or more types of detection molecules can be introduced in a multiplexed manner—without the necessity to perform further liquid handling processes. However, it may also be within the scope of the present disclosure, to first transfer the capture matrix to a storage position, whereupon the one or more types of detection molecules are added. In such an embodiment, the direct contact between the one or more type of detection molecules and the cell-laden matrix can be avoided.

According to a one embodiment, the compartment comprising one or more matrices (preferably, the at least one cell-laden matrix and the at least one capture matrix) is washed by perfusion with a washing solution to remove unbound compounds. Compartments may also be washed which do not comprise a matrix (e.g. in form of a pre-perfusion). According to a particular embodiment, the capture matrix in proximity to the cell-laden matrix is washed after a pre-defined incubation time with a solution comprising one or more types of detection molecules. As a result the binding equilibrium of biomolecules of interest is simultaneously achieved to washing and no additional incubation time is required (in comparison to prior art methods, such as an external assay), which can be more time-efficient.

The number of types of detection molecules may be the same as the number of types of capture molecules or may be different. Preferably, the detection molecules are used in excess to allow efficient and quantitative binding of the captured biomolecules of interest.

The Barcode Label

A detection molecule comprises a barcode label which comprises a barcode sequence (B_{S}) indicating the specificity of the detection molecule. The barcode label is advantageously attached to the detection molecule. Particular embodiments of attachment of the barcode label to the detection molecule are described in detail below in conjunction with the further embodiments of the method of the first aspect. The barcode label preferably comprises an oligonucleotide. In one embodiment, the barcode label attached to the one or more types of detection molecules comprises DNA, RNA, PNA, LNA or combinations thereof. Preferably, the barcode label comprises a DNA sequence. The attachment between the detection molecule and the barcode label may be covalent and preferably is cleavable (e.g. photocleavable). Such an embodiment has the advantage, that the barcode label can be easily separated from the capture molecule and thus quickly accessible for further analysis or processing. Thus, the barcode label may be attached to the detection molecule via a linker moiety, such as a cleavable linker. Such an embodiment has the advantage that it can be directly amplified in subsequent amplification processes (e.g. PCR reaction). For instance, a primer or primer combination may be provided in order to hybridize to the barcode label, and thereby generate a template for a polymerase reaction.

The detection molecule is labelled with a barcode label B_{S} encoding the specificity. This enables high analysis sensitivity, as a barcode label comprising a barcode sequence B_{S} can be detected in further analysis with great sensitivity. Moreover, the use of a barcode sequence B_{S} allows to analyze multiple biomolecules of interest in parallel (multiplexing), as the barcode sequence B_{S} advantageously is specific for each type of detection molecule and thus is specific for the targeted biomolecule of interest. Thus, the barcode sequence B_{S} allows to identify later (e.g. based on sequencing) which biomolecule of interest was released by the cell and captured by the capture matrix and subsequently bound by a detection molecule.

Different types of detection molecules are labelled with different barcode sequences B_{S}. Advantageously, this allows to differentiate between different types of detection molecules (and thus released biomolecules of interest) on the basis of the barcode sequence B_{S}.

According to one embodiment, the method according to the first aspect comprises analyzing at least two (e.g. 2 to 100, 5 to 50, 5 to 25, 5 to 20 or 7 to 15) different biomolecules of interest using different types of capture molecules and different types of detection molecules, wherein the barcode label of each type of detection molecule that binds a biomolecule of interest differs in its barcode sequence B_{S} from the barcode sequence B_{S} of all other types of detection molecules that bind a different biomolecule of interest. This advantageously allows to generate in the subsequent step d) described below different sequenceable reaction products that comprise different barcode sequences B_{S}, thereby allowing to specifically identify the bound biomolecule of interest. According to one embodiment, the barcode label of the detection molecule and thus also the barcode sequence B_{S} is advantageously compatible with commercial NGS assays and can be easily processed using common NGS platforms. Therefore, the information encoded in the barcode label comprising the barcode sequence B_{S} can be quickly read out by sequencing the generated reaction product to identify which barcode sequence B_{S} is present and thus which detection molecule and respective bound biomolecules of interest are present. On the basis of such information (i.e. the barcode sequence B_{S}), the barcode label allows to identify which biomolecules of interest were bound to the capture matrix and were therefore released by the at least one cell during the incubation period. Moreover, the use of two or more types of detection molecules comprising a barcode label with a unique barcode sequence B_{S} to indicate the target specificity enables multiplexing, wherein multiple biomolecules of interest can be detected with one capture matrix that contains two or more types of capture molecules (e.g. screening for multiple cytokines such as TNF-α, IL-6, IL-10, Il-1) or at least two capture matrices, wherein each capture matrix comprises a single type of capture molecule. The collected capture matrix can also be used for quantifying the bound biomolecule of interest by detecting the barcoded label (e.g. oligonucleotide) associated to the detection molecule (e.g. antibody). This can for instance, be done by qPCR or digital PCR. Another possibility might be to amplify such oligonucleotides and then sequence an amplified product (e.g. with nanopore sequencing or similar techniques). A robust method for quantification involves the use of an UMI barcode sequence as described elsewhere herein.

The barcode label comprising the barcode sequence B_{S} is added and preferably attached to the detection molecule. In case two or more types of detection molecules are used, the barcode label is attached to its detection molecule at least before mixing more than one type of detection molecules. The barcode sequence can be added during or after detection molecule production (e.g. commercially available antibodies). The barcode labelling can be easily performed by methods available in the art and described below in the further embodiments of the method of to the first aspect. Labelling can be achieved with a high yield for multiplexing. Preferably, each detection molecule is labelled with only one barcode label, comprising the barcode sequence B_{S}. Labelling with exactly one molecule can be advantageous if an absolute quantification of biomolecules of interest is desired (which typically is achieved in conjunction with the use of an UMI sequence as described further below but can also be achieved by labelling with exactly one barcode label comprising a barcode sequence B_{S} and further processing, e.g. by q-PCR). However, it may also be within the scope of the present disclosure to label detection molecules with less than one barcode label or multiple barcode labels per detection molecule. For instance, the detection molecule may be labelled with two or more barcode labels in order to enhance the detectable signal (e.g. have more sequenceable material for subsequent sequencing).

After performing step c), the amount of detection molecules comprising a barcode label bound to/associated with the capture matrix may be and is preferably proportional to the amount of biomolecules of interest bound by the capture molecules attached to the capture matrix. Advantageously, the detection molecules comprising the barcode label remain associated with the capture matrix by binding to the capture molecule-bound biomolecules of interest. This advantageously allows to indirectly quantify the biomolecules of interest (e.g. via the bound detection molecules).

According to one embodiment, the barcode label attached to a detection molecule comprises a barcode sequence (B_{S}) indicating the specificity of the detection molecule. In addition, the barcode label may comprise one or more of the following sequence elements:

One or more primer target sequences (e.g. primer sequence (1) or (2) or primer sequence (1) and primer sequence (2)). Primer sequences may be advantageously incorporated into the barcode label in order to simplify or enable amplification in a subsequent amplification reaction (e.g. PCR reaction). Suitable embodiments are illustrated in the figures.

A barcode sequence (B_{T}) indicating a time information. The barcode sequence B_{T} may be advantageously applied in order to indicate a particular time point at which the biomolecules of interest are captured (e.g. after an incubation period tₓ₁). Different barcode sequence B_{T} are used for different time points or incubation periods. This allows to analyze the released biomolecules of interest over different time points or periods. For instance, a B_{T} sequence may be applied that comprises identifiable information (identifiable by sequencing). The barcode label comprising the barcode sequence B_{T} is thus specific for a time point of analysis (e.g. incubation time tₓ₁). By applying different B_{T} sequences for different time points/incubation periods, the barcode labels of multiple time points can be advantageously collected together (e.g. in one collection position) for further combined processing, as the barcode sequence B_{T} allows to differentiate the barcode labels according to the time points/incubation periods.

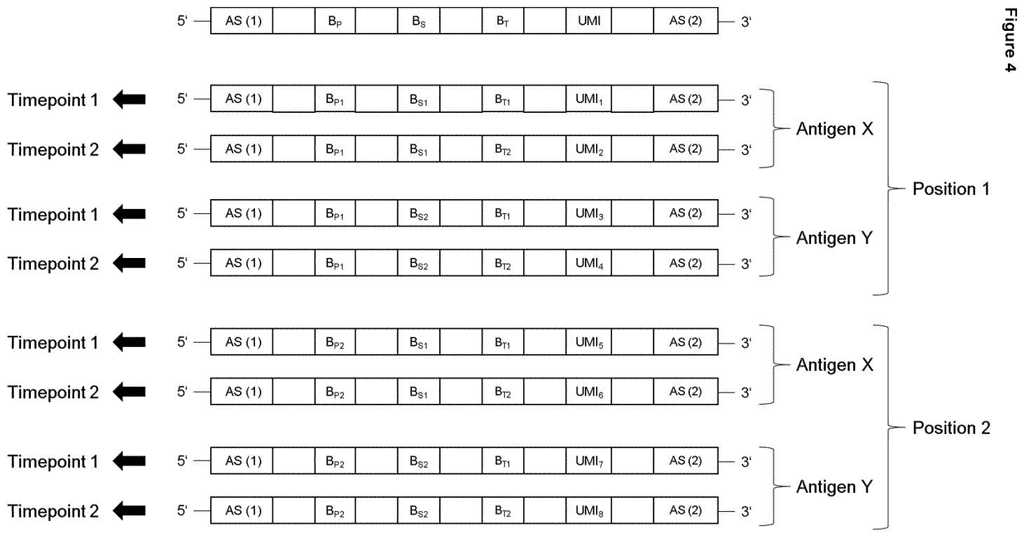

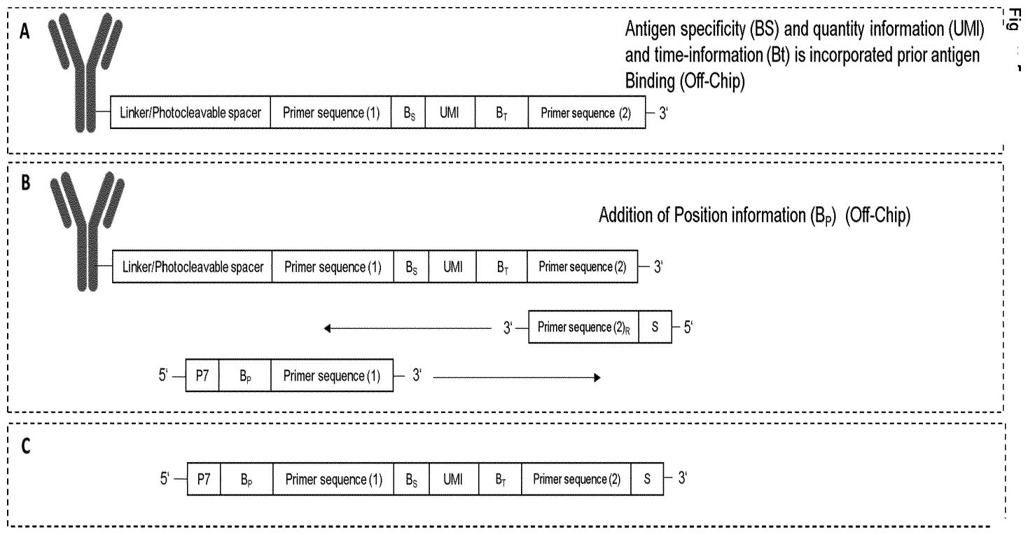

A unique molecular identifier (UMI) sequence (see e.g. FIGS. 5 and 6). Each detection molecule, respectively barcode label, within one “reaction composition” preferably comprises a unique UMI sequence, thereby allowing to identify each bound detection molecule based on the UMI sequence. Conjugated detection molecules having a barcode sequence for their specificity are commercially available (e.g. from Biogen) and can be easily modified to include UMI sequences. Degenerate synthesis of oligonucleotides might be used for UMI synthesis, as well as semi-random sequencing approaches. E.g., they may be designed as a string of totally random nucleotides (such as NNNNNNN), partially degenerate nucleotides (such as NNNRNYN), or defined nucleotides. According to one embodiment, the UMI is provided by a semi-random sequence consisting of (Xmer)n, wherein n is an integer from 2-8. Such approaches are well-known from next generation sequencing applications, wherein UMI barcode sequences are widely used for identifying a single template molecule and quantification. The direct incorporation of the UMI into the barcode label of the detection molecule eliminates the need for an adapter barcode oligonucleotide to add further information in form of a UMI sequence by hybridization and subsequent polymerase extension reaction to incorporate the UMI sequence into the barcode label. Thus, the direct incorporation of the UMI into the barcode label reduces the processing time. Each comprised UMI sequence is preferably different for each detection molecule, thereby allowing to specifically identify the bound detection molecule and thus, the biomolecule of interest. Different detection molecules (on the molecule level) comprise different UMI sequences. If one or more further sequence elements are provided for differentiation (as is disclosed herein), it is not necessary that the UMI sequence is different for every molecule, as differentiation can be achieved through the further sequence element(s) (e.g. B_{T}). However, for a plurality of barcode label molecules with an equal set of sequence elements, the UMI sequence is preferably different for each molecule. In respect to the present disclosure, an UMI sequence may be advantageously applied to directly quantify the absolute number of biomolecules of interest bound by one or more types of detection molecules. Therefore, an UMI sequence can be provided in the barcode label, allowing to quantify the number of detection molecules bound to the biomolecules of interest and thus to quantify the number of biomolecules of interest. An UMI sequence may be present on the barcode label next to the barcode sequence B_{S}, and may be e.g. present 3′ of the barcode sequence B_{S}. However, the exact order of the barcode label may not be limited, e.g. the UMI sequence may also be 5′ of the barcode sequence B_{S}.

According to one embodiment which is also illustrated schematically in FIG. 6, the barcode label comprises the barcode sequence B_{S} and two primer sequences (e.g. primer sequence (1) at the 5′ end and primer sequence (2) at the 3′ end), as well as a unique molecular identified (UMI) sequence, which is located 3′ of the barcode sequence B_{S}.

According to one embodiment which is also schematically illustrated in FIG. 5, the barcode label comprises the barcode sequence B_{S} and two primer sequences (e.g. primer sequence (1) at the 5′ end and primer sequence (2) at the 3′ end), as well as a unique molecular identifier (UMI) sequence, which is 3′ of the barcode sequence B_{S}. The embodiment is not limited to the exact arrangement of sequence elements and other orders are within the scope of the present disclosure (e.g. the UMI sequence may be 5′ of the barcode sequence B_{S} or 3′ of the barcode sequence B_{T} or the barcode sequence B_{T} may be 5′ of the barcode sequence B_{S}, etc.). Such an embodiment combines the advantages described above. For instance, such a barcode has the advantage that it can be directly amplified in subsequent amplification processes (e.g. PCR reaction). For instance, a primer or a primer combination may be provided in order to hybridize to the barcode label (e.g. at the one or more primer sequences), and thereby generate a template for a polymerase reaction. Moreover, the presence of the UMI sequence enables quantification of the biomolecules of interest. In combination with the barcode sequence B_{T} the number of required UMI sequences can furthermore be reduced, as different B_{T} sequences (indicating the different time information) do not necessarily require different UMI sequences (e.g. UMI sequence 1 together with B_{T} can still be differentiated from UMI sequence 1 together with B_{T2}). Hence, the same UMI sequence may be applied for different barcode sequences B_{T}.

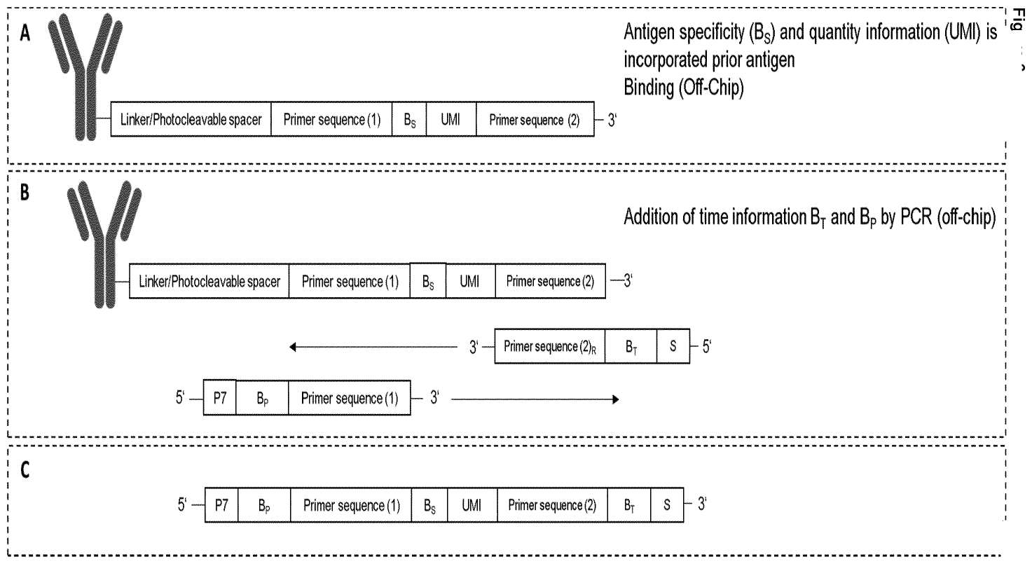

According to one embodiment, the barcode label comprises an adapter sequence, which can advantageously hybridize to an oligonucleotide, such as an adaptor barcode oligonucleotide. An adapter sequence has the advantage that it is capable of hybridizing to a desirable sequence, such as an oligonucleotide, in order to add further information in form of a sequence to the barcode label. An adapter sequence may preferably be at the 3′ end of the barcode label. According to one embodiment which is also schematically illustrated in the figures, the barcode label may optionally comprise the barcode sequence B_{S} and one primer sequence (e.g. primer sequence (1) at the 5′), as well as an adapter sequence (here adapter sequence (1)), which is 3′ of the barcode sequence B_{S}. Such an embodiment has the advantage that it is capable of hybridizing via the adapter sequence (1) to an oligonucleotide, which may contain information that can be transferred to the barcode label via a hybridization reaction (and an optional subsequence polymerase extension reaction). According to one embodiment which is also schematically illustrated in FIG. 7, the barcode label comprises the barcode sequence B_{S} and optionally at least one primer sequence (e.g. primer sequence (1) at the 5′), as well as an adapter sequence (here adapter sequence (1)), which may be locate at the 3′ end. Furthermore, an UMI sequence may be present 3′ or 5′ of the barcode sequence B_{S}. The exact order of the barcode labels is again not limited. Such an embodiment combines advantages described above, i.e. allowing for direct quantification of detection molecules (and thus bound biomolecules of interest) via the UMI sequence, while also other information may be added to the barcode label via hybridization of the adapter sequence.

According to one embodiment, the barcode labels of the one or more types of detection molecules all comprise the same adapter sequence. This allows to use a single type of adaptor barcode oligonucleotide for several or preferably all types of detection molecules. According to one embodiment, the barcode labels of the one or more types of detection compounds furthermore comprise the same one or more primer sequences. According to one embodiment, the barcode labels of the one or more types of detection molecules are all the same, except for the barcode sequence B_{S} that indicates the specificity of the detection molecules of the different types.

According to one embodiment, the barcode label comprises an adapter sequence AS for sequencing. Such adapter sequence AS may correspond to commercially available sequencing adapters, as they are regularly used in common sequencing platforms such as Illumina® sequencing. The sequencing adapter may be located in the 5′ region of the barcode label as is disclosed in conjunction with the figures. This enables e.g. a linear amplification in a subsequent amplification reaction wherein a single primer is used that comprises a matching sequencing adapter, while still providing a sequenceable reaction product comprising sequences adapter sequences at both ends, as are often required for common commercial sequencing platforms.

Step d)

In step d) of the present method, a sequenceable reaction product is generated, which comprises at least
(i) the barcode sequence (B_{S}), and
(ii) a barcode sequence (B_{T}) for indicating a time information, and/or
(iii) a barcode sequence (B_{P}) for indicating a position information, and
(iv) optionally a unique molecular identifier (UMI) sequence.

As is disclosed herein, step d) may comprise several substeps. Generation of the sequenceable reaction product may comprise the use of at least one oligonucleotide, optionally a primer, that is capable of hybridizing to the barcode label of the at least one type of detection molecule. This embodiment is preferred, and numerous suitable embodiments are disclosed herein. In a less preferred alternative, the generation of the sequenceable product in step d) comprises the use of at least one oligonucleotide that is ligated to the barcode label of the at least one type of detection molecule, whereby the barcode label is extended. This is an option to introduce further sequence elements to the thereby extended barcode label, such as e.g. barcode sequence (B_{T}) and/or a barcode sequence (B_{P}) and/or a UMI sequence. This allows for compatibility e.g. with Illumina® TruSight sequencing platform without interrupting the standard workflow. During library preparation, the adapters from the original library prep kit might be replaced by the disclosed adapters.

As already described, the barcode label comprises a barcode sequence B_{S}. The barcode label may be used as template to generate the sequenceable reaction product. If not already present in the barcode label, a barcode sequence (B_{T}) for indicating a time information and/or a barcode sequence (B_{P}) for indicating a position information must be added so that it is comprised in the sequenceable reaction product. As disclosed herein, the generated sequenceable reaction product preferably additionally comprises a unique molecular identified (UMI).

The generation of the sequenceable reaction product preferably comprises the use of at least one oligonucleotide that is capable of hybridizing to the barcode label of the at least one type of detection molecule. This oligonucleotide may be e.g. an adaptor barcode oligonucleotide or may be a primer that is used in an amplification reaction to generate and/or amplify the sequenceable product. Numerous suitable embodiments are described herein.

According to one embodiment, step d) comprises performing an amplification reaction using a primer or primer combination. Suitable embodiments are described herein and in the figures.

Use of an Adaptor Barcode Oligonucleotide

Step d) may additionally comprise extending the barcode label of the detection molecule using an adaptor barcode oligonucleotide capable of hybridizing to the barcode label as template, whereby an extended barcode label is provided in advance of the amplification reaction. Depending on the applied barcode label, the generation of the sequenceable reaction product may require the use of such adapter barcode oligonucleotide. Extension of the barcode label may be followed by a polymerase extension reaction, whereupon the information of the adapter barcode oligonucleotide is transferred to/incorporated into the extended barcode label when the barcode label is extended e.g. by a polymerase using the adapter barcode oligonucleotide as template. Such a barcode label hybridization and extension step may be here referred to as the sub-step (aa) of step d) (see also the schematic of such a step in FIG. 2A, step D (aa)).

According to a preferred embodiment, the adapter barcode oligonucleotide which may comprise for instance the barcode sequence B_{T}, the barcode sequence B_{P} and/or an UMI sequence, is preferably added to the capture matrix (comprising the captured biomolecules of interest bound by the detection molecules) after a washing step in order to remove unbound analytes (e.g. other non-bound biomolecules, unbound detection molecules, etc.). This advantageously allows to avoid false positive errors, for instance in view of an absolute quantification of the biomolecules of interest. The barcode labels may in embodiments also be released from the detection molecules prior to amplification and/or barcode label extension.

According to one embodiment, step (aa) takes place at one particular position, i.e. the hybridization and extension may take place at the same position, or at different positions, i.e. the hybridization may take place at one position and the extension may take place at another position. For instance, the hybridization of the oligonucleotide to the barcode label may take place in the compartment comprising the capture matrix, optionally in the presence of the cell-laden matrix, followed by an optional washing of unbound oligonucleotide, followed by a polymerase extension reaction by introducing the required reaction components into the compartment (e.g. polymerase, dNTPs, etc.). The hybridization of the oligonucleotide may be performed in the presence or absence of the cell-laden matrix, e.g. by transferring the capture matrix out of the compartment to another position prior to or after contacting the barcode label with the adapter barcode oligonucleotide (e.g. collection position of the cell culture device of another format such as a well plate). At the other position, the polymerase extension reaction may be performed in the presence of the required components (e.g. polymerase, dNTPs, etc.). Such an embodiment has the advantage that the cells are not influenced by the polymerase reaction and the required components. In one embodiment, the hybridization of the oligonucleotide may take place in absence of the cell-laden matrix by transporting the capture matrix to another position (e.g. see above) and then combine the capture matrix with the oligonucleotide, which is followed by the polymerase extension reaction.

The adapter barcode oligonucleotide may comprise an adapter sequence that is reverse complementary to the adapter sequence provided by the barcode label. Such an adapter sequence may be referred to as adapter sequence (1)_{R}, indicating that the sequence is reverse complementary to adapter sequence (1) of the barcode label. Preferably, the adapter sequence is located at the 3′ end of the adapter barcode oligonucleotide in order to hybridize to the barcode label 3′ end adapter sequence. However, other configurations may also be applied in scope of the present disclosure.

The adapter barcode oligonucleotide may comprise at least one primer sequence (e.g. primer sequence (2)) in order to incorporate into the extended barcode label a primer sequence that can be used in a subsequent amplification reaction. For instance, the adapter barcode oligonucleotide may comprise a primer sequence (2)_{R} indicating that the primer sequence is reverse complementary and thus would incorporate a primer sequence (2) into the barcode label after the polymerase extension reaction to which a primer can bind during the amplification reaction (see also discussion of the figures). According to one embodiment, an adapter barcode oligonucleotide comprising the adapter sequence (1)_{R}, preferably at the 3′ end and the primer sequence (2)_{R}, preferably at the 5′ end, is capable of hybridizing to a barcode label comprising an adapter sequence (1) at the 3′ end. Subsequently, a polymerase extension reaction may be performed in order to extend the barcode label and incorporate the information provided by the adapter barcode oligonucleotide into the extended barcode label. The extended barcode label then advantageously comprises the complementary sequences provided by the adapter barcode oligonucleotide overhang (e.g. a primer sequence (2), a barcode sequence B_{T}, a barcode sequence B_{P}, and/or an UMI sequence, etc. Particular embodiments of configurations of adapter barcode oligonucleotides are described further below.

The adapter barcode oligonucleotide may comprise further chemical moieties. For instance, it may comprise a blocking moiety. According to one embodiment, the adapter barcode oligonucleotide comprises a blocking moiety at the 3′ end, which blocks extension of the adaptor barcode oligonucleotide during extension of the barcode label. E.g. it may block a polymerase enzyme from polymerizing a complementary strand to which the adapter barcode oligonucleotide may have hybridized (e.g. the barcode label). Therefore, advantageously, the polymerization of a hybridized oligonucleotide (e.g. the barcode label) can be prevented at the 3′ end of the adapter barcode oligonucleotide. The 3′ end of the barcode label does not comprise a blocking moiety and thus can be extended in a polymerase extension reaction when using an adapter barcode oligonucleotide. According to one embodiment, the hybrid of the extended barcode label and the adapter barcode oligonucleotide may be subsequently processed together or separated from each other in order to further continue the method of the first aspect either only with the extended barcode label or with the (preferably extended) adapter barcode oligonucleotide.

According to a preferred embodiment, only the extended barcode label is further processed.

The adapter barcode oligonucleotide can advantageously comprise one or more sequence elements that can be transferred to/incorporated into the extended barcode label via hybridization and barcode extension using the adapter barcode label as template. Such information may comprise one or more of a barcode sequence B_{T} indicating a time information, an UMI sequence, a barcode sequence B_{P} indicating a position information, and a primer sequence (e.g. primer sequence (2)). Particular embodiments of information that can be transferred from the adapter barcode oligonucleotide to the barcode label via hybridization and a polymerase extension reaction are schematically illustrated in FIGS. 7, 8A, 8B and 9. Yet, the disclosure may not be limited to the particular embodiments, and other embodiments including further sequence elements of the barcode label and/or the adapter barcode sequence and/or different arrangements of the barcode label and/or adapter barcode sequence may be applied.

According to one embodiment, step d) comprises (e.g. in step (aa)) adding an adaptor barcode oligonucleotide, wherein the adaptor barcode oligonucleotide comprises an adaptor sequence (1)_{R} that is reverse complementary to an adapter sequence (1) of the barcode label of the detection molecule, wherein the adaptor barcode oligonucleotide additionally comprises at least one, at least two, at least three or all sequence elements selected from the group consisting of a barcode sequence (B_{T}) for indicating a time information, a barcode sequence B_{P} for indicating a position information, a unique molecular identifier (UMI) sequence, and a primer target sequence. These one or more sequence elements are located 5′ of the adaptor sequence (1)_{R} and extending the barcode label using the hybridized adaptor barcode oligonucleotide as template thereby obtaining an extended barcode label. Such an embodiment is schematically illustrated in FIGS. 7, 8A, 8b, and 9.

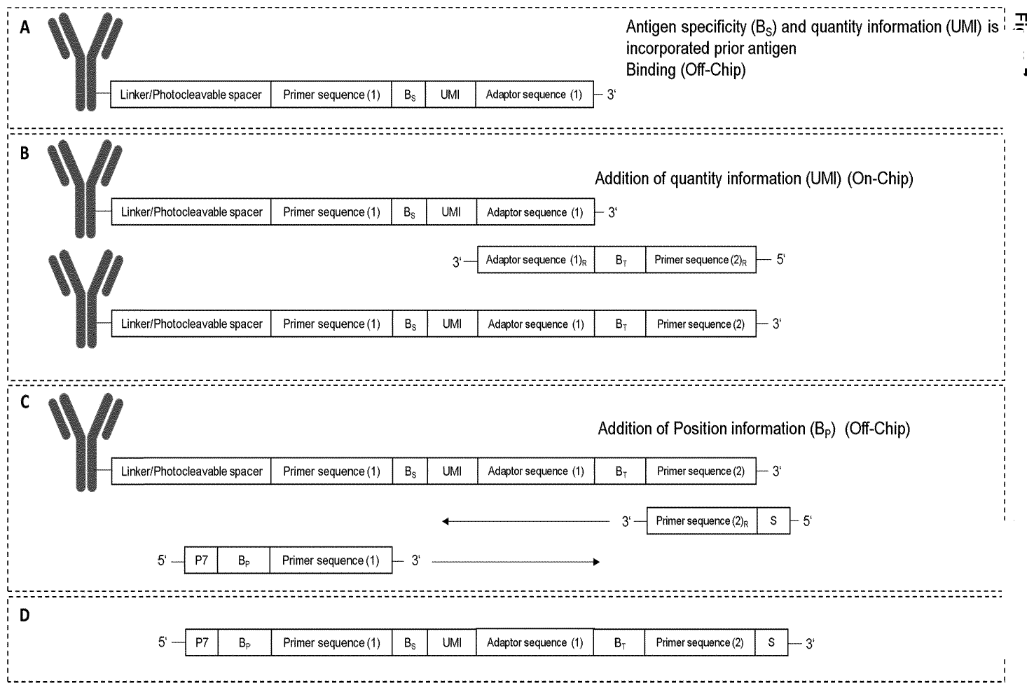

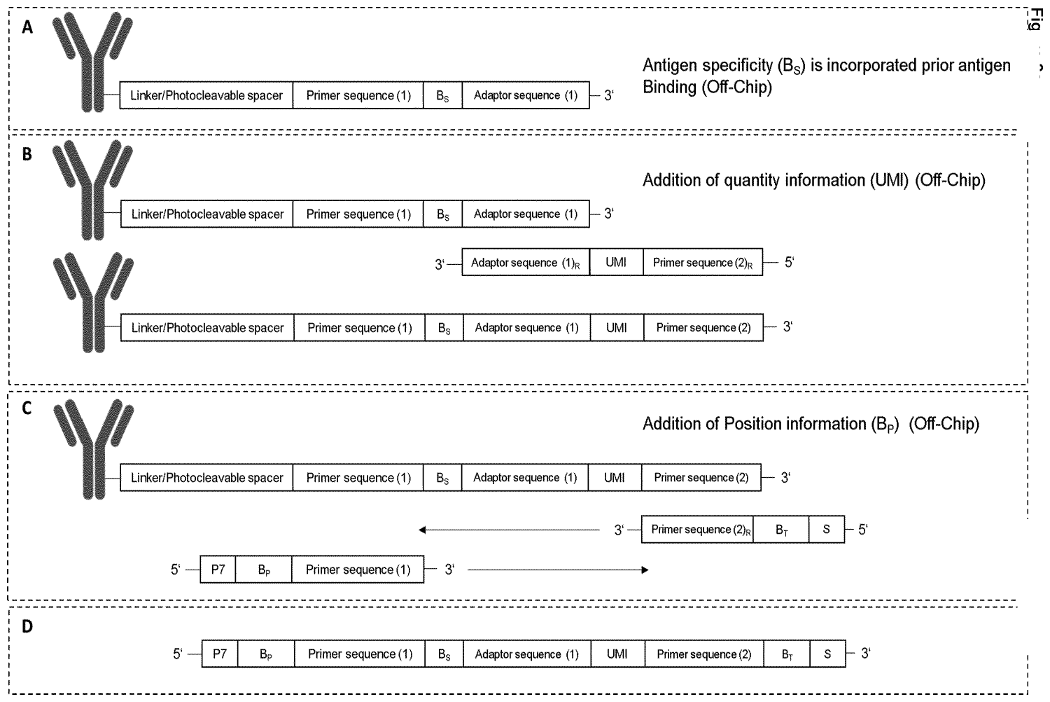

According to a preferred embodiment, the adapter barcode oligonucleotide may comprise an adapter sequence (1)_{R}, which is preferably located at the 3′ end, an UMI sequence, and a primer sequence (2)_{R}, which is preferably arranged at the 5′ end. Other arrangements may also be within the scope of the present disclosure and one or more further sequence elements may be included into such an adapter barcode oligonucleotide. An adapter barcode oligonucleotide comprising the said sequences, may advantageously hybridize to a barcode label at the 3′ end through the hybridization of an adapter sequence (1) comprised in the barcode label (preferably at the 3′ end of the barcode label). The barcode label may advantageously further comprise a barcode sequence B_{S} and a primer sequence (1) at the 5′ end and may be attached to the detection molecule via a linker moiety at the 5′ end (e.g. comprising a photocleavable spacer moiety). Such an embodiment is schematically illustrated in FIG. 8a. After hybridization, the barcode label and optionally, the adapter barcode sequence may be extended, for instance via a polymerase reaction in order to acquire an extended barcode label comprising a primer sequence (1), a barcode sequence B_{S}, an adapter sequence (1), an UMI sequence, and a primer sequence (2). The hybridization reaction and/or the polymerase extension reaction may be performed in the compartment in which the cell-laden matrix is located or at a different position (e.g. storage position, as described above). According to the particular embodiment illustrated in FIG. 8a, both the hybridization reaction and the polymerase extension reaction take place at a different position (also referred to as “Off-Chip”, e.g. a well of a well plate). This advantageously prevents unwanted reactions between the adapter barcode oligonucleotide and/or the polymerase extension reaction components and the cell-laden matrix. According to another embodiment which is depicted in FIG. 9, the adapter barcode sequence additionally comprises a barcode sequence B_{T} can be provided. According to a particular embodiment, the barcode sequence B_{T} is 3′ of the UMI sequence, but may also be 5′ of the UMI sequence. Therefore, in a hybridization and subsequence polymerase extension reaction, not only the UMI sequence and primer sequence (2) but also the barcode sequence B_{T} is present after the polymerase extension on the extended barcode label. According to such an embodiment, the hybridization reaction and optionally, the polymerase extension reaction can take place in the same compartment, wherein the cell-laden matrix is positioned (indicated by being “On-Chip”); however, these may also take place at a different position of the cell culture device (which would also be referred to as “On-Chip”) and/or in a different format (e.g. by transporting the capture matrix to a different format).

According to yet another embodiment, the adapter barcode sequence may comprise an adapter sequence (1)_{R}, preferably at the 3′ end, a barcode sequence B_{T} and a primer sequence (2)_{R}, preferably at the 5′ end. A hybridizing barcode label may preferably comprise a primer sequence (1), preferably at the 5′ end, a barcode sequence B_{S}, an UMI sequence, and an adapter sequence (1), preferably at the 3′ end. Such an embodiment is illustrated in FIG. 7. Thus via hybridization and polymerase extension, the barcode sequence B_{T} and the primer sequence (2) are transferred to/incorporated into the extended barcode label. Furthermore, the adapter barcode sequence may become extended by the polymerase reaction to further comprise sequence elements of the barcode label. According to the particular embodiment, the UMI sequence, the barcode sequence B_{S} and the primer sequence (1)_{R} may become incorporated. Alternatively, in case the 3′ end of the adapter barcode sequence was blocked, it is not elongated by the polymerase elongation reaction. The hybridization reaction may preferably be performed in presence of the cell-laden matrix (e.g. in the compartment of the cell culture device), while the polymerase extension reaction may be performed at a different position (e.g. at a collection position, such as a well of another format). Thereby, advantageously the cell-laden matrix is not contacted with the polymerase extension reaction compounds (e.g. polymerase, dNTPs, etc.).

According to another embodiment, the adapter barcode oligonucleotide may further comprise a barcode sequence B_{P} indicating a position information. Such a barcode sequence B_{P} can be advantageously applied in scope of the present invention in order to provide information about the particular position of the cell-laden matrix/capture matrix, respectively the compartment in which at least one of each matrix are present. The barcode sequence B_{P} can advantageously be applied to incorporate the position information into the extended barcode label, which allows subsequently to pool extended barcode labels (respectively the generated sequenceable reaction product generated by the extended barcode labels as starting material) of different positions and sequence them together in one batch. Afterwards, due to the barcode sequence B_{P} the sequenced signals can be differentiated, allowing for a highly multiplexed analysis. In combination with the barcode sequence B_{S} and optionally the barcode sequence B_{T}, a highly multiplexed analysis can be performed in order to acquire profiles of secreted biomolecules of interest at different positions (and optionally at different time points). According to a preferred embodiment, the barcode label comprises a barcode sequence B_{S} and a primer sequence (1) at the 5′ end and may be attached to the detection molecule via a linker moiety at the 5′ end (e.g. comprising a cleavable spacer moiety). After hybridization, the barcode label and optionally, the adapter barcode sequence may be extended, for instance via a polymerase reaction in order to provide an extended barcode label comprising a primer sequence (1), a barcode sequence B_{S}, an adapter sequence (1), a barcode sequence B_{P}, and preferably an UMI sequence, and a primer sequence (2). The hybridization reaction and/or the polymerase extension reaction may be preferably performed at a different position (e.g. of another format (e.g. a well of a well plate)) than the cell-laden matrix. For instance, the capture matrix (comprising the one or more types of capture molecules, bound biomolecules of interest, thereto bound one or more types of detection molecules comprising the above described barcode label) may be transported to a different collection position such as the well of a well plate, where it is contact with said adapter barcode oligonucleotide. This advantageously prevents unwanted reactions between the adapter barcode oligonucleotide and/or the polymerase extension reaction components and the cell-laden matrix. According to a particular embodiment, the capture matrix may be transported to another compartment, wherein the adapter barcode oligonucleotide, preferably comprising the barcode sequence B_{P}, is immobilized to be released for the hybridization reaction. Immobilization and release may be achieved by methods known in the art. Moreover, the preferred combination of the barcode sequence B_{P} and UMI sequence advantageously reduces the number of required UMI sequences, as the UMI sequences only need to be different for one type of barcode sequence B_{P}. Similar as described above for the barcode sequence B_{T}, UMI sequences may be equal for different positions (i.e. barcode sequences B_{P}). For instance, in case barcode sequence B_{P1} is combined with UMI 1, barcode sequence B_{P2} can also be combined with UMI 1, as the different barcode sequences B_{P} still allows differentiation of signals. According to one embodiment, step d) comprises (aa) adding an adaptor barcode oligonucleotide capable of hybridizing to the barcode label of at least one type of detection molecule, wherein the adaptor barcode oligonucleotide comprises 5′ to the region that is capable of hybridizing to the barcode label (i) a barcode sequence (B_{P}) for indicating a position information and (ii) preferably a unique molecular identifier (UMI) sequence, and extending the barcode label using the hybridized adaptor barcode oligonucleotide as template thereby obtaining an extended barcode label; wherein preferably step d) further comprises (bb) performing an amplification reaction with a primer or primer combination using the extended barcode label and/or the reverse complement thereof as template.

According to another preferred embodiment, an adapter barcode oligonucleotide may be provided comprising an adapter sequence (1)_{R}, a barcode sequence B_{P}, a barcode sequence B_{T}, an UMI sequence, and a primer sequence (2)_{R}. the particular arrangement of these sequence elements may be changed. Preferably, the adapter sequence (1)_{R} is at the 3′ end and the primer sequence (2)_{R} is at the 5′ end. According to such an embodiment, information about the time, position and the UMI can be transferred to the barcode label via hybridization and subsequent polymerase extension, allowing for a multiplexed analysis of different biomolecules of interest (i.e. via different barcode sequences B_{S}), of different time points of cultivation (i.e. via different barcode sequences B_{T}), and of different positions (i.e. via different barcode sequences B_{P}). In addition, the UMI sequence allows to directly quantify the signal and therefore, acquire position-, time-, and biomolecule-dependent information about the biomolecules secreted by at least one cell. Moreover, advantageously, the number of necessary UMI sequences is reduced, as equal UMI sequences can be applied for the barcode labels comprising different barcode sequence B_{P} and B_{T}. Moreover, a subsequent amplification reaction may not require sequence elements particular to the experimental set-up (barcode sequence B_{T}, barcode sequence B_{P}, UMI), rendering the required primer or primer combination simpler.

According to one embodiment, the extended barcode label obtained by hybridizing the adapter barcode oligonucleotide and polymerase extension reaction (or ligation of an oligonucleotide to the barcode label) comprises (i) the barcode sequence (B_{S}) indicating the specificity of the detection molecule, (ii) one or more primer target sequences, (iii) optionally a barcode sequence (B_{T}) indicating a time information and (iv) optionally a unique molecular identifier (UMI) sequence. To allow hybridization of the adapter barcode oligonucleotide to the barcode label, the oligonucleotide furthermore comprises (v) an adapter sequence (1)_{R} that is reverse complementary to a corresponding adapter sequence (1) of the barcode label of the detection molecule to allow hybridization.

According to one embodiment, the barcode sequence B_{T} is provided in the barcode label or the extended barcode label and wherein step d) comprises pooling barcode labels or extended barcode labels provided at different time points and comprising different barcode sequences B_{T} in a compartment prior to performing an amplification reaction. The advantage of such an embodiment is a higher throughput and cost-effectiveness, as multiple capture matrices comprising a barcode label or extended barcode label can be processed, i.e. amplified in the same compartment (e.g. well of a well plate). For example multiple capture matrices of the same position, which were collected subsequently at different time points (indicated by the barcode sequence B_{T}) may be obtained and then together processed by an amplification reaction.

Amplification, Preferably by PCR

An amplification reaction can be conducted apart from or in addition to the above disclosed reaction with the oligonucleotide (also referred to as the adapter barcode oligonucleotide) to extend the barcode label. In case an amplification reaction is performed without prior extension of the barcode label using an oligonucleotide, the capture matrix is after step c) preferably transported to a collection position, not comprising the cell-laden matrix. Such an embodiment allows to keep the at least one cell, unaffected by the amplification reaction. A collection position may be any position wherein an amplification reaction can be conducted. Exemplary position may include another position of the cell culture device, another cell culture device, or another format, such as a well plate (e.g., 96 well plate, 384 well plate, 1536 well plate, etc.) or other reaction vessels (e.g. Eppendorf tube, etc.).

According to one embodiment, an amplification reaction is conducted to generate the sequenceable reaction product. An amplification reaction such as a polymerase chain reaction (PCR) can be performed using a primer combination (e.g. a primer pair), or several cycles of primer extension with a single primer can be performed for amplification. The barcode labels may be released from the detection molecules prior to amplification. Suitable and preferred options for transferring the capture matrix (and/or the released optionally extended barcode labels) are described in detail herein.

The optionally extended barcode label is contacted with a primer or a primer combination (typically comprising a forward and a reverse primer), as well as the reagents required to perform an amplification reaction, such as a polymerase, dNTPs, etc. According to one embodiment, the amplification reaction is performed in the presence of the capture matrix comprising the bound detection molecules. Alternatively, the barcode label/extended barcode label can be removed from the one or more types of detection molecules by cleaving the linker that attaches the barcode label/extended barcode label to the detection molecule. For instance a photocleavable linker may be cleaved by irradiating light with a suitable wavelength, whereupon the barcode label/extended barcode label is cleaved off and remains in solution. Preferably, in case of an extended barcode label, it is released as a single strand. If the adapter barcode oligonucleotide has been extended so as to provide a complementary strand, this strand may be removed prior to amplification. The capture matrix may be removed or the solution comprising the barcode label/extended barcode label may be transferred to another position, wherein the amplification reaction can be performed.

According to one embodiment, the barcode label/extended barcode label is amplified, preferably by PCR. According to one embodiment, the PCR is performed with one or more primers that anneal to the barcode label/extended barcode label. Respective amplification steps are well known in the prior art and thus, do not need any detailed description here.

The Primer(s)

According to one embodiment, a primer or a primer combination hybridizes to a portion of the barcode label (or extended barcode label, depending on of a prior extension polymerization was performed as discussed above). In case one primer is applied, the primer may preferably hybridize to the 3′ end of the barcode label/extended barcode label. Therefore, advantageously the barcode label comprises a primer sequence at the 3′ end complementary to the primer. According to one embodiment, the primer sequence at the 3′ end of the barcode label/extended barcode label is referred to as primer sequence (2) and the primer is reverse complementary to said primer sequence (2), and thus also referred to as primer sequence (2)_{R}. In case a single primer is added to the barcode label/extended barcode label for the amplification reaction, a linear amplification can be performed (e.g. by performing 2 to 20 or 5 to 15 extension cycles with the primer), thereby producing several copies of the reverse strand of the barcode label.

According to another embodiment, a primer combination may be used in order to perform an exponential amplification reaction. A primer combination may advantageously comprise a reverse primer as described above for the linear amplification reaction, and further a forward primer. The forward primer is complementary to the amplified reverse strand and thus advantageously hybridizes thereto and can be extended. According to a preferred embodiment, the forward primer is complementary to the 3′ end of the reverse strand, which comprises a primer sequence. Hence, the forward primer comprises a primer sequence which is identical to a primer sequence, which can be advantageously provided by the barcode label. According to a preferred embodiment, the barcode label comprises a primer sequence, referred to as primer sequence (1) at the 5′ end (also described above in conjunction with the barcode label) and the forward primer comprises a primer sequence which is identical to the primer sequence (1). Therefore, according to a preferred embodiment, the reverse primer is capable of hybridizing to the barcode label/extended barcode label to generate the complementary strand, whereupon in the de- and rehybridization cycle an amplification reaction can be performed of the forward primer hybridized to the formed complementary/reverse strand (comprising the primer sequence (1)_{R}). Hence, in the next polymerization reaction, the forward strand is polymerized.

According to one embodiment, the adapter barcode oligonucleotide is extended by the polymerase extension disclosed above. In case the extended adapter barcode oligonucleotide is amplified, it may be sufficient to provide the above described forward primer to perform a linear amplification or to provide a primer combination of said forward and reverse primer for an exponential amplification. Complementary considerations as disclosed above can be applied in such an embodiment.

According to a preferred embodiment, the one or more primers comprise one or more sequence elements, which can be amplified in an amplification reaction to be incorporated into the generated sequenceable reaction product. Typically, the primer or primer combination comprise—apart from the complementary primer sequences capable of hybridizing to the barcode label/extended barcode label—one or more further sequence elements that are not present in the barcode label/extended barcode label. For instance, the barcode label comprises a barcode sequence B_{S} and thus preferably the one or more primers do not comprise a barcode sequence B_{S}. Similar consideration may be applied, in case the barcode label/extended barcode label comprises a barcode sequence B_{T} and/or B_{P}, wherein the primer or primer combination does then not require such sequence elements. Yet, it is also within the scope of the present invention to provide redundancy if required and thus provide one or more sequence elements more than one time (e.g. in the barcode label/extended barcode label and the primer or primer combination). Such a sequence redundancy may be used to improve the signal strength.

According to a preferred embodiment, the primer sequence only comprises such further sequences that are not already provided by the barcode label/extended barcode label (apart from the primer sequences which need to be present in order to hybridize to the template and perform an amplification reaction). For example, if the barcode label/extended barcode label comprises a barcode sequence B_{S} and a barcode sequence B_{T}, it may be advantageous to add a barcode sequence B_{P} to the barcode label/extended barcode label via the one or more primers. According to another example, if the barcode label/extended barcode label comprises a barcode sequence B_{S} and a barcode sequence B_{P}, it may be advantageous to add a barcode sequence B_{T} to the barcode label/extended barcode label via the one or more primers. It may not be desirable to add an UMI sequence via the one or more primers, as the UMI sequence is preferably unique for each detection molecule and thus would be less specifically copied in an amplification reaction. However, in some cases it may be required and it is not excluded by the present disclosure that UMI sequences can also be present in the one or more primers.

According to a preferred embodiment, the generated sequenceable reaction product comprises sequencing adapter sequences (AS) at the 3′ and 5′ ends in order to be accessible for a sequencing reaction. Adapter sequences are well known in the art and the present disclosure may not be limited to particular sequencing adapters. Exemplary adapter sequences may be “P7” and “S”. It is not required though to include such adapter sequences in view of the present disclosure, as these can also be subsequently attached to the generated sequenceable reaction product via methods known in the art. According to a preferred embodiment, such adapter sequences are provided in the sequenceable reaction product in order to generate a product that can be sequenced time and cost efficiently. The adapter sequences can be comprised in the barcode label, in the adapter barcode sequence (in order to be transferred to the barcode label in form of the extended barcode label), or may be added to the sequenceable reaction product via the primer or primer combination. Preferably, if a primer combination is applied, each primer comprises at the 5′ end an adapter sequence. In case one primer is applied for linear amplification, it may preferably comprise one adapter sequence at the 5′ end, whereas the barcode label/extended barcode label comprises the second adapter sequence at the 5′ end.

According to a preferred embodiment, the sequenceable reaction product is generated by amplifying the barcode label or extended barcode label in an amplification reaction. Therefore, one or more primers (e.g. one primer for a linear amplification or a primer combination for exponential amplification) can be applied. In case, a single primer is applied, the primer may preferably be reverse complementary to 3′ end of the optionally extended barcode label (i.e. the barcode label comprises a complementary primer sequence at the 3′ end). For instance the optionally extended barcode label may comprise a primer sequence (2) at the 3′ end, which is reverse complementary to the primer, which comprises a primer sequence (2)_{R}. Hence, in an amplification reaction, the primer can hybridize to the barcode label to form a template for a polymerase, which can then advantageously form the complementary/reverse strand. Preferably, the primer further comprises an adapter sequence (e.g. “S”) at the 5′ end to be utilized in a later sequencing. In such a case, the 5′ end of the barcode label preferably comprises another adapter sequence at the 5′end for later sequencing.

According to a preferred embodiment, a primer combination is used, which, in addition to said reverse primer, comprises a forward primer that comprises an identical sequence as the barcode label (preferably an identical sequence of the 5′ end of the barcode label). For instance, the barcode label may comprise a primer sequence (1) at the 5′ end and the forward primer comprises also a primer sequence (1). In frame of an amplification reaction, the reverse primer first amplifies the reverse strand of the barcode label. In a second amplification reaction, the forward primer then hybridizes to the reverse strand, in particular to the primer sequence (1)_{R} and then serves as a template for a polymerase reaction. The forward primer preferably comprises an adapter sequence at the 5′ end in order to generate a sequenceable reaction product that can be directly sequences without requiring attachment of adapter sequences. For instance, the forward primer may comprise an adapter sequence P7. Hence, a sequenceable reaction product generated inter alia by the primer combination advantageously comprises at the 5′ and the 3′ end an adapter sequence and is amplified in an exponential manner to provide multiple copies of the sequenceable reaction product.

Particular Primer Embodiments

The optionally extended barcode label comprises a primer sequence or two primer sequences to which the primer or a primer of the primer combination, respectively, can hybridize to initiate the amplification reaction.

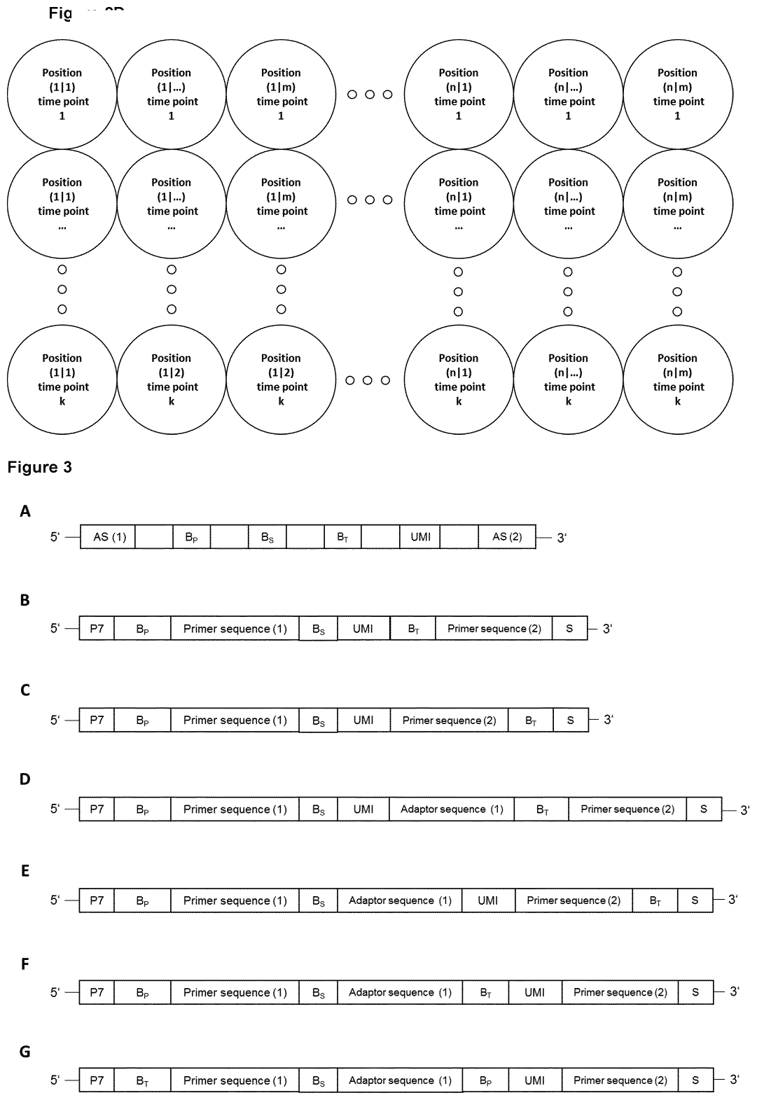

According to a particular embodiment, which is also schematically illustrated in FIG. 5, the barcode sequence B_{P} is introduced into the sequenceable reaction product via an oligonucleotide that is used in step d), wherein the oligonucleotide comprising the barcode sequence B_{P} is a primer that is used in an amplification reaction. For instance, the (optionally extended) barcode label may comprise a primer sequence (1) at the 5′ end, a primer sequence (2) at the 3′ end, as well as a barcode sequence B_{S}, an UMI sequence and a barcode sequence B_{T} (from 5′ to 3′). The primer combination may comprise a reverse primer comprising a primer sequence (2)_{R}, preferably at the 3′ end, and a sequencing adapter sequence (here “S”) at the 5′ end and a forward primer comprising a primer sequence (1) at the 3′ end, an adapter sequence (here “P) at the 5′ end and a barcode sequence B_{P}. During an amplification reaction, the sequencing adapter sequences and the barcode sequence B_{P} are transferred to/incorporated into the generated sequenceable reaction product, which afterwards comprises the adapter sequences (at the 3′ and 5′ end), the barcode sequence B_{P}, the primer sequence (1), the barcode sequence B_{S}, the UMI sequence, the barcode sequence B_{T}, and the primer sequence (2) (see also FIG. 3B). Such an embodiment may be advantageous, as it does not require a polymerization extension step to transfer sequence elements from an adapter barcode oligonucleotide to the barcode sequence but the required information is present in the barcode label and the primer combination. The amplification reaction may preferably be performed at a position not comprising the cell-laden matrix in order to not affect the at least one cell of the cell-laden matrix. Thus the amplification reaction may preferably be performed at a collection position (e.g. a well of a well plate). It may furthermore be advantageous to perform such an amplification reaction at a different position in order to incorporate the barcode sequence B_{P} into the sequenceable reaction product. Other configurations of the particular embodiment are also applicable in scope of the present disclosure. For example, the arrangement of sequence elements (here B_{S}, UMI and B_{T}) may differ. Moreover, the barcode sequence B_{T} may be provided by the one or more primers.

According to one embodiment, step d) comprises performing an amplification reaction with a primer or primer combination comprising
a barcode sequence (B_{P}) for indicating position information,
optionally an adapter sequence (AS) for sequencing,
optionally a barcode sequence (B_{T}) for indicating a time information.

The one or more sequence elements B_{P}, AS, and/or B_{T}, if included in the primer or the primer combination, are located 5′ of the sequence region of the primer that is capable of hybridizing to the optionally extended barcode label or the reverse complement thereof. The barcode sequence B_{T} is in particular provided by the primer of the primer combination in case the barcode (B_{T}) is not comprised in the barcode label (or the optionally extended barcode label). A particular embodiment is schematically illustrated in FIG. 6. In that particular example, the barcode label comprises a primer sequence (1), a barcode sequence B_{S}, an UMI sequence, and a primer sequence (2) (from 5′ to 3′). In such an embodiment, a primer combination may be advantageously applied that adds the barcode sequence B_{T} and/or barcode sequence B_{P} to the generated sequenceable reaction product. According to a preferred embodiment, both barcode sequence (B_{P} and B_{T}) are added to the sequenceable reaction product by the primer combination. In a particular embodiment, a primer combination may be advantageously applied, wherein one primer, preferably the reverse primer, comprises a primer sequence (2)_{R} at the 3′ end, a barcode sequence B_{T} and an adapter sequence (e.g. “S”) at the 5′ end, and another primer, preferably the forward primer comprises a primer sequence (1) at the 3′ end, a barcode sequence B_{P} and an adapter sequence (e.g. “P7”) at the 5′ end. Other configurations of the forward and reverse primer may be applied in scope of the embodiment, such as for instance a reverse or forward primer comprising both the barcode sequence B_{T} and B_{P} or the barcode sequences B_{P} and B_{T} may be exchanged (e.g. barcode sequence B_{T} may be provided in the forward primer and barcode sequence B_{P} may be provided in the reverse strand).

According to one embodiment, the generation of the sequenceable reaction product comprises the use of at least one oligonucleotide, optionally a primer, that is capable of hybridizing to the barcode label of the at least one type of detection molecule. According to one embodiment, step d) comprises (aa) hybridizing at least one oligonucleotide to the barcode label of at least one type of detection molecule and extending said barcode label using the hybridized oligonucleotide as template thereby obtaining an extended barcode label attached to the detection molecule that additionally comprises sequence information of the hybridized oligonucleotide that was used as template, optionally wherein step d) further comprises (bb) performing an amplification reaction with a primer or primer combination using the extended barcode label and/or the reverse complement thereof as template, wherein preferably, the extended barcode label is used as template.

According to one embodiment, where at least one oligonucleotide is hybridized to the barcode label of at least one type of detection molecule and is extended in a step (aa) of step d), using the hybridized oligonucleotide as template to thereby obtain an extended barcode label attached to the detection molecule that additionally comprises one or more sequence elements of the hybridized oligonucleotide that was used as template, a step (bb) of step d) may be performed by performing an amplification reaction with a primer or primer combination using the extended barcode label and/or the reverse complement thereof as template. The at least one oligonucleotide that is capable of hybridizing to the barcode label of the at least one type of detection molecule to which claim 1 refers may correspond in these embodiments to the oligonucleotide (also referred to as adaptor barcode oligonucleotide) that is capable of hybridizing to the barcode label. In the case the method of the first aspect makes use of an oligonucleotide, also referred to as an adapter barcode sequence, an extended barcode label is preferably generated. Such extended barcode labels have been described in detail above and also apply here. Preferably the extended barcode label comprises one or two primer sequences, which can be provided in the original barcode label (non-extended), preferably at the 5′ end (e.g. as primer sequence (1)) and/or can be introduced via the complementary sequence of the primer sequence (e.g. primer sequence (2)_{R}) of the adapter barcode oligonucleotide, preferably at the 3′ end. Preferably, the extended barcode sequence comprises a primer sequence (1) at the 5′ end and a primer sequence (2) at the 3′ end.

According to one embodiment, step d) comprises (aa) adding an adaptor barcode oligonucleotide capable of hybridizing to the barcode label of at least one type of detection molecule, wherein the adaptor barcode oligonucleotide comprises 5′ to the region that is capable of hybridizing to the barcode label a unique molecular identifier (UMI) sequence, and extending the barcode label using the hybridized adaptor barcode oligonucleotide as template thereby obtaining an extended barcode label. Step d) preferably further comprises (bb) performing an amplification reaction with a primer or primer combination using the extended barcode label and/or the reverse complement thereof as template.

According to one embodiment, step d) comprises (aa) adding an adaptor barcode oligonucleotide capable of hybridizing to the barcode label of at least one type of detection molecule, wherein the adaptor barcode oligonucleotide comprises 5′ to the region that is capable of hybridizing to the barcode label (i) a barcode sequence (B_{T}) for indicating a time information and/or (ii) a unique molecular identifier (UMI) sequence, and extending the barcode label using the hybridized adaptor barcode oligonucleotide as template thereby obtaining an extended barcode label; wherein preferably step d) further comprises (bb) performing an amplification reaction with a primer or primer combination using the extended barcode label and/or the reverse complement thereof as template. Exemplary embodiments are schematically illustrated in FIGS. 7, 8A and 9 and it is here referred thereto (including the Figure descriptions).

According to a particular embodiment, the extended barcode label may comprise a primer sequence (1), a barcode sequence B_{S}, an UMI sequence, an adapter sequence (1), a barcode sequence B_{T} and a primer sequence (2) (preferably from 5′ to 3′). The extended barcode label has been discussed above and it referred thereto for further details. In the substep (bb) of that particular embodiment, i.e. the amplification step, a single primer or a primer combination may be applied. Preferably a primer combination may be applied, which additionally provides a barcode sequence B_{P} and adapter sequences at the 3′ and 5′ end to the generated sequenceable reaction product. In a particular embodiment, the reverse primer may comprise a primer sequence (2)_{R} which is reverse complementary to the primer sequence (2), preferably provided at the 3′ end of the extended barcode label. Moreover the reverse primer may advantageously provide a sequencing adapter sequence (e.g. “S”) at the 5′ end. The forward primer may comprise a primer sequence (1) at the 3′ end which comprises an identical sequence to the primer sequence (1) provided by the extended barcode label (preferably at the 5′ end). Moreover, the forward primer may comprise a barcode sequence B_{P} and an adapter sequence (e.g. “P7”) at the 5′ end. After amplification, for instance in a PCR reaction of the extended barcode label with said primer combination, a sequencebale reaction product may be generated which is schematically illustrated in FIG. 3D. The applied primer(s) may be configured differently and shall not be limited to the particular arrangement (e.g. the reverse primer may comprise the barcode sequence B_{P}). In case a single primer is applied, preferably, the primer comprises a primer sequence (2)_{R} at the 3′ end and a barcode sequence B_{P}, as well as an adapter sequence (e.g. “S” or P7”). In such an embodiment, the extended barcode label preferably comprises an adapter sequence at the 5′ end in order to generate a sequenceable reaction product that can be directly applied for sequencing. If only a single primer is used for amplification by performing several cycles of primer extension, it is not required to provide a primer sequence (1) in the barcode label. As described herein, in case a single primer is applied, a linear amplification reaction can be performed. It follows from the above disclosure that the arrangement of the sequence elements B_{P}, B_{S}, UMI and B_{T} may vary depending on the used embodiment. E.g., the barcode sequence B_{P} may be located between the primer sequence (2) and the adapter sequence (e.g. “S”), the order of the barcode sequence B_{S} and UMI sequence may be reversed and the primer sequence (1) may be missing, if only a single primer is used for amplification.

According to a particular embodiment, the extended barcode label may comprise a primer sequence (1), a barcode sequence B_{S}, an adapter sequence (1), an UMI sequence, and a primer sequence (2) (preferably from 5′ to 3′). The extended barcode label may be attached to the corresponding detection molecule via a linker moiety (preferably a photocleavable linker). The extended barcode label has been discussed above and it referred thereto for further details. In the sub-step (bb) of that particular embodiment, i.e. the amplification step, a single primer of a primer combination may be applied. Preferably a primer combination may be applied, which additionally provides a barcode sequence B_{P} and/or a barcode sequence B_{T} (preferably both barcode sequences) and adapter sequences at the 3′ and 5′ end to the generated sequenceable reaction product. In a particular embodiment, the reverse primer may comprise a primer sequence (2)_{R} which is reverse complementary to the primer sequence (2), preferably provided at the 3′ end of the extended barcode label. Moreover the reverse primer may advantageously provide a barcode sequence B_{T} and an adapter sequence (e.g. “S”) at the 5′ end. The forward primer may comprise a primer sequence (1) at the 3′ end which comprises an identical sequence to the primer sequence (1) provided by the extended barcode label (preferably at the 5′ end). Moreover, the forward primer may comprise a barcode sequence B_{P} and an adapter sequence (e.g. “P7”) at the 5′ end. After amplification, for instance in a PCR reaction of the extended barcode label with said primer combination, a sequenceable reaction product may be generated which is schematically illustrated in FIG. 3E. The applied primer(s) may be configured differently and shall not be limited to the particular arrangement (e.g. the reverse primer may comprise the barcode sequence B_{P} and the forward primer may comprise the barcode sequence B_{T} or one or the primers may comprise both barcode sequences B_{T} and B_{P}). In case only a single primer is applied, preferably, the primer comprises a primer sequence (2)_{R} at the 3′ end and a barcode sequence B_{P} and a barcode sequence B_{T}, as well as an adapter sequence (e.g. “S” or P7”). In such an embodiment, the extended barcode label preferably comprises an adapter sequence at the 5′ end in order to generate a sequenceable reaction product that can be directly applied for sequencing. If only a single primer is used for amplification by performing several cycles of primer extension, it is not required to provide a primer sequence (1) in the barcode label, as described herein. It follows from the above disclosure that the arrangement of the sequence elements B_{P}, B_{S}, UMI and B_{T} may vary depending on the used embodiment. E.g., the barcode sequence B_{P} may be located between the primer sequence (2) and the adapter sequence (e.g. “S”), the order of the barcode sequence B_{S} and UMI sequence may be reversed and the primer sequence (1) may be missing, if only a single primer is used for amplification.

According to a particular embodiment, the extended barcode label may comprise a primer sequence (1), a barcode sequence B_{S}, an adapter sequence (1), a barcode sequence B_{T}, an UMI sequence and a primer sequence (2) (preferably from 5′ to 3′). The extended barcode label may be attached to the corresponding detection molecule via a linker moiety (preferably a photocleavable linker). The extended barcode label has been discussed above and it referred thereto for further details. In the substep (bb) of that particular embodiment, i.e. the amplification step, a single primer of a primer combination may be applied. Preferably a primer combination may be applied, which additionally provides a barcode sequence B_{P} and adapter sequences at the 3′ and 5′ end to the generated sequenceable reaction product. In a particular embodiment, the reverse primer may comprise a primer sequence (2)_{R} which is reverse complementary to the primer sequence (2), preferably provided at the 3′ end of the extended barcode label. Moreover the reverse primer may advantageously provide an adapter sequence (e.g. “S”) at the 5′ end. The forward primer may comprise a primer sequence (1) at the 3′ end which comprises an identical sequence to the primer sequence (1) provided by the extended barcode label (preferably at the 5′ end). Moreover, the forward primer may comprise a barcode sequence B_{P} and an adapter sequence (e.g. “P7”) at the 5′ end. After amplification, for instance in a PCR reaction of the extended barcode label with said primer combination, a sequencebale reaction product may be generated which is schematically illustrated in FIG. 3F. The applied primers may be configured differently and shall not be limited to the particular arrangement (e.g. the reverse primer may comprise the barcode sequence B_{P}). In case only a single primer is applied, preferably, the primer comprises a primer sequence (2)_{R} at the 3′ end and a barcode sequence B_{P}, as well as an adapter sequence (e.g. “S” or P7”). In such an embodiment, the extended barcode label preferably comprises an adapter sequence at the 5′ end in order to generate a sequenceable reaction product that can be directly applied for sequencing. Again, only a single primer may be used for amplification by performing several cycles of primer extension as is described herein. The arrangement of the sequence elements B_{P}, B_{S}, UMI and B_{T} may vary depending on the used embodiment. E.g., the barcode sequence B_{P} may be located between the primer sequence (2) and the adapter sequence (e.g. “S”), the order of the barcode sequence B_{S} and UMI sequence may be reversed and the primer sequence (1) may be missing, if only a single primer is used for amplification.

According to a preferred embodiment, the generation of the sequenceable reaction product in step d) comprises the use of (i) at least one oligonucleotide, optionally a primer, and/or (ii) a primer combination, wherein the at least one oligonucleotide and/or the primer combination includes one or more sequence elements selected from the group consisting of
a barcode sequence (B_{T}) for indicating a time information,
a barcode sequence (B_{P}) for indicating position information of a cell-laden matrix,
a unique molecular identifier (UMI) sequence, and
an adapter sequence (AS) for sequencing,

wherein the one or more sequence elements B_{T}, B_{P}, UMI and/or AS, if included, are located 5′ of the sequence region of the oligonucleotide and/or primer that is capable of hybridizing to the barcode label of the detection molecule or the reverse complement thereof.

According to one embodiment, at least one primer of the primer combination, which preferably is a pair, is the same for all extension products comprised in the different compartments of the device.

The UMI barcode provided in the sequenceable product may have a length of up to 40 nucleotides, preferably 4-20 nucleotides. A length of 40 base pairs is sufficient to label e.g. up to one mole detection molecules, and hence up to one mole captured biomolecules of interest. Before amplification of the UMI barcodes, each UMI barcode occurs only one in each capture matrix.

Compartments and Cell Culture Device

According to a preferred embodiment, steps a) to c) and step d) either entirely or in part are performed utilizing a cell culture device (e.g. a microfabricated device), which has been described above and is further disclosed below in the further embodiments of the method of the first aspect, which apply also here. For instance, step a) to d) may be performed entirely utilizing a cell culture device, wherein in step d) the use of (i) at least one oligonucleotide, optionally a primer, and/or (ii) a primer combination, may be conducted in a compartment that is different from the compartment, wherein the cell-laden matrix is incubated, optionally in presence of the capture matrix. According to one embodiment, the use of at least one oligonucleotide, which may be an adapter barcode oligonucleotide is performed in the same compartment as the incubation of the cell culture device, whereupon the capture matrix may either be transferred to a storage position or a polymerase reaction is performed in the compartment comprising the cell-laden matrix to extend the barcode label (e.g. generating an extended barcode label). Such an embodiment has the advantage that the process can be performed on the cell culture device in a multiplexed manner. Alternatively, after using the adapter barcode oligonucleotide and hybridizing said oligonucleotide to the barcode label, the capture matrix may be transferred to a storage/collection position. As described above, the storage position may be any position capable of storing/holding one or more capture matrices (e.g. including a well of a well plate or another compartment of the cell culture device, wherein the device does not comprise the cell-laden matrix). At the storage position, the barcode label hybridized to the adapter barcode oligonucleotide may be extended by a polymerase reaction. In addition or alternatively, a single primer or a primer combination may be used in order to generate a sequenceable reaction product.

According to one embodiment, the addition of sequence elements selected from the group consisting of
a barcode sequence (BT) for indicating a time information,
a barcode sequence (BP) for indicating position information of a cell-laden matrix,
a unique molecular identifier (UMI) sequence, and
an adapter sequence (AS) for sequencing

is performed after obtaining the capture matrix and transferring the capture matrix to a storage position. In a particular example, the capture matrix may be obtained after step c) and transferred to a well of a well plate, wherein step d) is performed, preferably by using a polymerase extension and/or amplification reaction. In such an example, the position information can be incorporated by the single primer or primer combination or by an adapter barcode oligonucleotide. Therefore, it is also applicable in scope of such an example, to obtain multiple capture matrices after step c), e.g. from different time points but same positions and transfer said capture matrices in the same storage position (e.g. same well of a well plate). Thus, advantageously, the PCR reaction can be pooled for a cost effective generation of sequenceable reaction products (e.g. by saving primer, reagent, enzyme, etc.).

According to one embodiment, the capture matrix can also be transferred to a storage position after step b), wherein further steps c) and d) may be performed. It may be preferred to perform at least step c) in a cell culture device or similar device in order to process the capture matrices in a multiplexed manner.

According to one embodiment, at least one cycle of steps a) to d) is performed for a plurality of cell-laden matrices comprised in different compartments and wherein the sequenceable reaction product that is generated in step d) comprises a barcode sequence B_{P} for indicating position information of a cell-laden matrix analysed, wherein a sequenceable reaction product is generated for a cell-laden matrix comprised in a compartment that differs in its barcode sequence B_{P} from the barcode sequence B_{P} of the sequenceable reaction product(s) generated for a cell-laden matrix comprised in another compartment.

According to one embodiment, the templates comprised in different compartments of a device are contacted with a different subtype of the primer or primer combination, wherein the different subtypes of the primer or primer combination differ in their barcode sequence B_{P} that indicates the position information of an individual compartment, wherein preferably, the subtypes of the primer or primer combination are identical except for the barcode sequence B_{P} that is unique for each subtype.

According to one embodiment, the amplification in step d) is performed by contacting the templates comprised in different compartments of a device with different primer combinations, wherein one primer of the primer combination is the same for all templates comprised in different compartments of the device and the other primer of the primer combination differs in the barcode sequence B_{P} that indicates the position information of an individual compartment. Preferably, the primer combinations provided in the different compartments is identical, except for the barcode sequence B_{P} that is unique for each compartment (i.e. subtype).

Moreover, it may not even be required in such an embodiment to provide a barcode sequence B_{P} as the capture matrices are stored in particular storage positions, which is different for capture matrices of different positions (e.g. of different compartments). In such an embodiment, pooling of generated sequenceable reaction products from capture matrices of different positions may not be performed but the sequenceable reaction products may be sequenced separately. Advantageously, the single primer sequence or primer combination does not need to comprise a barcode sequence B_{P} in such a case. According to another embodiment, it may not be required to provide a barcode sequence B_{P} to be incorporated into the generated sequenceable reaction product, in case multiple capture matrices of the same reaction conditions (e.g. same cell type, same reaction conditions, same incubation time), which may also be referred to as replica, are obtained to be analyzed. In such a case, the mean value (e.g. molecule number, concentration) of analyzed biomolecules of interest (e.g. through the sequencing of the sequenceable reaction product) can be obtained. Such an embodiment may be useful to increase the throughput of the disclosed method.

According to one embodiment, the sequenceable reaction product comprises a barcode sequence (B_{T}) for indicating a time information and wherein n cycles of steps a) to c) and optionally step d) are performed at different time points tx, wherein n is at least 2 and x indicates the different time points, and wherein for each cycle a sequenceable reaction product is generated that differs in its barcode sequence B_{T} from the barcode sequence B_{T} of all other performed cycles. Such an embodiment advantageously comprises obtaining more than one capture matrix from the same compartment repeatedly. In particular, a capture matrix may be incubated together with the cell-laden matrix for a particular time interval. At the time point t₁, the capture matrix may be obtained from the compartment (e.g. after at least performing step c), optionally, further performing step d) in part) and transferred to a storage position, whereas the cell-laden matrix remains inside the compartment. Furthermore another capture matrix (also referred to as new capture matrix or “fresh” capture matrix), can be positioned next to the remaining cell-laden matrix inside the compartment. Released biomolecules may be repeatedly bound by the capture molecules of the capture matrix, which advantageously allows to acquire time-lapse secretion profiled of biomolecules of interest. According to one embodiment, the steps a) to c) are performed more than one time. Therefore, the capture matrix is in step c) preferably transferred after a defined time interval, which is further disclosed below and also applies here, into a storage position and a “fresh” capture matrix is transferred into the compartment comprising the cell-laden matrix. Therefore, advantageously the disclosed reverse flow cherry picking may be applied. The steps may be performed more than one time, preferably ≥two times, ≥three times, ≥four times, more preferably ≥five times. According to one embodiment, step d) is performed repeatedly after step c), in particular after the transfer of the capture matrix or multiple capture matrices of the more than one cycle are collected and step d) is performed of all transferred capture matrices. In case an adapter sequence oligonucleotide is applied in step d), capture matrices may be collected together after hybridization and polymerization (referred to as step (aa) of step d)), which have been further disclosed above and said disclosures also apply here. Afterwards, the capture matrices of different cycles may be combined to perform step d), in particular step (bb) of step d).

The sequenceable reaction product comprising different barcode sequences (B_{T}) for indicating a time information as disclosed herein can also be obtained using a cell culture device which is a cell culture plate, such as a 12-well plate, a 24-well plate, 96-well plate, a 384-well plate. Such an embodiment advantageously comprises obtaining more than one capture matrix from the same compartment of the cell culture plate, e.g. well. In line, at the time point t₁, the capture matrix may be obtained from the compartment of the cell culture plate, e.g., well, and optionally transferred, whereas the cell-laden matrix remains inside the compartment. Furthermore, a fresh capture matrix can be added to the remaining cell-laden matrix inside the compartment of the cell culture plate. Released biomolecules may be repeatedly bound by the capture molecules of the capture matrix, which advantageously allows to acquire time-lapse secretion profiled of biomolecules of interest. Incubation of the cell-laden matrix in the compartment of the cell culture plate can take place for a time interval selected from ≥10 min, ≥20 min, ≥30 min, ≥1 h, ≥2 h, ≥3 h, ≥4 h, 5 h or more, up to days 1 d, 2 d or several days. According to one embodiment, the time interval is selected from the range of 30-120 min. The repeated incubation and binding can be performed multiple times, e.g. ≥two times, ≥three times, ≥four times, more preferably five times.

Step e)

According to a preferred embodiment, a step e) is performed, which comprises sequencing the generated sequenceable reaction product(s).

The method may comprise pooling sequenceable reaction products generated in step d) from different cycles and/or generated from different compartments and sequencing the obtained pool. According to one embodiment, a plurality of sequenceable reaction products (e.g. an oligonucleotide library) is generated comprising a library of different barcode labels (e.g. oligonucleotides) that contain a barcode sequence B_{S}, a barcode sequence B_{T} and/or a barcode sequence B_{P}, and optionally a quantity information (UMI), as well as optionally, adapter sequences for sequencing. In such a case, the sequenceable reaction product can advantageously encode the required information to correlate the sequencing results to the respective at least one cell (barcode sequence B_{P}), the time point (barcode sequence B_{T}), the biomolecules of interest (barcode sequence B_{S}) and the quantity of biomolecules of interest (UMI sequence). In such a case the generated sequenceable reaction products can be pooled (e.g. a defined fluid volume is taken from each generated sequenceable reaction product and put together, e.g. in one reaction tube) and sequenced/sent for sequencing. Hence, a few or even a single NGS samples may be obtained for sequencing, which is cost effective, as it saved the amount of required sequencing components (e.g. primer, reagents, enzymes, etc.). Further embodiments are described herein.

Prior to sequencing, it is advantageous to perform an amplification reaction to acquire multiple copies of the optionally extended barcode label. Accurate quantification is still possible if UMI sequences are used.

Details of the plurality of sequenceable reaction products that can be provided using the method according to the first aspect are also disclosed in conjunction with the third aspect of the present invention.

The generated sequenceable reaction product(s), which may be preferably pooled, can be sequenced using any sequencing method. In case further steps to modify the sequenceable reaction product (e.g. by addition of particular adapters) are required in order to be sequenceable by a particular sequencing technologies, these may either be performed throughout step d) of the presently disclosed method or may be subsequently performed in frame of the sequencing protocol of the applied sequencing technology. The disclosed method may not be limited by the particularly applied sequencing technology.

Preferably, sequencing is performed by next generation sequencing (NGS). Prior art next-generation sequencing approaches are reviewed e.g. in Goodwin et al., Nature Reviews, June 2016, Vol. 17: pp. 333-351 “Coming of age: ten years of next-generation sequencing technologies”, Yohe et al., Arch Pathol Lab Med, November 2017, Vol. 141: pp. 1544-1557 “Review of Clinical Next-Generation Sequencing” and Masoudi-Nejad, Chapter 2 “Emergence of Next-Generation Sequencing in “Next Generation Sequencing and Sequence Assembly” SpringerBriefs in Systems Biology, 2013, all herein incorporated by reference. Widely used and here applicable sequencing approaches are referred to such as sequencing by synthesis (SBS) and sequencing by ligation (SBL). SBS includes following non-limiting sequencing technologies: cyclic reversible termination (e.g. Illumina, QIAGEN) and single-nucleotide addition (IonTorrent). SBL includes following non-limiting sequencing technologies: SOLiD and complete Genomics. Non limiting, further applicable sequencing technologies include DNA microarrays, Nanostring, qPCR, optical mapping, single-molecule real-time (SMRT) sequencing (e.g. Pacific Biosciences), Oxford Nanopore Technologies.

Step f), Analyzing the Obtained Sequencing Data

The method may furthermore comprise a step f), comprising evaluating the obtained sequencing data. The analysis of the sequencing data can advantageously be performed to correlate the obtained sequencing data with information about the one or more biomolecules of interest.

In particular, the analysis of the obtained sequencing data based on the core sequence elements described herein allows to correlate the obtained sequencing data with the type of released biomolecule of interest (e.g. by the sequencing data obtained from the B_{S} sequence element). Moreover, a further correlation can be drawn on the number of biomolecules of interest released (e.g. by the sequencing data obtained from the UMI sequence element), the time point of cultivation at which the capture matrix was obtained (e.g. by the sequencing data obtained from the B_{T} sequence element), and/or the position of the compartment, respectively the position of the cell-laden matrix and/or the capture matrix (e.g. by the sequencing data obtained from the B_{P} sequence element). The number of further correlations that can be drawn depend on the sequence elements that were incorporated into the generated sequenceable reaction product. The results may be plotted in form of a diagram, e.g. quantity of the one or more biomolecules of interest over time, for the different positions/cell-laden matrices.

As disclosed herein, the generated sequenceable reaction products may be pooled in order to generate a pooled sequenceable reaction product for sequencing. The differentiation of the sequenceing reaction products comprised in the pool into the individual sequencing reaction products may be performed on the basis of the sequence elements which are incorporated into the sequenceable reaction products of the individual sequencing reaction products. Thus, advantageously, based on the sequencing data, an analysis algorithm can be employed to extract the sequencing information and to determine on such a basis the concentration/number of the biomolecules of interest. The method of analysis (which may also be referred to as an analysis algorithm) can vary depending on the provided sequence elements incorporated into the generated sequenceable reaction product and depending on the generated pool. Ideally, the method of analysis should be capable of differentiating the individually captured biomolecules of interest by the sequencing information of the (pooled) sequenceable reaction products.

According to one embodiment, the analysis algorithm first identifies and categorizes, if present, the barcode sequences B_{P}, then the barcode sequences B_{T}, then the barcode sequences B_{S}, then the UMI sequence. Thus advantageously, the position of the compartment, the time point of cultivation, the biomolecule of interest and the number of biomolecules of interest ca be determined, if said data was incorporated into the sequenceable product when performing the method.

The particular order of the analysis algorithm for identification and categorization can vary (e.g. first barcode sequences B_{T}, then the barcode sequences B_{P}, then the barcode sequences B_{S}, then the UMI sequence; or barcode sequences B_{S}, then the barcode sequences B_{T}, then the barcode sequences B_{P}, then the UMI sequence; or first barcode sequences B_{P}, then the barcode sequences B_{S}, then the barcode sequences B_{T}, then the UMI sequence; or first UMI sequence, then barcode sequences B_{S}, then the barcode sequences B_{T}, then the barcode sequences B_{P}). According to one embodiment, the analysis algorithm may be repeated multiple times. According to one embodiment, the algorithm may be repeated as many times as required until the concentration/number of the biomolecules of interest for the time points for the positions, preferably until the concentration/number of all biomolecules of interest for all time points for all positions, is determined.

Other processing steps may also be performed in frame of the present method. Such processes include but are not limited to washing, purification, extraction, separation, centrifugation, sedimentation, etc. According to one embodiment, after step c) or d) the one or more cells can be extracted in an additional step, wherein the cell can be analyzed by sequential analysis means, including NGS sequencing of the genome or selected genes.

### Further Embodiments of the Method of the First Aspect

Further embodiments of the method according to the first aspect are described in the following:

the Cell-Laden Matrix

The Matrix

According to one embodiment, the cell-laden matrix comprises one or more cells, as well as water and a network, wherein the network comprises a molecule, which is at least partially soluble in aqueous solutions. According to a preferred embodiment, the matrix comprises a hydrogel. This has the advantage to provide a water-rich environment to the cells, mimicking the conditions the cells naturally encounter. Moreover, the hydrogel can be adjusted in its properties to provide the cells a particular environment that can be freely modified. For instance, the mesh size of the hydrogel can be tuned by the concentration of the hydrogel precursor molecules and/or the molecular weight of the hydrogel precursor molecules which can be independently adjusted and combined. The tunable mesh size renders the cell-laden matrices perfectly suitable for diffusion of different adhesive ligands, bioactive compounds and functional biomolecules such as antibodies and nucleic acids for the method of the present disclosure. In particular, the tunable mesh size has the advantage to enable diffusion of one or more biomolecules of interest out of the cell-laden matrix. According to one embodiment, the one or more biomolecules of interest can diffuse out of the cell-laden hydrogel into a compartment, preferably an isolated compartment, of a cell culture device.

In one embodiment, the cell-laden matrix comprising at least one cell is a spherical particle, preferably a spherical hydrogel particle. In a preferred embodiment, the matrix comprises a hydrogel, a polymer or pre-polymer which is selected from the group comprising polyacrylamide, poly(lactic acid) (PLA), polyglycolide (PGA), copolymers of PLA and PGA (PLGA), poly(vinyl alcohol) (PVA), polyethylene glycol) (PEG), poly(ethylene oxide), poly(ethylene oxide)-co-poly(propylene oxide) block copolymers (poloxamers, meroxapols), poloxamines, polyanhydrides, polyorthoesters, poly(hydroxyl acids), polydioxanones, polycarbonates, polyaminocarbonates, poly(vinyl pyrrolidone), poly(ethyl oxazoline), carboxymethyl cellulose, hydroxyalkylated celluloses such as hydroxyethyl cellulose and methylhydroxypropyl cellulose, and natural polymers such as nucleic acids, polypeptides, polysaccharides, chitosan or carbohydrates such as polysucrose, hyaluranic acid, dextran and similar derivatives thereof, heparan sulfate, chondroitin sulfate, heparin, or alginate, and proteins including without limitation gelatin, collagen, albumin, or ovalbumin, or copolymers, or blends thereof. In particularly preferred embodiments, the monomers can be selected from polyactic acid) (PLA), poly(vinyl alcohol) (PVA), polyethylene glycol (PEG) and polyoxazoline (POx).

Particularly preferred polymers, especially for use as building-block for hydrogel formation, is a polymer of formula (P1)

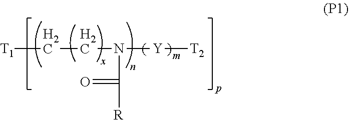

wherein
R is independently selected from a hydrogen atom, a hydrocarbon with 1-18 carbonatoms (preferably CH₃, —C₂H₅,), a C₁-C₂₅-hydrocarbon with at least one hydroxy group, a C₁-C₂₅-hydrocarbon with at least one carboxy group, (C₂-C₆)alkylthiol, (C₂-C₆)alkylamine, protected (C₂-C₆)alkylamine (preferably-(CH₂)₂₋₆—NH—CO—R (with R=tert-Butyl, perfluoroalkyl)), (C₂-C₆)alkylazide, polyethylene glycol, polylactic acid, polyglycolic acid, polyoxazoline, or wherein R is a residue R⁴
Y is a moiety containing at least one graft, comprising at least one residue R⁴,
T₁ is a terminating moiety, which may contain a residue R⁴,
T₂ is a terminating moiety, which contains a residue R⁴,
p is an integer from 1 to 10,
n is an integer greater than 1 and preferably, below 500,
m is zero or an integer of at least, preferably greater than 1, and preferably, below 500,
the sum n+m is greater than 10,
x is independently 1, 2 or 3, preferably x is independently 1 or 2, most preferably x is 1,
R⁴ independently comprise at least one functional group
   for crosslinking and/or
   for binding biologically active compounds, and
   optionally comprising a (preferably degradable) spacer moiety connecting said functional group with the binding site to the respective moiety of the structure of formula (P1),
   wherein the entirety of all m-fold and n-fold repeating units are distributed in any order within the polymer chain and wherein optionally, the polymer is a random copolymer or a block copolymer.

The entirety of the m-fold and n-fold repeating units of formula (P1) represent a polymer chain. The distribution of said repeating units within said polymer chain occurs in any possible arrangement of said repeating units within said polymer chain. If at least two distinguishable repeating units are present within said polymer chain (for example the polymer comprises units with different substituents R or m is different from zero), the polymer may be a random copolymer or a block copolymer.

In the event that m is an integer greater than 1, an alternating order of repeating units is particularly preferred, wherein one repeating unit, chosen from the portion of n-fold repeating units is directly connected to a unit, chosen from the portion of m-fold repeating units. Said alternating arrangement leads to a particularly preferred embodiment of the polymer of formula (P1) according to formula (P1-1)

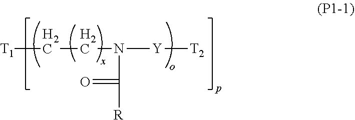

wherein o is an integer of greater than 1 and

T¹, T², x, R and Y is defined according to formula (P1).

The polymer according to formula (P1) and (P1-1) comprises an amount of p (one to ten) of said polymer chains. According to the structure of formula (P1) or (P1-1), T¹ is clearly defined as a terminating moiety, which functions dependent on the value of p either as a terminus-unit (end-cap) for p=1, or a core or branching point moiety (p=2 to 10), connecting an amount of p polymer chains. According to formula (P1), T² is clearly defined as a terminus residue (end-cap).

Preferred polymers of formula (P1) are characterized in, that R is a hydrogen atom or a C₁-C₁₈-alkyl group, (preferably a hydrogen atom, methyl, ethyl, n-propyl, iso-propyl, n-butyl, iso-butyl, sec-butyl, tert-butyl, pentyl, iso-pentyl, neopentyl, sec-pentyl, hexyl, heptyl, octyl, nonyl, decyl) and m is an integer greater than 1.

In another preferred embodiment, a polymer, especially polymer as building-block for hydrogel formation is, characterized in, that R is a hydrogen atom, a hydrocarbon with 1-18 carbonatoms (preferably CH3, —C2H5,); Y is a moiety containing at least one graft, comprising at least one degradable spacer moiety connecting at least one N-hydroxysuccinimide ester for binding biologically active compounds to the respective moiety of the structure of formula (P1); T1 is a terminating moiety, optionally comprising a peptide nucleic acid (PNA) sequence; T2 is a terminating moiety, optionally comprising a peptide nucleic acid (PNA) sequence; n is an integer greater than 1; m is an integer greater than 1; the sum n+m is greater than 10 and less than 500; and x is 1; wherein the entirety of all m-fold and n-fold repeating units are distributed in any order within the polymer chain and wherein optionally, the polymer is a random copolymer or a block copolymer.

A particularly preferred first embodiment of polymers according to formula (P1) and (P1-1) are characterized in, that
T₁ is a terminating moiety, comprising a first XNA-residue (XNA1) and optionally an EDTS-moiety,
T₂ is a terminating moiety, comprising a second XNA-residue (XNA2) and optionally an EDTS-moiety,
p equals 1 or 2, preferably equals 1,
   EDTS is an enzyme degradable target site, preferably a matrix metalloprotease (MMP) target site, for site directed degradation of the polymer,
XNA is a nucleic acid or nucleic acid analog, preferably a peptide nucleic acid (PNA) sequence.

The rest of the parameters according to formula (P1) or (P1-1) are defined as mentioned above (vide supra). The polymer of this first embodiment is a linear polymer.

A particularly preferred second embodiment of polymers according to formula (P1) and (P1-1) are characterized in, that
T₁ is a terminating moiety, comprising no residue R⁴,
T₂ is a terminating moiety, comprising a XNA-residue, optionally linked to an EDTS-moiety,
p is an integer of 3 to 10, preferably 3 to 10, preferably 3 to 8, most preferred 3 to 6,
EDTS is an enzyme degradable target site, preferably a matrix metalloprotease (MMP) target site, for site directed degradation of the polymer,
XNA is a nucleic acid or nucleic acid analog, preferably a peptide nucleic acid (PNA) sequence.

The rest of the parameters according to formula (P1) or (P1-1) are defined as mentioned above (vide supra). The polymer of this second embodiment is a star-shaped polymer.

A preferred polymer of said second embodiment is characterized in, that m is zero and no moiety Y is comprised in the polymer.

A particularly preferred third embodiment of polymers according to formula (P1) and (P1-1) are characterized in, that,
T₁ is a terminating moiety, comprising a residue R⁴ different from a XNA-residue, wherein R⁴ is optionally linked to a EDTS-moiety,
T₂ is a terminating moiety, comprising a residue R⁴ different from a XNA-residue, wherein R⁴ is optionally linked to an EDTS-moiety,
p equals 1 or 2, preferably equals 1,
EDTS is an enzyme degradable target site, preferably a matrix metalloprotease (MMP) target site, for site directed degradation of the polymer,
XNA is a nucleic acid or nucleic acid analog, preferably a peptide nucleic acid (PNA) sequence.

The rest of the parameters according to formula (P1) or (P1-1) are defined as mentioned above (vide supra). The polymer of this third embodiment is a linear polymer.

A preferred polymer of said third embodiment is characterized in, that m is zero and no moiety Y is comprised in the polymer.

A particularly preferred fourth embodiment of polymers according to formula (P1) and (P1-1) are characterized in, that
T₁ is a terminating moiety, comprising no residue R⁴,
T₂ is a terminating moiety, comprising a residue R⁴ different from a XNA-residue, wherein R⁴ is optionally linked to an EDTS-moiety,
p is an integer of 3 to 10, preferably 3 to 10, preferably 3 to 8, most preferred 3 to 6,
EDTS is an enzyme degradable target site, preferably a matrix metalloprotease (MMP) target site, for site directed degradation of the polymer,
XNA is a nucleic acid or nucleic acid analog, preferably a peptide nucleic acid (PNA) sequence.

The rest of the parameters according to formula (P1) or (P1-1) are defined as mentioned above (vide supra). The polymer of this second embodiment is a star-shaped polymer.

A preferred polymer of said fourth embodiment is characterized in, that m is zero and no moiety Y is comprised in the polymer.

A preferred polymer according to formula (P1), (P1-1) and their four preferred embodiments are characterized in, that it is a polymer which comprises an EDTS-moiety, preferably a MMP-moiety.

A preferred polymer according to formula (P1), (P1-1) and according to their four preferred embodiments is characterized in, that it comprises at least two different moieties R.

A preferred polymer according to formula (P1), (P1-1) and according to their four preferred embodiments is characterized in, that p is an integer of 3 to 10, preferably 3 to 10, preferably 3 to 8, most preferred 3 to 6.

The matrix may comprise polymers and/or precursor molecule, preferably in a predominantly crosslinked form, which have been disclosed in PCT/EP2018/074527, in particular, polymers and/or precursor molecules disclosed in claims 101 to 155, which are herein incorporated by reference.

In a preferred embodiment, the matrix comprises a hydrogel. The hydrogel may be a hydrogel as disclosed in PCT/EP2018/074527, in particular, hydrogels as disclosed in claims 1 to 51 and 72, which are herein incorporated by reference. PCT/EP2018/074527 further discloses methods for producing a hydrogel in claims 52 to 71, which are herein incorporated by reference. Furthermore, a kit for producing a hydrogel is disclosed in PCT/EP2018/074527 in claims 99 and 100, which are also herein incorporated by reference.

According to a preferred embodiment, the matrix is three-dimensional. According to one embodiment, three-dimensional may be understood as providing an environment that can be sensed spatially. For instance, a cell may sense a three-dimensional matrix around itself and not only at one side of the cell, which would be the case for planar matrixes. However, according to one embodiment of the present disclosure, three dimensional may also be understood as providing a quasi-planar environment to the cells at which cells are for instance at the border of a three dimensional matrix, wherein cells encounter a planar or curved surface at one side and a three-dimensional matrix at the other side.

According to another embodiment, further features may be provided by the three dimensional matrix, for instance by addition of further structural features such as topography, mechanical strain and shear stress into the matrix (which sometimes in the art is described as a fourth dimension but is here encompassed by the term “three-dimenional”). Furthermore, the matrix may be time- or signal-responsive, which may be understood as another dimension in the art. According to one embodiment, degradative molecules are added in order to degrade the matrix. For instance, if the matrix is crosslinked by hybridization of (complementary) molecules forming hydrogen-bridges (e.g. DNA-DNA-hybridization; PNA-PNA-hybridization; PNA-DNA-hybridization, etc.), degradative molecules may be added that interrupt the hybridization crosslinks. Thereby, the crosslinks may be released, whereupon the matrix can degrade to be capable of releasing the one or more cells.

The released cell might be further analyzed in regard to its genome and/or transcriptome by bimolecular techniques. The degradation of the hydrogel and the recovery of the cells provide the possibility to link the phenotype of the cell to the underlying genotype. According to one embodiment, methods for degrading a hydrogel matrix which have been described in PCT/EP2018/074527, in particular in claims 84 to 96 are applicable in conjunction with the present disclosure and are herein incorporated by reference. According to one embodiment, degradative molecules are secreted by encapsulated cells in order to remodel the surrounding matrix. For instance, if the matrix comprises matrix metalloproteinase target sites, cell secreted matrix metalloproteinases (MMP) degrade the cross-linked matrix. Secretion of degradable enzymes can enable cell motility and chemotaxis of the cells.

According to one embodiment, the matrix is a particle, preferably a spherical particle. The matrix is preferably a particle, having a shape selected from ellipsoidal, bead, spherical, droplet, elongated, rod, rectangular, and box. The matrix may have a regular shape, corresponding to an isometric particle or predominantly isometric particle. According to another embodiment, the matrix may have an irregular shape, corresponding to an anisometric shape.

According to one embodiment, the matrix diameter may be ≤1000 μm, such as ≤800 μm, ≤600 μm or ≤400 μm, preferably ≤200 μm. The matrix may have a diameter selected from a range of 5 μm to 1000 μm, and 10 μm to 500 μm, preferably selected from a range of 10 μm to 200 μm, 20 to 150 μm, and 50 to 100 μm. According to one embodiment, the matrix has a diameter of 80 μm. According to a preferred embodiment, the diameter of the matrix is adjusted to the size of the compartment, enabling transfer inside and out of the compartment. Moreover, the diameter of the matrix may be adjusted to the size of a microfabricated geometry for the immobilization of one or more matrices. The diameter may be considered as the longest axis of the matrix.

The One or More Cells

According to the present disclosure, the cell-laden matrix comprises at least one cell. The at least one cell may be selected from a prokaryotic and/or an eukaryotic cell. The at least one cell may be selected from the groups consisting of bacteria, archaea, plants, animals, fungi, slime moulds, protozoa, and algae. According to a preferred embodiment, the one or more cells may be selected from animal cells, preferably human cells. According to one embodiment, the at least one cell may be selected from cell culture cell lines. According to another embodiment, the one or more cells may be selected from the group consisting of stem cells, bone cells, blood cells, muscle cells, fat cells, skin cells, nerve cells, endothelial cells, sex cells, pancreatic cells, and cancer cells. According to another embodiment, the at least one cell may be derived from cells of the nervous system, the immune system, the urinary system, the respiratory system, the hepatopancreatic-biliary system, the gastrointestinal system, the skin system, the cardiovascular system, developmental biology (including stem cells), pediatrics, organoids, and model organisms. According to another embodiment, the at least one cell may be derived from one or more of blood and immune system cells, including erythrocytes, megakaryocytes, platelets, monocytes, connective tissue macrophages, epidermal Langerhans cells, osteoclast (in bone), dendritic cells, microglial cells, neutrophil granulocytes, eosinophil granulocytes, basophil granulocytes, hybridoma cells, mast cells, helper T cells, suppressor T cells, cytotoxic T cells, natural killer T cells, B cells, natural killer cells, reticulocytes, hematopoietic stem cells, and committed progenitors for the blood and immune system. According to one embodiment, the at least one cell may be derived from one or more of myoloid-derived suppressor cells type M (M-MDSC, tumor-supporting M2 macrophages, CAR-T cells, CAF (cyclophosphamide, doxorubicin, and fluorouracil)-treated cancer cells, cancer cells from residual tumors, cancer cells from relapsed tumors, cells isolated by biopsy, tumor initiating cells (TICs) and cancer stem cells, and tumor infiltrating lymphocytes (TILs).

The cell-laden matrix may comprise one cell. The cell-laden matrix may comprise more than one cell. According to another embodiment, the cell-laden matrix comprises a colony of cells. Preferably, a colony of cells can be located inside the three-dimensional matrix. According to another embodiment, the cell number changes throughout performing the method. For instance, the cell number increases over the course of cultivation, decreases over the course of cultivation or remains constant over the course of cultivation. A colony of cells may be formed by proliferation of one or more cells, wherein preferably cells proliferate inside the three-dimensional matrix. In another embodiment, the cell-laden matrix comprises at least two different types of cells that interact. In particular, the cell-laden matrix may comprise two different types of cells that interact. Alternatively, the cell-laden matrix may comprise three different types of cells that interact or four different types of cells that interact of five different types of cells that interact, or more than five different types of cells that interact.

According to another embodiment, more than one cell-laden matrix is provided. According to such an embodiment, the cell-laden matrices may be provided such, that at least two cells that interact with each other are analyzed and wherein each cell is located inside a three-dimensional matrix. Preferably, the cell-laden matrices are then located in close proximity to each other. According to an embodiment where more than one cell-laden matrix is provided, the cell-laden matrices may comprise one or more cells, wherein the cell number may be the same or different between the provided cell-laden matrices. The cell number may change throughout performing the method of the present disclosure. For instance, the cell number may increase throughout performing the method of the present disclosure, wherein the increase may be equal or different for different provided cell-laden matrices. When providing more than one cell-laden matrix, the type of cells present in per cell-laden matrix may be the same or different. For instance, immune cells may be provided in one cell-laden matrix and cancer cells in another cell-laden matrix, allowing to study the interaction of both types of cells.

According to one embodiment, migration between cells might be studied for secreted chemokines to combine information on secreted biomolecules with phenotypic function (e.g. migration of T-cell through matrix towards cancer cell (verifies that t-cell detects corresponding cytokine) and subsequent killing of cancer cell (verifies successful TCR binding as well as efficient killing of cancer cell). In addition, matrix might contain MMP (matrix metalloproteinase) sites for verification of matrix remodelling performed by efficient T-cells.

According to one embodiment, the method is not only useful for the analysis of the released molecules of single cells (or cell colonies) that are in proximity, preferably close proximity, but also for the analysis of chemoattractant-based cell-cell interaction between to different cell types (e.g. an immune cell and a cancer cell). To this end, chemo-attraction, migration and phenotypic interactions between cells positioned in two separated cell-laden matrices might be studied and linked to released biomolecules of interest (e.g. secreted chemokines). This enables identification of defined combinations of secreted biomolecules of interest with a distinct phenotypic function. For instance, the successful migration of T-cells through the matrix towards cancer cell located in a different cell matrix verifies that T-cells detect cancer cell-derived cytokine and/or chemokines and subsequent kill cancer cells by TCR recognition.

According to one embodiment, the method is not only useful for the analysis of chemoattractant-based cell-cell interactions but also for the analysis of direct cell-cell interactions. To this end, cells of the same or different type can be co-encapsulated within one cell-laden matrix (e.g. hydrogel matrix) and optionally brought into direct contact by cell-centring method disclosed in (PCT/EP2018/074526). Subsequently, the time-lapse secretion profile can be monitored as according to the present disclosure. For instance, an immune cell and a cancer cell can be co-encapsulated within one cell-laden matrix (e.g. hydrogel matrix) and the time-lapse secretion profile can subsequently be monitored as according to the present disclosure.

In one embodiment one single cell of a specific cell type is encapsulated within a matrix and subsequently positioned within a (microfabricated) compartment comprising at least one positioning mean. The analysis of released biomolecules of interest is performed as disclosed by positioning a capture matrix in close proximity to the cell-laden matrix. This has the advantage that the secretion profiles of isolated single cells can be generated which is in particular of importance for the characterization of heterogeneous cell populations and the identification of cells having unique and clinically relevant functions such as cancer stem cells. According to a preferred embodiment, methods for encapsulating at least one cell are performed as described in PCT/EP2018/074527, in claims 73 to 83, which are herein incorporated by reference.

In one embodiment two cells of different cell types are encapsulated within the same matrix and subsequently positioned within a (microfabricated) compartment. The Co-encapsulation can be performed by droplet formation using established techniques including corresponding sorting mechanism such as DEP-based sorting procedures (for instance, disclosed in PCT/EP2018/074526; Mazutis et al., 2013, Nature Protocols, 8, pages 870 to 891; Kleine-Brüggeney et al., 2019, Small, 15(5):e1804576). Subsequently a matrix (i.e. capture matrix) is positioned next to or in proximity to the cell-laden matrix within the same (microfabricated) compartment. This has the advantage that the isolated interaction of two different cell types can be analyzed. For example, one cell might secrete biomolecules that affect the neighboring cell thereby inducing certain cell responses. This is especially advantageous in the field of immuno-oncology. For example, the interaction between single cancer stem cells and single immune cells and the corresponding secreted biomolecules might give important insights on cell behavior and function. By the encapsulation of two cell types within one matrix, the distance between the two cell types can be minimized thereby increasing the chance that the cells get into direct contact. This is especially advantageous in processes where cell-cell contact is necessary for inducing a desired cell response.

In one embodiment two cells of different cell types are encapsulated within two separate matrices. Subsequently a capture matrix is positioned next to or in close proximity to the cell-laden matrices within the same (microfabricated) compartment. This has the advantage, that two cell types can be spatially separated in a controlled manner. Thus, it is possible to investigate paracrine signaling between two single cells provided in separate matrices (e.g. hydrogel matrices).

In one embodiment two or more cells of different cell types are encapsulated within separated matrices and subsequently positioned in proximity to each other within a (microfabricated) compartment. Subsequently a capture matrix is positioned next to or in proximity to the cell-laden matrices within the same (microfabricated) compartment. This has the advantage, that the interaction and signaling between multiple cell types can be studied.

In one exemplary embodiment, a cytotoxic T-cell, a macrophage and a tumor cell are encapsulated within separated matrices (preferably hydrogel matrices) and subsequently positioned in proximity of each other within a (microfabricated) compartment. Subsequently a capture matrix is positioned in proximity to the cell-laden matrices within the same (microfabricated) compartment. In frame of this embodiment, the migration of T-cells through the matrix towards cancer cell located in a different cell matrix verifies the detection of cancer cell-derived cytokine and/or chemokines by T-cells, the capability of matrix degradation and the ability to kill cancer cells by TCR recognition.

According to one embodiment, the released biomolecules of interest are released by one cell. According to another embodiment, the released biomolecules of interest are released by more than one cell. Furthermore, the number of cells that release the biomolecule of interest may change throughout performing the method according to the present disclosure. Alternatively, the number of cells may change when performing other methods or procedures. The mode of release may not be limiting according to the present disclosure. Cells may release biomolecules of interest via secretion vesicles. Cells may release biomolecules of interest via exocytosis.

The Biomolecules of Interest

According to the present disclosure, cells may release one or more biomolecules of interest. According to one embodiment, the method according to the first aspect allows to determine the profile of released biomolecules of interest. A profile of released biomolecules of interest may be understood as a time-dependent measurement of the absolute or relative amount of biomolecules of interest released and/or bound by the capture molecule. One or more biomolecules are preferably selected from the group comprising peptides, polypeptides and proteins (e.g. enzymes such as metalloproteases) and combinations thereof. Other biomolecules of interest may also be released and analyzed by the method according to the present disclosure. For instance, carbohydrates, nucleic acids, small organic molecules or lipids, glycopeptides and combinations thereof may be released (e.g. via secretion) and analyzed.

According to one embodiment, one or more types of biomolecules of interest are released. According to one embodiment, at least 1, at least 2, or at least 3 different types of biomolecules of interest are released, preferably at least 5, at least 6, at least 7, at least 8, or at least 9 different types of biomolecules of interest, more preferably 10 or more different types of biomolecules of interest are released and analyzed. According to one embodiment, one type of biomolecules is released and analyzed.

According to a preferred embodiment, the release of the one or more biomolecules of interest takes place by secretion of biomolecules. According to a particular embodiment, all biomolecules of interest that are released by secretion.

According to a preferred embodiment, the biomolecules of interest may be selected from cytokines. Moreover, one or more biomolecules of interest may be selected from the group consisting of cytokines, chemokines, interferons (INF), interleukins (IL), lymphokines, and tumor necrosis factor (TNF). According to another embodiment, the one or more biomolecules of interest are selected from the group consisting of interleukins (ILs), including IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36α, IL-36β, IL-36γ, IL-37, IL-1Ra, IL-36Ra and IL-38; interferons (INFs), including type I IFNs (such as IFN-α (further classified into 13 different subtypes such as IFN-α1, -α2, -α4, -α5, -α6, -α7, -α8, -α10, -α13, -α14, -α16, -α17 and -α21), and IFN-β, IFN-δ, IFN-ε, IFN-ζ, IFN-κ, IFN-v, IFN-τ, IFN-ω), type II IFN (such as IFN-γ) and type III IFNs (such as IFN-λ1 and IFN-λ2/3); tumor necrosis factors (TNF), such as TNF-α, TNF-β, CD40 ligand (CD40L), Fas ligand (FasL), TNF-related apoptosis inducing ligand (TRAIL), and LIGHT; chemokines, including CCL1, CCL2, CCL3, CCL4, CCL5, CCL6, CCL7, CCL8, CCL9/CCL10, CCL11, CCL12, CL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, XCL1, XCL2, CX3C, and CX3CL1; other cytokines, such perforin, granzyme, MCP-1, MCP-2, MCP-3. Rantes, IP-10. Osteopontin, MIP-1a, MIP-1b, MIP-2, MIP-3a, MIP-5, VEGF, IGF, G-CSF, GM-CSF, Eotaxin, PDGF, Leptin, and Flt-3; and/or combinations thereof.

According to one embodiment, the one or more biomolecules of interest are selected independently for each time-point. For instance, in the beginning of the experiment growth factors such as EGF and VEGF are analyzed, in the middle of the experiment, chemokines such as CCL2 and CCL5 are analyzed and in the end of the experiment interleukins such as IL-6 and IL-10 are analyzed.

The Capture Matrix

The Matrix

According to one embodiment, the capture matrix may be provided by a hydrogel, wherein the hydrogel can have one or more of the features described above in conjunction with the cell-laden matrix provided by a hydrogel. Furthermore, the capture matrix may be provided by a hydrogel, comprising a molecule, which is at least partly soluble in aqueous solutions and can be derived from one or more of the molecules described above in conjunction with the cell-laden matrix provided by a hydrogel. According to another embodiment, the capture matrix may be provided by a hydrogel comprising one or more polymers, especially a as building or for hydrogel formation, as described above.

In a preferable embodiment, the capture matrix comprises a hydrogel, which preferably has a spherical shape. In a preferred embodiment, wherein the matrix comprises a hydrogel, a polymer or pre-polymer is chosen by one skilled person in the art from at least one of the polymers, pre-polymers or precursors described above in conjunction with the cell-laden matrix. The capture matrix may comprise polymers and/or precursor molecule, which have been disclosed in PCT/EP2018/074527, in particular, polymers and/or precursor molecules disclosed in claims 101 to 155, which are herein incorporated by reference. In a preferred embodiment, the capture matrix comprises a hydrogel. The hydrogel may be a hydrogel as disclosed in PCT/EP2018/074527, in particular, hydrogels as disclosed in claims 1 to 51 and 72, which are herein incorporated by reference. PCT/EP2018/074527 further discloses methods for producing a hydrogel in claims 52 to 71, which are herein incorporated by reference. Furthermore, a kit for producing a hydrogel is disclosed in PCT/EP2018/074527 in claims 99 and 100, which are herein incorporated by reference.

In the preferred embodiment, the matrix comprises a hydrogel, wherein the hydrogel comprises a polymer or pre-polymer (preferably predominantly in a crosslinked state) which is selected from the group comprising poly(lactic acid) (PLA), polyglycolide (PGA), copolymers of PLA and PGA (PLGA), poly(vinyl alcohol) (PVA), polyethylene glycol (PEG), poly(ethylene oxide), poly(ethylene oxide)-co-poly(propylene oxide) block copolymers (poloxamers, meroxapols), poloxamines, polyanhydrides, polyorthoesters, poly(hydroxyl acids), polydioxanones, polycarbonates, polyaminocarbonates, poly(vinyl pyrrolidone), poly(ethyl oxazoline), carboxymethyl cellulose, hydroxyalkylated celluloses such as hydroxyethyl cellulose and methylhydroxypropyl cellulose, and natural polymers such as nucleic acids, polypeptides, polysaccharides or carbohydrates such as polysucrose, hyaluranic acid, dextran and similar derivatives thereof, heparan sulfate, chondroitin sulfate, heparin, or alginate, and proteins including without limitation gelatin, collagen, albumin, or ovalbumin, or copolymers, or blends thereof. In particularly preferred embodiments, the monomers can be selected from polyactic acid (PLA), poly(vinyl alcohol) (PVA), polyethylene glycol (PEG), polyoxazoline (POx) and polyacrylamide (PAM).

According to a preferred embodiment, the capture matrix can be three-dimensional. As described above, other features may also be incorporated into the capture matrix and may be applicable in view of the present disclosure.

Moreover, the capture matrix may be a particle, preferably a spherical particle. Other shapes are also applicable for the capture matrix, including those described above in conjunction with the cell-laden matrix.

The capture matrix may have a diameter selected from a range of 5 μm to 1000 μm, such as 10 μm to 500 μm, preferably selected from a range of 10 μm to 200 μm, 20 to 150 μm, and 50 to 100 μm. Further characteristics in respect to the diameter have been described above for the cell-laden matrix and may also be applicable for the capture matrix.

In one embodiment at least two capture matrices comprising different types of capture molecules (e.g. antibodies) are subsequently positioned next to or in proximity to the cell-laden matrix or matrices within the same (microfabricated) compartment.

In another embodiment the capture matrix comprises one or more types of capture molecules, in particular antibodies. The capture matrix can be selected from polymer particles (e.g. beads), magnetic particles, hydrogel spheres/matrices/beads, or resins or combinations thereof.

In another embodiment, at least one capture particle is encapsulated within a matrix, preferably a hydrogel matrix. The at least one capture particle comprises one or more types of capture molecules and can be selected by the person skilled in the art from the group consisting of polymer particles (e.g. polystyrene beads), magnetic particles, hydrogel spheres/matrices/beads, resins or capture matrices or combinations thereof. The particle diameter may be ≤200 μm, such as ≤100 μm or ≤80 μm, preferably ≤30 μm. The at least one capture particle may have a diameter selected from a range of 0.1 μm to 100 μm, and 1 μm to 50 μm, preferably selected from a range of 0.1 μm to 80 μm, 0.5 to 50 μm, and 1 to 30 μm. The capture particle is preferable smaller in diameter than the matrix encapsulating said at least one capture particle. The polymers for the hydrogel matrix can be selected from polyactic acid) (PLA), poly(vinyl alcohol) (PVA), polyethylene glycol (PEG) and polyoxazoline (POx), polyacrylamide (PMA) and agarose.

As disclosed herein, the capture matrix may be contacted with the cell-laden matrix in different orders for binding the released one or more biomolecules of interest.

The Capture Molecules

According to a preferred embodiment, the capture matrix comprises one or more types of capture molecules. In particular, the one or more types of capture molecules may be bound to the capture matrix. They are bound such, that capture molecules are capable of binding biomolecules of interest, e.g. by providing a capture matrix that allows diffusion of the biomolecule of interest into the capture matrix to the capture molecules, by binding the capture molecules such that it does not prevent the capture molecules from binding to the biomolecules of interest. In an exemplary embodiment, one or more types of capture molecules may be incorporated by reaction(s) based on:
covalent bond formation chosen from the group consisting of:
   enzymatically catalyzed reactions
      transglutaminase factor XIIIa
   not-enzymatically catalysed
      click chemistry
      photo-catalyzed
   uncatalyzed reactions
      Copper-free highly selective click chemistry
      Michael-type addition
      Diels-Alder conjugation
non-covalent bond formation:
   Hydrogen bonds formed by:
      Nucleic acids
   Hydrophobic interactions
   Van-der-Waals
   Electrostatic interactions

According to one embodiment, incorporation of one or more types of capture molecules into said capture matrix (e.g. an oxazoline-based hydrogel matrix) is implemented by peptide nucleic acids. PNA oligomers may be incorporated by amide bond formation between the NHS-ester from the hydrogel precursor molecule and the primary amine of a PNA oligomer. The capture molecule may be fused to a complementary PNA oligomer. The fusion product may then be immobilized by hydrogen bond formation between the two PNA oligomers. The capture molecule can be removed by addition of a molar excess of complementary PNA oligomers. The complementary PNA oligomers can compete with the PNA/capture fusion product. Alternatively, the one or more types of capture molecule can be fused to a complementary modified PNA oligomer. The modification may comprise a photo-cleavable linker between two PNA molecules. After hydrogen bond formation between the two PNA oligomers, the capture molecule can be easily removed by UV irradiation. In both cases the capture molecule may comprise or consist of an antibody, a small molecule, an antigen, a protein binding domain, a nucleic acid, a polysaccharide or an aptamer.

The attachment of the capture molecule, in particular a capture antibody to the capture matrix can be performed by any reaction well known by the person skilled in the art, including but not limited to ligation chemistry such as Diels-Adler reaction, Michael addition, Staudinger ligation, affinity-tags, Biotin-Avidin and native chemical ligation.

According to a preferred embodiment, the one or more types of capture molecules are attached to the capture matrix such that the capture molecules are not released throughout steps a) to c), preferably steps a) to d). In the particular embodiment, wherein the one or more types of capture molecules are attached via hybridization of PNAs, the PNA sequence hybridization is adjusted to not result in dehybridization over any of the steps a) to c) and step d) (aa), wherein before amplification (e.g. step d) (bb)) preferably, the extended barcode oligonucleotide is released from the one or more types of detection molecules and the capture matrix is removed.

According to one embodiment, the one or more types of capture molecules are selected from the group consisting of proteins, peptides, nucleic acids, carbohydrates, lipids, polymers, and small organic molecules. According to one embodiment, the one or more types of capture molecules are selected from the group consisting of antibodies, antibody fragments, hybrid antibodies, recombinant antibodies, single-domain antibodies (nanobodies), recombinant proteins comprising at least a portion of an antibody, chimeric antibodies, humanized antibodies, multiparatopic antibodies, multispecific antibodies, fusion proteins, aptamers, DNA aptamers, RNA aptamers, peptide aptamers, receptors, receptor fragments, non-antibody protein scaffolds comprising a molecular recognition moiety—including DARPins (Designed Ankyrin Repeat Proteins), Repebodies, Anticalins, Fibronectins, Affibodies, engineered Kunitz domains—Affirmer proteins, Adhiron proteins, lipocalins, lipid derivatives, phospholipids, fatty acids, triglycerides, glycerolipids, glycerophospholipids, sphingolipids, saccharolipids, polyketides, cationic lipids, cationic polymers, poly(ethylene) glycols, spermines, spermine derivatives or analogues, poly-lysines, poly-lysine derivatives or analogues, polyethyleneimines, diethylaminoethyl (DEAE)-dextrans, cholesterols, sterol moieties, cationic molecules, hydrophobic molecules and an amphiphilic molecules, preferably selected from the group consisting of antibodies, antibody fragments, and aptamers. According to a preferred embodiment, the one or more types of capture molecules are antibodies or antigen binding fragments thereof. According to one embodiment, an antibody is used as a type of capture molecule, wherein the antibody specifically binds a first biomolecule of interest at a first epitope. This concept may also be used for further biomolecules of interest.

According to the present disclosure, the capture matrix can comprise one or more types of capture molecules, wherein each type of capture molecule is capable of specifically binding a biomolecule of interest. According to one embodiment, the capture matrix comprises at least 2, at least 3, at least 4, at least 5, at least 6, at least 7, at least 8, at least 9, or at least 10 different types of capture molecules, wherein each capture molecule is capable of specifically binding a biomolecule of interest and wherein each type of capture molecule is capable of binding to a different type of biomolecule. It is within the scope of the present disclosure that a type of capture molecule is provided by different kinds of capture molecules (e.g. antibody and antibody fragment) which bind to the same type of biomolecule of interest (e.g. at the same or a different epitope). According to a preferred embodiment, one type of capture molecules comprises multiple identical capture molecules. Preferably multiple capture molecules of each type are bound to the capture matrix. This ensures efficient capture of released biomolecules of interest.

In one embodiment, the capture matrix comprising capture molecules (e.g. immobilized detection antibodies) can be provided (e.g. by positioning positioned the capture matrix in the compartment) in a delayed manner to improve the natural phenotype of cells such as autocrine and paracrine signaling. The release of various cytokines can dependent on each other (e.g. IL-2 and IL-5). If IL-2 is reduced, for instance, due to capture by binding to the capture molecules, no IL-5 is secreted resulting in false negative results. Such an effect can be avoided by cultivation of cells without presence of capture molecules for a prolonged period and addition of the capture matrix with capture molecules afterwards. To prevent loss of biomolecules of interest during the positioning of the capture matrix a biphasic compartment generation (for example as disclosed in PCT/EP2018/074526) can be used. Thus, the cell-laden matrix located within an aqueous phase is first positioned within a compartment of the cell culture device (preferably a microfabricated cell culture device). Afterwards, the aqueous phase is exchanged by an oil phase (such as fluorinated oil (e.g. HFE-7500)) and the cell-laden matrix is incubated for a defined period. Then, a capture matrix located within an aqueous droplet is delivered via the oil phase to the position of the cell-laden matrix. This has the advantage that the secreted molecules are not washed away as soon as the capture matrix is positioned next to the cell-laden matrix.

The Detection Molecules

According to one embodiment, the one or more types of detection molecules are capable of binding to the one or more biomolecules of interest secreted by the at least one cell of the cell-laden matrix. According to a preferred embodiment, the one or more types of detection molecules bind to a different region of the one or more biomolecules of interest than the one or more types of capture molecules. Hence, according to a preferred embodiment of the present disclosure, the capture molecule and detection molecule comprise molecular recognition moieties, which recognize different regions of a biomolecule of interest. The advantage of such an embodiment is that the two molecules do not compete for binding a biomolecule of interest but are both capable of binding to the same biomolecule.

The detection molecule can be a fusion molecule between a binding molecule and the barcode label (which may be a nucleic acid oligomer) comprising a barcode sequence B_{S} indicating the specificity of the detection molecule. According to one embodiment, the detection molecule comprises a small molecule, an antigen, an antibody, a protein binding domain, a nucleic acid, a polysaccharide or an aptamer (suitable options are also described in conjunction with the capture molecules and it is referred to the respective disclosure). Preferably, the detection molecule comprises an antibody or antibody fragment that binds the biomolecule of interest. The binding partner (biomolecule of interest) of the capture molecule can be analyzed directly within the hydrogel matrices or after separation. This procedure enables a time-lapse profiling of biomolecules of interest of single cells or of small colonies in a multiplexed fashion.

According to a preferred embodiment, one or more types of detection molecules are added in step c). It may also be preferred to provide the same number of types of detection molecules and types of capture molecules. Such an advantageous embodiment enables to first capture one or more biomolecules of interest, which may be of a different type, and sequentially bind the captured biomolecules with a corresponding type of detection molecule. Hence, biomolecules of interest are first captured, followed by binding of a detection molecule that comprises a barcode label to either directly or sequentially label the biomolecule of interest such that it can be sequentially detected and/or analysed. Thus, each biomolecule of interest to be detected has a corresponding type of capture molecule and type of detection molecule. Thus, for a cytokine 1 as first biomolecule of interest, there is a matching type of capture molecule that specifically binds cytokine 1 as well as a matching type of detection molecule that specifically binds cytokine 1. For a cytokine 2 as second biomolecule of interest, there is a matching type of capture molecule that specifically binds cytokine 2 as well as a matching type of detection molecule that specifically binds cytokine 2. The same concept can be used for further biomolecules of interest. The preferred number of types of detection molecules has been described above in conjunction with the number of types of capture molecules and it is here referred to these numbers, which also apply for the number of types of detection molecules.

According to another embodiment, a different number of types of detection molecules and capture molecules may be applied. Such an embodiment may be found useful in case a type of capture molecules is capable of binding more than one type of biomolecule of interest (e.g. of the biomolecules of interest have a similar or equal recognition moiety). Afterwards, for detection, it may be useful to differentiate between the biomolecules of interest and thus provide a greater number of types of detection molecules than the number of types of capture molecules. Vice versa, it may also be useful to provide a greater number of types of capture molecules than detection molecules (e.g. in case the detection molecules bind a number of similar biomolecules of interest, where a differentiation between the different types of biomolecules (e.g. subspecies of biomolecule types) is not required and/or desired).

In one embodiment the attachment between the barcode label and the respective detection molecule can be a linker. Preferably, the linker is a cleavable spacer, include but not limited to photocleavable linker, hydrolyzable linkers, redox cleavable linkers, phosphate-based cleavable linkers, acid cleavable linkers, ester-based cleavable linkers, peptide-based cleavable linkers, photocleavable linkers, and any combinations thereof. Preferably, the linker can be cleaved by light.

In one embodiment, the barcode label of the detection molecule is labeled with a fluorescent marker such as fluorescent organic molecules (e.g. FITC), Pyrene, Atto or quantum dots. The amount of biomolecules of interest that are bound to the one or more type of capture molecules might be determined by measuring the fluorescence intensity of the fluorescently labeled detection molecules (e.g. antibodies). As the fluorescence intensity of the detection molecules is proportional to the amount of detection molecules located within the capture matrix which is in turn proportional to the bound biomolecules of interest, an indirect quantification of bound biomolecules of interest is possible. The fluorescence intensity of the capture matrix might be analyzed using an optical set-up such as an epifluorescence microscope, a confocal laser scanning microscope, a high content screening system or a super-resolution microscope, fluorescence-activated cell scanning (FACS) or any other optical setup. The fluorescence signal of individual cytokines such as IL-6 might be used as a trigger for the highly multiplexed cytokine quantification by oligonucleotide extension reactions. Thus the fluorescence signal of a few cytokines can be used as a basis for decision whether the whole cytokine profile should be quantified or not. The detection molecules might be labeled with different fluorescent molecules or quantum dots having different excitation and emission wavelength which allows the read-out of a detection molecule with a defined specificity by using a corresponding optical-setup (multiplexing). The use of fluorescently labeled detection molecules is well known in the art, e.g. from other techniques such as fluorescent-activated cell sorting (FACS).

The Device Comprising the Cell-Laden Matrix and the Capture Matrix

According to a preferred embodiment, the cell-laden matrix and the capture matrix are provided in proximity to each other within an isolated compartment of a device. According to another embodiment, a plurality of cell-laden matrices and capture matrices are provided in a cell culture device comprising a plurality of compartments, in particular an isolated compartment or compartments that can be isolated from each other, wherein a cell-laden matrix and a capture matrix are provided within a compartment, in particular an isolated compartment, of the cell culture device. According to a preferred embodiment, a device which can be utilized for performing the method of the present disclosure corresponds to a device disclosed in PCT/EP2018/074526 in claims 40 to 73 and these are herein incorporated by reference. Other devices are also described elsewhere herein and in the examples.

According to a preferred embodiment, the method is performed utilizing a cell culture device, preferably a microfabricated cell culture device. The device may comprise a compartment for accommodating one or more cell-laden matrices. Preferably, the device comprises an array of compartments for accommodating cell-laden matrices. Moreover, the device may comprise a fluid reservoir and fluid channels for supplying fluid to the compartment. The device preferably further comprises means to switch the compartment between a closed state and an open state, wherein the closed state corresponds to a state at which fluid that is present in the compartment is in no contact with fluid not present in the compartment and wherein the open state corresponds to a state at which fluid that is present in the compartment is in contact with fluid not present in the compartment. The closed state of the compartment may correspond to an isolated compartment, wherein an at least partially closed system may be provided.

The device may comprise one or more microfabricated valves, wherein preferably the one or more microfabricated valves are capable of switching the compartment between an open and closed state. According to a preferred embodiment, the microfabricated valve comprises a first channel, a second channel, a connection channel connecting the first channel and the second channel, a valve portion arranged within the connection channel, wherein the valve portion is adapted to selectively open and close the connection channel. Moreover, the microfabricated valve may comprise at least three layers, wherein a first channel is located within a first layer; a second channel is located within a third layer; a valve portion is located within a second layer; the second layer is arranged between the first and the third layer.

The device may comprise microfabricated geometries and means for handling and processing of particles, in particular hydrogel matrices. Said handling and processing includes for example geometries and means for the positioning of particles at a pre-defined location, the storage of said particles at the position for a pre-defined period, the controlled retrieval of positioned particles and the transfer of retrieved particles to another pre-defined location.

Furthermore, the device may comprise a microfabricated valve, wherein a first channel comprises a positioning mean suitable for positioning one or more cell-laden matrices and/or capture matrices being contained in a fluid which flows through the first channel, wherein the positioning mean is arranged within the first channel in such a way that a fluid flow can be reduced by the positioning means, in particular, the positioning means narrows the cross section of the channel. According to one embodiment, the device comprises a second channel comprising a positioning mean suitable for positioning one or more cell-laden matrices and/or capture matrices being contained in a fluid which flows through the second channel, wherein the positioning means is arranged within the second channel in such a way that a fluid flow can be reduced by the positioning means, in particular, the positioning means narrows the cross section of the channel. According to a preferred embodiment, the device comprises one or more compartments for accommodating one or more cell-laden matrices and/or capture matrices, wherein a positioning mean is present suitable for positioning the one or more cell-laden matrices and/or capture matrices inside the compartment.

Positioning and Removal of Matrices

The first advantage is that matrices with defined characteristics (such as size, composition (e.g. immobilization of compounds or cells)) can be positioned on said array of the cell culture device in a programmable manner. For example, if said array has n×m microfabricated individualizable compartment (n representing the number of rows and m representing the number of columns), a defined number of particles, in particular spherical hydrogel matrices with defined characteristics can be positioned in each of the n×m microfabricated compartments. Thus, one microfabricated compartment might contain one or more matrices that might contain no, single or multiple cells of the same or of different type or that might contain capture molecules. For example, a first matrix that contains one single cell of cell type 1 might be positioned next to a matrix that contains one or more types of capture molecules in one microfabricated compartment.

A second advantage in comparison to the prior art is that said immobilized matrices can be removed in a defined way from said array at any time-point and from any position and said removed matrices can subsequently be transferred into another format such as a well plate or similar format. In addition, removal of said matrices does not affect matrix integrity (e.g. hydrogel integrity) and thus results in a higher cell viability as well as in a maintenance of any information (such as bound molecules) that might be associated with the matrices. For example, if a first matrix is located within a microfabricated compartment at position (n, m) and a second matrix is located within close proximity to the first matrix or is in direct contact with the first matrix, the second matrix might be removed first while the first matrix stays within the microfabricated compartment. Afterwards, the first matrix might be removed in a second step. This can also be done for more than two matrices.

A further advantage of the present disclosure is that matrices located within different microfabricated compartments can be removed simultaneously. For example, a first matrix located within a microfabricated compartment (n1, m1) might be removed at the same time at which a second matrix located within a microfabricated compartment (n2, m2) is removed. This can also be done for more than two matrices located at more than two different positions. Thus, the advantage is a significant reduction of time needed for removing said matrices and transferring them into another format suitable for a corresponding downstream analysis.

Controllable Fluid Perfusion

A further advantage of the cell culture device is that microfabricated compartments can be individually perfused with a fluid. For example, cells located in matrices (i.e. cell-laden matrices) positioned in an array of said cell culture device can be continuously or stepwise perfused with fresh cultivation medium resulting in a removal of cellular waste products and supply with fresh nutrients. Thus, cells can be cultivated within n×m microfabricated compartments for an extended period as new nutrients can be supplied continuously whereas all microfabricated compartments might have the same culture conditions.

In addition, the individual perfusion of said microfabricated compartment offers the advantage, that matrices located within said microfabricated can be individually perfused with fluids necessary for the processing of matrices such as washing, delivery of molecules (e.g. binding molecules, detection molecules, oligonucleotide, etc.), immobilization of other molecules within said matrices (e.g. binding molecules, capture molecules, oligonucleotides).

A further advantage of the cell culture device is that microfabricated compartments can be sequentially perfused with fluids of different compositions of the same or of different type. For example, microfabricated compartments with immobilized matrices inter alia containing cells might be first perfused with a solution containing a first molecule against specific biomolecules. Afterwards, said cell culture device might be perfused with a solution that removes the first molecule. Afterwards, said array might be perfused with a second molecule. This process might be repeated many times resulting in the addition of multiple molecules to cells located within n×m microfabricated compartments.

The Microfabricated Valve

According to one embodiment, the present disclosure relates to microfabricated structures and methods for the control of fluid flows within said cell culture device using a microfabricated (elastomer) valve. According to a preferred embodiment, the cell culture device comprises a microfabricated valve as disclosed in PCT/EP2018/074526 in claims 1 to 39, herein incorporated by reference. One of the main advantages of said microfabricated valve is that it can be used for performing and improving the most critical and important processes used in microfluidic devices as well as in the field of microdroplet microfluidics and in particular, for the generation of the disclosed array. In particular, these processes include control of fluid flows, fluid pumping and fluid mixing in microfluidic devices as well as the formation of droplets, formation of encapsulation, in particular single-cell encapsulations, co-encapsulation, droplet mixing, the formation of (hydrogel) matrices and droplet de-mulsification in terms of microdroplet-based microfluidics. The main advantage of said microfabricated elastomer valve is the low actuation pressure (<100 mbar) that is needed for its actuation as well as the nominal diameter that is suitable for the transport of larger matrices. Another advantage of the elastomer valve is that it can be fabricated in a cost-effective and simple manner using standard multilayer lithography methods. In a first embodiment said microfabricated structure for flow control comprises a first microfabricated layer with recesses comprising a first microfabricated channel which is defined as “first flow channel” and a second microfabricated layer that has a recess which connects the first microfabricated channel with the space above the second microfabricated channel. This recess is defined as “connection channel”. The connection channel is separated by a second recess of the second microfabricated layer by a thin elastomeric membrane with a thickness between 1 μm and 80 μm. The first flow channel might contain a first fluid and the space above the second microfabricated layer might contain a second fluid of the same or of different type. The recess within the second microfabricated layer that is separated by an elastomeric membrane from the connection channel is here defined as “actuation channel”.

Channels

The term “channel” requires at least any cavity which is adapted to accommodate a fluid. In an embodiment the channel may constitute a part of a conduct for conducting a stream of fluid. A channel may be a formed by a fluid conduct; a channel may be formed by a reservoir. Such a reservoir may be closed or may be open with a connection to the atmosphere. In an embodiment the channel may be a reservoir. For example, this reservoir may be closed except for the opening which connects it to another channel. Alternatively the reservoir may be open, for instance it may have an open upper end. In a one embodiment the second channel is a reservoir, in particular an open reservoir.

The Valve Action

Opening and closing of valve. In one embodiment, the actuation channel contains a fluid such as air or fluorinated oil (e.g. HFE-7500 (Novec)). Upon increasing the pressure in said actuation channel, a pressure difference between the connection channel and the actuation channel is generated. Thus, an actuation force is acting on the elastomeric membrane separating the connection channel and the actuation channel. This actuation force results in a bending of the membrane and a closing of the connection channel thereby separating the first flow channel from the space above the second microfabricated layer. After removing said pressure, the connection channel opens again due to the elastomeric characteristics of the used membrane. In a particular embodiment, the deflection distance of the membrane might be in the range of 1 μm to 100 μm. In another embodiment, the connection channel is not fully closed and thus the hydrodynamic resistance of the connection channel can be controlled in a defined manner by changing the applied pressure and thus the actuation force acting on the membrane. In one embodiment, the pressure might be varied between 0 mbar and 4000 mbar (absolute pressure) in steps of 1 mbar to adjust the hydrodynamic resistance of the connection channel. In particular embodiments the actuation force might be applied by using fluids (hereinafter also referred to as control fluid or actuating fluid) of the following type:
Gases such as air, nitrogen and argon
Liquids such as water, silicon oils, fluorinated oils and other oils
Solutions containing salts and/or polymers such as polyethylene glycol or glycerol
Ferromagnetic fluids
Hydrogels that are capable of swelling and shrinking upon application of a stimulus.

For example said stimulus might be one of the following types: temperature, ionic strength, electric field strength, magnetic field strength, pH value

In addition to applying an actuation force via a pressure-based actuation system, valve actuation might be performed by other actuation systems that might be of the following types: electrostatic, magnetic, electrolytic or electrokinetic.

Valves can be actuated by injecting gases (e.g., air, nitrogen, and argon), liquids (e.g., water, silicon oils and other oils), solutions containing salts and/or polymers (including but not limited to polyethylene glycol, glycerol and carbohydrates) and the like into the control channel, a process preferred to as “pressurizing” the control channel. In addition to elastomeric valves actuated by pressure-based actuation systems, monolithic valves with an elastomeric component and electrostatic, magnetic, electrolytic and electrokinetic actuation systems may be used. See, e.g., US 20020109114; US 20020127736, and U.S. Pat. No. 6,767,706.

In particular embodiments valves (including valves with dimensions as described above) do not completely block the flow channel lumen with the membrane is fully actuated by a control channel pressure of 30, 32, 34, 35, 38 or 40 psi.

Fluid injection. In another advantageous embodiment, the space above the second microfabricated layer is composed of a recess within a third microfabricated layer that is defined as “second flow channel”. The second flow channel might contain a fluid of type 2 and the first flow channel might contain a fluid of type 1 with fluid of type 2 and fluid of type 1 being miscible. A defined amount of the fluid of type 2 might be injected into the fluid of type 1 by applying a hydrodynamic pressure within the second flow channel that is larger than the hydrodynamic pressure in the first flow layer and by opening said elastomer valve for a defined time (e.g. 0.1 ms to 500 ms). The main advantage of using said microfabricated elastomer valve for injection of a fluid is the short opening and closing time that is needed due to the low actuation pressure resulting in a very fast valve operation. The opening time may be for example be 1, 2, 3, 4, 5 ms, s. or min. Methods for injecting a fluid have been described in PCT/EP2018/074526 in conjunction with claims 74 to 77 and 95 to 98 relating to methods for creating droplet, which are herein incorporated by reference. A droplet may comprise hydrogel particles, a hydrogel matrix, hydrogel beads, hardened and/or gelled and/or polymerized hydrogels or any other accumulated particles in particular are bonded to each other in a chemical or physical way (e.g. by surface tension), that keeps the particles together and delimits the accumulated particles from the environment, in particular a fluid surrounding the particles. According to a preferred embodiment, a droplet when injected comprises or consists of a liquid. In case the droplet comprises a liquid, the droplet predominantly consists of a liquid but further components can be present, for instance, as in a suspension (e.g. a micro- or nanoparticle suspension). After droplet injection, preferably one or more compound present in the predominantly liquid droplet react to form a matrix. Compounds which may be applied to form a matrix have been described above in conjunction with the cell-laden matrix and the capture matrix and it is here referred to those compounds. For instance, one or more of the polymers, pre-polymers, buildings blocks, precursor, monomers, etc. may be applied to generate a matrix after droplet formation. In one particular example, a precursor is dissolved and then injected to form a droplet. Over the course of transporting the droplet, the precursor may react, e.g. by polymerizing, to form a matrix. Various modes of matrix formation may be applicable in scope of the present disclose and have been described for instance in PCT/EP2018/074527.

Parallel actuation. In another advantageous embodiment, multiple microfabricated elastomeric valves might be actuated simultaneously which increases the process speed by parallelization. To this end, multiple microfabricated valves are located within the same actuation channel. If an actuation force is applied in said actuation channel, all microfabricated valves are closed at the same time. Each microfabricated valve might have a first and a second flow channel as described above which are separated from the first and second flow channels of the other microfabricated valves. Thus, different fluids located in the second flow channels might be injected simultaneously into different fluids located in the first flow channel. In another embodiment, all microfabricated valves are connected to the same second flow channel.

Layers

In an especially preferred embodiment, the microfabricated valve comprises at least three layers, wherein the first channel is located within a first layer, the second channel is located within a third layer, the valve portion is located within a second layer and the second layer is arranged between the first and the third layer.

The use of three layers enables manufacturing of a vast number of different microfabricated valves. This increases the design variety and allows designing microfabricated valves according to different process requirements, like mixing of different fluids.

Moreover, this embodiment provides a vast number of possible valve designs and allows configuring the microfabricated valve according to the desired application.

Microfabricated Compartments

Microfabricated compartment for matrix immobilization and removal. In another aspect, the present disclosure relates to microfabricated structures and methods for the controlled positioning and sequential removal of matrices within microfabricated compartments. According to a preferred embodiment, methods as disclosed in PCT/EP2018/074526 in claims 78 to 98 can be utilized in scope of the present disclosure. Hence, the said disclosure, in particular claims 78 to 98 are herein incorporated by reference.

In a first advantageous embodiment, microfabricated compartments located within said array might have at least one inlet and one outlet. A first microfabricated compartment at position (1,1) might be connected to a second microfabricated compartment (2,1). To this end, the outlet of microfabricated compartment (1,1) acts as an inlet for microfabricated compartment (2,1). The microfabricated compartment at position (2,1) might be connected to a third microfabricated compartment (3,1). Thus, all microfabricated compartments from one column n might be connected so that microfabricated compartment (n−1,1) is connected to microfabricated compartment (n,1). In addition, a microfabricated compartment positioned at (n,1) might be connected to a microfabricated compartment (1,2) which might be connected to a microfabricated compartment positioned at (2,2). This might be repeated so that all microfabricated compartments can be perfused simultaneously with the same fluid. The inlet of microfabricated compartment (1,1) might be connected to a reservoir for supply with different fluids. The outlet of the microfabricated compartment (n,m) might be connected to a collection reservoir. Thus, all connected microfabricated compartments might be perfused with the same fluid. For example, said perfusion fluid might be an aqueous phase containing nutrients or a suspension containing one or more matrices. The inlets and outlets of said microfabricated compartments might be closed by using an elastomer valve as described within the present disclosure. Microfabricated compartments might be first loaded with a fluid and then isolated from each other by closing said valves. Thus, a fluid volume located within microfabricated compartment (1,1) cannot be mixed with a fluid volume located within another microfabricated compartment (n,m). This has the advantage that the cell-cell communication between cells located within different microfabricated compartments might be prevented which is of importance as any secreted molecules from cells located within a first microfabricated compartment might influence the cell response of cells located within a second microfabricated compartment.

Positioning on Chip

Sequential positioning. In another embodiment, said connected microfabricated compartments might be perfused with a solution containing one or more particles in particular hydrogel matrices. Said microfabricated compartments might contain a microfabricated geometry for the positioning of matrices in particular for hydrodynamic trapping of matrices. If a first microfabricated compartment does not contain any matrices, a first matrix entering said microfabricated compartment will likely be positioned within a microfabricated trapping geometry. The positioning of said first matrix might change the hydrodynamic resistance of the microfabricated compartment so that a second matrix that enters said microfabricated compartment moves into a bypass channel and afterwards enters a second microfabricated compartment. Said second matrix might be immobilized within the second microfabricated compartment. A third matrix might then bypass the first and the second microfabricated compartment, entering the third microfabricated compartment. Thus, matrices might be positioned in connected microfabricated compartments in a sequential manner—a first incoming matrix might be positioned within a first microfabricated compartment, a second incoming matrix might be positioned within a second microfabricated compartment and so on.

Defined positioning of matrices with different compositions. In another embodiment, matrices located within microfabricated compartments of said array might have different compositions. For example, a first matrix of type 1 might be generated by the on-demand formation and fusion of several droplets into one larger droplet and subsequent positioning of said droplet for cell/particle centering, hydrogel formation and demulsification as is described in the prior art. The matrix might be located within a microfluidic channel that is connected to a first microfabricated compartment. Thus, a pressure might be applied so that the matrix enters said microfabricated compartment and said matrix of type 1 might be positioned in said first microfabricated compartment. Said process might be repeated for the generation of a matrix of type 2 which is subsequently positioned within a second microfabricated compartment located next to said first microfabricated compartment. This process composed of matrix generation and immobilization might be repeated until all microfabricated compartments contain one matrix.

Positioning of two matrices within one microfabricated compartment. In another embodiment, said microfabricated compartments might have a microfabricated geometry for the positioning of two matrices of the same or of different type either in contact or in close proximity. To this end, a first microfabricated compartment might have a trapping geometry as well as a bypass channel. If a first matrix enters said first microfabricated compartment, the matrix moves into the trapping geometry as the main volume flow goes through said trapping geometry. A second matrix entering said first microfabricated geometry might enter the same trapping geometry as the hydrodynamic resistance of the bypass channel is larger than the hydrodynamic resistance of the trapping geometry containing one matrix. After trapping of two matrices the hydrodynamic resistance of said microfabricated trapping geometry increases and a third matrix moves into the bypass channel and afterwards to a second microfabricated compartment.

Positioning of three matrices within one microfabricated compartment. In another embodiment, said microfabricated compartments might have a microfabricated geometry for the positioning of three matrices of the same or of different type either in contact or in close proximity. To this end, a first microfabricated compartment might have a trapping geometry as well as a bypass channel. If a first matrix enters said first microfabricated compartment, the matrix moves into the trapping geometry as the main volume flow goes through said trapping geometry. A second matrix entering said first microfabricated geometry might enter the same trapping geometry as the hydrodynamic resistance of the bypass channel is larger than the hydrodynamic resistance of the trapping geometry containing one matrix. This is also true for a third matrix entering said first microfabricated compartment. After trapping of three matrices the hydrodynamic resistance of said microfabricated trapping geometry increases and a fourth matrix moves into the bypass channel and afterwards to a second microfabricated compartment.

Further configurations, wherein more than three matrices may be positioned or two or more matrices may be positioned within one compartment are also applicable in view of the present disclosure.

Reverse Flow Cherry Picking (RFCP)

According to one embodiment, the cell-laden matrix is preferably located inside a three-dimensional matrix and is releasably fixed by a positioning mean inside a compartment. Moreover, the cell-laden matrix and/or the capture matrix can be releasably fixed by a positioning mean inside a compartment, in particular within the same compartment. According to one embodiment, the cell-laden matrix and capture matrix are fixed by a positioning mean inside a compartment, wherein the positioning mean has one or more of the following characteristics:
it is capable of fixing the cell-laden matrix and the capture matrix next to each other, wherein optionally, the cell-laden matrix and the capture matrix may be in direct contact with each other or positioned with a distance between both matrices of less than 100 μm, 50 μm, 30 μm, 10 μm, 5 μm, or 1 μm; The cell-laden matrix and the capture matrix may contact each other at a single or multiple points, in particular they may share the same contact surface.
it is capable of fixing at least one cell-laden matrix and the capture matrix next to each other;
it is capable of fixing more than one cell-laden matrix, wherein the cell-laden matrices comprise either a single cell and/or a colony located inside a three-dimensional matrix, and the capture matrix next to the more than one cell-laden matrix; and/or
it is capable of fixing two cell-laden matrices, which are each located inside a three-dimensional matrix, and the capture matrix next to each other.
it is capable of fixing at least one cell-laden matrix and at least one capture matrix to each other.

According to one embodiment, the cell-laden matrix and the capture matrix can be fixed by a positioning mean inside a compartment, wherein the compartment accommodating the cell-laden matrix is different from the compartment accommodating the capture matrix and wherein both compartments can be switched to be either in fluid contact with each other or to be in no fluid contact with each other.

According to one embodiment, the compartment can have a valve arrangement adapted to provide a fluid passing through a positioning mean wherein the valve arrangement is adapted to selectively change the direction of fluid passing the location, in particular wherein a fluid is directed such urging the cell-laden matrix and/or the capture matrix into the positioning mean and a fluid in the second direction urging the cell-laden matrix and/or the capture matrix out of the positioning mean, and in particular fluid in the second direction delivering the cell-laden matrix and/or the capture matrix in direction of an exit section. Thereby, preferably, the cell-laden matrix and/or the capture matrix may be transported into a fixed position of the compartment, as well as transported out of a fixed position towards and exit section. The cell-laden matrix and/or the capture matrix may further be transported to other compartments to store and process the cell-laden matrix and/or the capture matrix. According to a preferred embodiment, the capture matrix can be introduced into and removed from a compartment in order to capture secreted biomolecules of interest for a tailorable amount of time. After the tailored amount of time has passed, the capture matrix can be removed from the compartment and another capture matrix can be added.

According to one embodiment, detection matrices can be obtained from the (isolated) compartments and be transferred to a separate device comprising a plurality of compartments, wherein each detection matrix is transferred to an isolated compartment of the device.

In a preferred embodiment, said device is a 96-well plate, a 384-well plate, a 1536-well plate.

Further Embodiments of RFCP

Removal of matrices from position (n,m). In one advantageous embodiment, matrices might be located within a microfabricated chamber at position (n, m) within said n×m array that enables the spatial immobilization of matrices as well as the transfer of said matrices into another format such as a 96-well plate at a desired time-point.

To this end, said microfabricated compartment might comprise a microfabricated geometry for the immobilization of matrices. In addition, said microfabricated compartment might contain at least two inlets and two outlets—a first inlet and a first outlet as well as a second inlet and a second outlet. The first inlet and the first outlet might be closed by using a first microfabricated valve as described previously. In addition, the second inlet and the second outlet might be closed using a second microfabricated valve as described previously as well.

For the immobilization of matrices, said microfabricated compartment is perfused with fluid containing single or multiple matrices from the first inlet to the first outlet while the second inlet and the second outlet are closed. Afterwards, the first inlet and the first outlet might be closed and the microfabricated compartment might be perfused with a perfusion fluid from the second inlet to the second outlet.

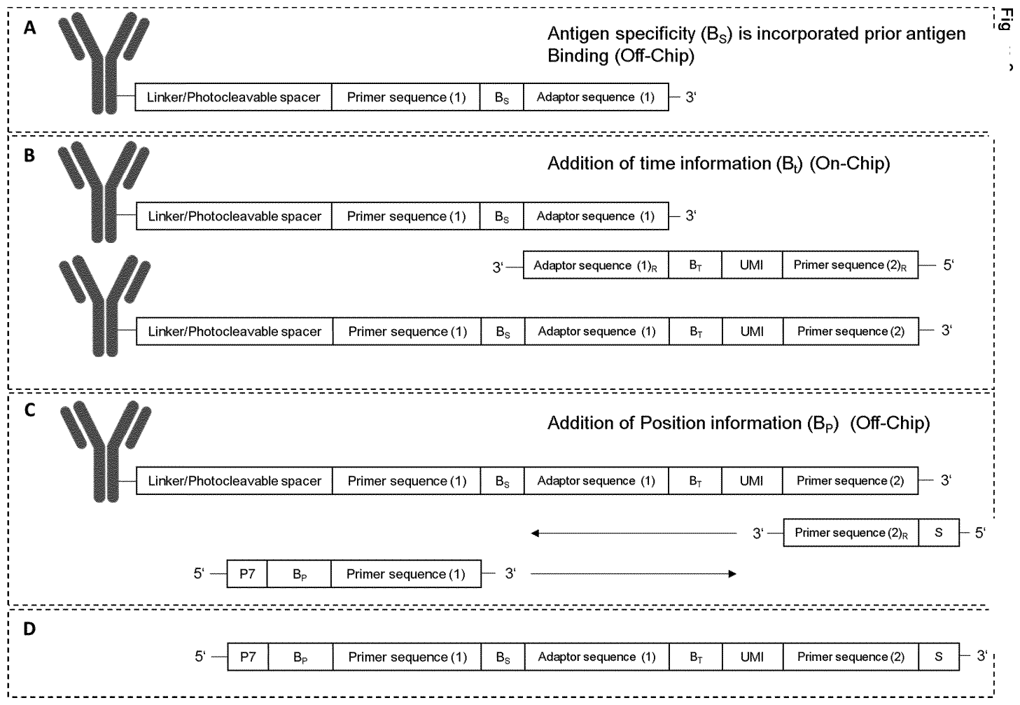

Embodiments of said trapping geometry will be described in a following section of this disclosure. Said trapping geometry is connected to at least four microfluidic channels with defined hydrodynamic resistances, a first and a second microfluidic channel having a hydrodynamic resistance R₂ and R₃, respectively and a third and a fourth microfluidic channel with the hydrodynamic resistances R₄ and R₁, respectively (FIG. 11). The hydrodynamic resistances of the first and the second microfluidic channel (R₂ and R₃) might be increased by using microfabricated valves such as described previously (elastomer valve) with a first microfluidic valve v₁ (Vm2) for controlling the hydrodynamic resistance R₂ and a second microfluidic valve v₂ (Vn2) for controlling the hydrodynamic resistance R₃. The microfabricated structure comprising a microfabricated geometry for the immobilization of matrices might have the resistance R₀. The first microfluidic channel might be connected on one side with the fourth microfluidic channel as well as with the microfabricated geometry for matrix immobilization (defined here as node N₀₁₂) and on the other side with the third microfluidic channel (defined here as node N₂₄). In addition, the third microfluidic channel might be connected on the other side to the microfabricated geometry for matrix immobilization as well as to the second microfluidic channel (defined here as node NO₄). The second microfluidic channel might be connected on the other side to the fourth microfluidic channel (defined here as node N₁₃) which might be connected to the first microfluidic channel and the microfabricated geometry for matrix immobilization (node N₀₁₂) (FIG. 11). The hydrodynamic pressure p1 at the intersection of the first microfluidic channel and the third microfluidic channel (node N₂₄) might be higher than the hydrodynamic pressure p₂ at the intersection of the second and the fourth microfluidic channel (node N₁₃). The described hydrodynamic resistances, pressures and connections are analogous to an unbalanced Wheatstone bridge known from electronic circuits. A microfabricated geometry having said resistances and characteristics is here considered as a “reverse flow cherry picking (RFCP)” geometry. A matrix might be immobilized within said microfabricated geometry for matrix immobilization. A volume flow of a fluid from node N₀₁₂ to node N₀₃₄ might perfuse the microfabricated geometry for immobilization and an immobilized matrix might stay within its position. A volume flow of a fluid from node N₀₃₄ to node N₀₁₂ might result in a removal of said matrix from its position as the volume flow is reversed (this condition is defined here as “reverse flow” condition). An immobilized matrix might require a reverse flow with a critical flow rate of Q_{crit} to be removed. Thus, a reverse flow with a flow rate Qᵣₑᵥₑᵣₛₑ below Q_{crit} (Qᵣₑᵥₑᵣₛₑ<Q_{crit}) might not result in a removal of said matrix. In contrast, a reverse flow with a flow rate Qᵣₑᵥₑᵣₛₑ larger or equal than Q_{crit} might result in a removal of said immobilized matrix from its immobilization position. Depending on the actuation of the microfabricated valves v₁ (Vm2) and v₂ (Vn2) four different conditions might be distinguished:
1. Both valves are not actuated: In terms of this condition, the hydrodynamic resistances R₂ and R₃ are smaller than the hydrodynamic resistances R₄ and R₁. The microfabricated geometry for the immobilization of matrices is mainly perfused from node N₀₁₂ to node N₀₃₄. Thus, an immobilized matrix stays within its position as the volume flow is not reversed.
2. Only valve v₁ (Vm2) is actuated while v₂ (Vn2) is not actuated: In terms of this condition, the resistance R₂ is increased and the main volume flow goes from node N₂₄ to node N₀₃₄ and from node N₀₃₄ to node N₁₃. If the microfabricated valve v₁ (Vm2) is not fully closed, the volume flow at the trapping position might go from N₀₁₂ to N₀₃₄ and the volume flow is not reversed. An immobilized matrix remains within its position. If the microfabricated valve v₁ (Vm2) is fully closed, a small volume flow might go from N₀₃₄ to N₀₁₂ with Qᵣₑᵥₑᵣₛₑ being smaller than Q_{crit}. Thus, an immobilized matrix remains within its position.
3. Only valve v₂ (Vn2) is actuated while v₁ (Vm2) is not actuated: In terms of this condition, the resistance R₃ is increased and the main volume flow goes from node N₂₄ to node N₀₁₂ and from node N₀₁₂ to node N₁₃. If the microfabricated valve v₂ (Vn2) is not fully closed, the volume flow at the trapping position might go from N₀₁₂ to N₀₃₄ and the volume flow is not reversed. An immobilized matrix remains within its position. If the microfabricated valve v₁ (Vm2) is fully closed, a small volume flow might go from N₀₃₄ to N₀₁₂ with Qᵣₑᵥₑᵣₛₑ being smaller than Q_{crit}. Thus, an immobilized matrix remains within its position.
4. Both valves v₁ (Vm2) and v₂ (Vn2) are actuated: In terms of this condition, the resistance R₂ as well as the resistance R₃ are increased and the main volume flow goes from node N₂₄ to node N₀₃₄, from node N₀₃₄ to node N₀₁₂ and from node N₀₁₂ to node N₁₃. Thus, a reverse flow is generated at the trapping position that might have a flow rate of Qᵣₑᵥₑᵣₛₑ larger than Q_{crit}. Thus, an immobilized matrix is removed from its position and moves via node N₀₁₂ to node N₁₃.

In another embodiment, various types of objects may be positioned within a RFCP-geometry and retrieved as disclosed in the present disclosure. In a particular embodiment, said objects may be biological cells, such as prokaryotic and/or eukaryotic cells, in particular cells of the immune system, cells related to different types of cancer, cells of the nerve system, stem cells. In another advantageous embodiment, said objects may be cell aggregates, in particular embryonic bodies and or spheroids composed of different cell types. One of the main advantages of positioning cells within a RFCP-geometry is that cells might be first characterized when immobilized within a RFCP-geometry and subsequently sorted using the disclosed retrieval mechanism represented by a generation of a reverse flow.

In another embodiment, the advantage of positioning matrices containing cells within an RFCP-geometry is that single and/or multiple cells can be cultivated and observed for an extended time period in a highly defined microenvironment that is provided by the matrix. In another embodiment, matrices may contain biological compounds, in particular proteins, in particular antibodies, antibody-DNA conjugates, extracellular matrix proteins, growth factors, nucleic acids, in particular DNA, RNA, PNA, LNA, lipids, cytokines, chemokines, aptamers as well as metabolic compounds, chemical compounds, in particular small molecules, in particular drugs, molecules linked via photocleable spacer/linker, nanostructures, in particular gold nanoparticles, growth promoting substance, inorganic substances, isotopes, chemical elements.

The advantage of the RFCP geometry is that immobilized matrices might be trapped and removed in a reversible manner by controlling the corresponding valve positions. In addition, as the removal process is based on a reverse flow, the removal process is cell compatible and very gentle in comparison to other methods (such as the use of a higher temperatures for generation of bubbles or for the degradation of said matrices) which is critical for handling single cells or small cell populations. In addition, the removal process maintains the integrity of immobilized matrices which is critical if said matrices store any information (e.g. secreted analytes bound to probes immobilized within said matrices) that might be accessed at later stage.

Removing a matrix from position n,m. In another advantageous embodiment, multiple RFCP geometries might be arranged within an n×m array whereas a matrix located at position (n,m) might be specifically removed from said array with a dramatic reduction in the number of actuators needed for removing said matrix. To this end, the microfabricated valves v₁ from all RFCP geometries located in row n might be actuated by a first actuator Aₙ and the microfabricated valves v₂ from RFCP geometries located in column m might be actuated by a second actuator Aₘ (said actuators might be pneumatic solenoid valves). Thus, if an actuator Aₙ as well as an actuator Aₘ is actuated, only at position (n,m) both microfabricated valves v1 and v2 from the RFCP geometry are closed/actuated resulting in a removal of a matrix immobilized at this position as described previously. Multiple microfabricated compartments having a RFCP geometry might be perfused with the same fluid by connecting said microfabricated compartments at node N₂₄ in a way that the same hydrodynamic pressure p1 is applied to all microfabricated chambers. In addition, all nodes N₁₃ from said microfabricated chambers might be connected so that all microfabricated compartment have the same hydrodynamic pressure p₂ at node N₁₃. Thus, matrices that are removed using said RFCP geometry might move to a common microfabricated channel which might be defined as collection channel. Said collection channel might be connected to a common outlet that enables the transfer of removed matrix into another format. This has the advantage that any position (n,m) within said array having n×m positions can be addressed by using only n+m actuators instead of n×m actuators.

Removing multiple matrices simultaneously. In another advantageous embodiment, multiple positions within said n×m array might be addressed simultaneously. For example, a first actuator Aₙ₁, a second actuator Aₙ₂ and a third actuator Aₘ₁ might be actuated simultaneously. This leads to a simultaneous removal of matrices located at the positions (n₁, m₁) and (n₂, m₁). The simultaneous removal of immobilized matrices has the advantage that the time needed for removing said matrices is dramatically removed.

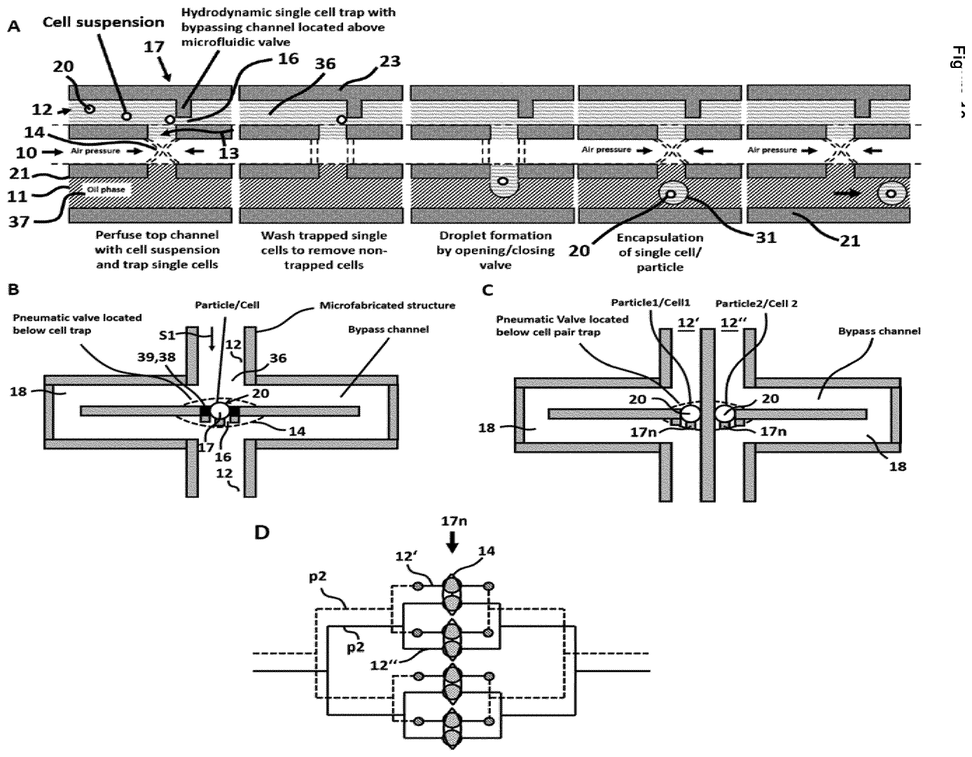

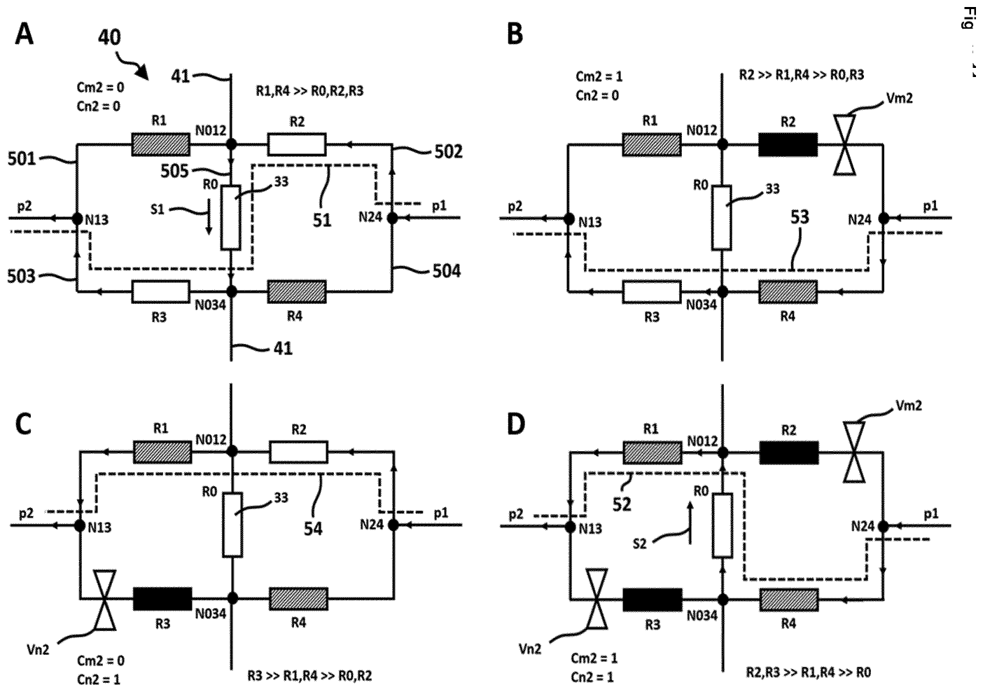

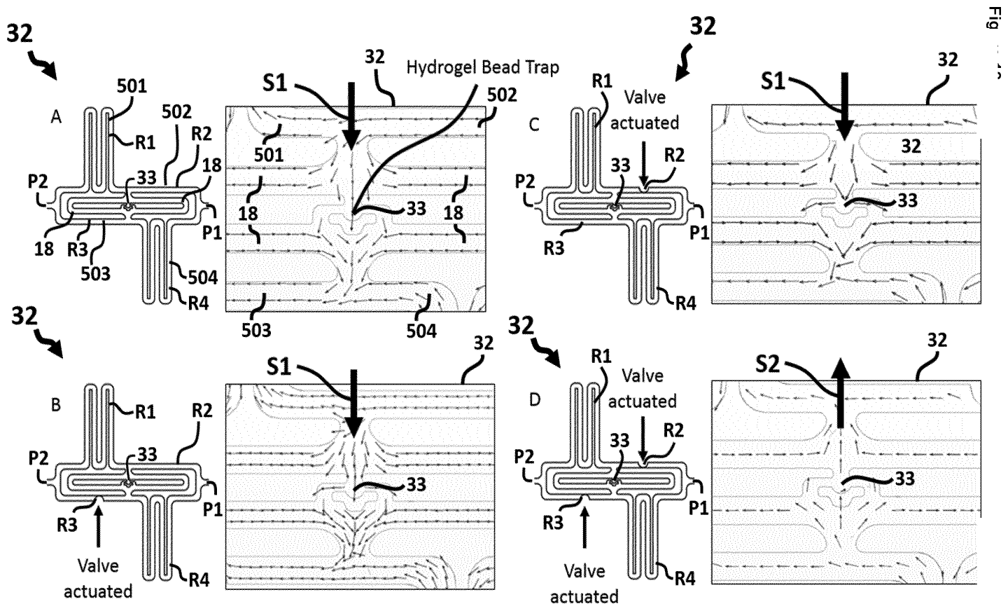

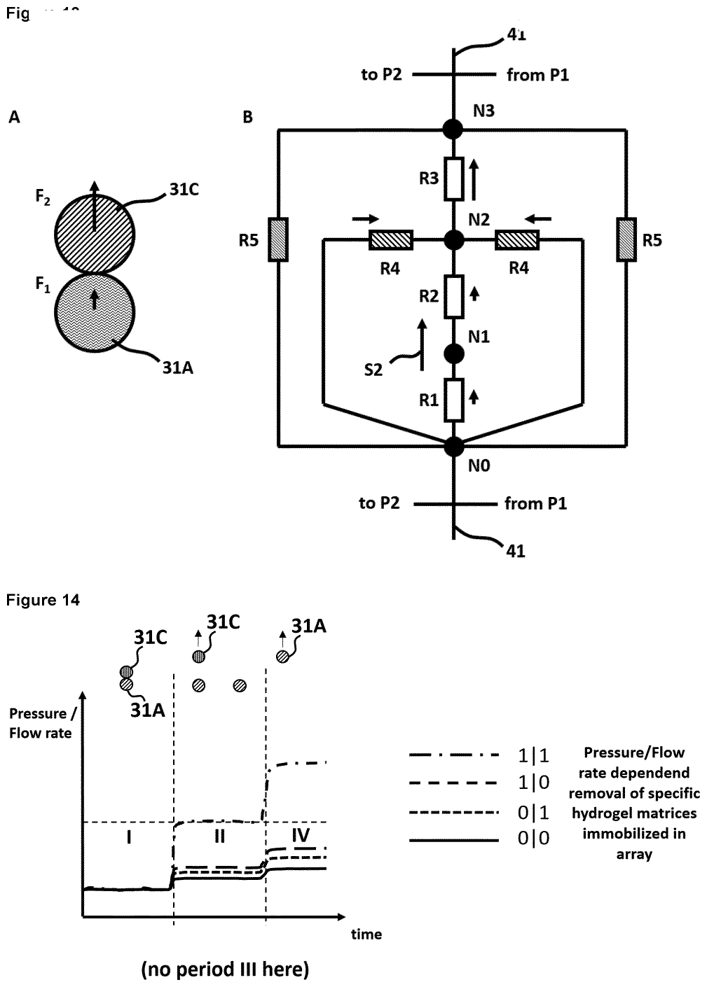

Immobilization and removal of two matrices. In another advantageous embodiment, two matrices of the same or of different type that are located at a certain position (n,m) within a microfabricated compartment which is part of a RFCP geometry might be sequentially removed (FIGS. 13 and 14). To this end, two matrices are positioned in close proximity or in contact within a microfabricated compartment. Said microfabricated compartment might have a bypass channel with the hydrodynamic resistance R_{bypass}=2×R₅ as well as a microfabricated geometry for the immobilization of two matrices having the resistance R_{Trapping Geometry}=R₃+(R₄⁻¹+R₄⁻¹+(R₁+R₂)⁻¹)⁻¹ (FIG. 13). During the immobilization of matrices, the main volume flow might flow from node N3 to N0 through the hydrodynamic resistance R_{Trapping Geometry} as R_{Trapping Geometry} might be smaller than the resistance of the bypass channel R_{bypass}. If a first enters the trapping geometry, the hydrodynamic resistance R_{Trapping Geometry} increases but remains smaller than the resistance of the bypass channel. Thus, a second matrix entering said microfabricated trapping geometry enters the trapping geometry and the hydrodynamic resistance of said trapping geometry increases so that R_{Trapping Geometry}>>R_{bypass}. A third matrix might enter the bypass channel and move to the next microfabricated compartment. Due to the described hydrodynamic resistances, applying a reverse flow results in a force acting on the trapped matrices with a force F₁ acting on matrix 1 (31A) positioned at node N₁ and with a force F₂ acting on matrix 2 (31C) positioned at node N₂ with F₁<F₂. A critical force F_{crit,n} might be needed to remove a matrix n located at position n within a microfabricated compartment. For example, F_{crit,1} is the force necessary to remove a matrix located at position 1 and F_{crit,2} is the force necessary to remove a matrix located at position 2. The forces acting on said matrices dependent on the applied pressure difference between the nodes N₃ and N₄. If all matrices have to experience the same force F_{crit} to be removed from the microfabricated trapping geometry the reverse flow rate for removing matrix 2 may be increased until F₂ equals F_{crit}. The force acting on the matrices 1 and 2 is F₁ and F₂ respectively with F₁<F₂ and F₁<F_{crit}. Thus, only the matrix 2 is removed while matrix 1 stays within its position. A further increase of the flow rate and thus the pressure difference might result in a force F₁ acting on matrix 1 that equals F_{crit} which leads to a removal of matrix 1.

This has the main advantage that immobilized matrices can be removed sequentially. For example, a matrix located at position 2 might be removed and collected within a first well of a 96-well plate or another format. Afterwards, a matrix located at position 1 might be removed and collected within a second well. Another advantage is that one matrix might be paired with various second matrices in a sequential manner. For example, matrix of type 1 might first be positioned next to matrix of type 2. Matrix of type 2 might be removed after a certain period and a new matrix might be positioned next to matrix of type 1. This process might be repeated several times.

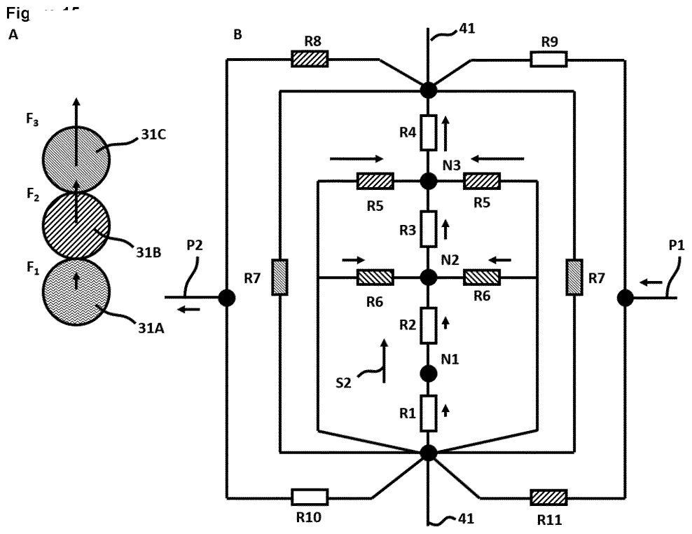

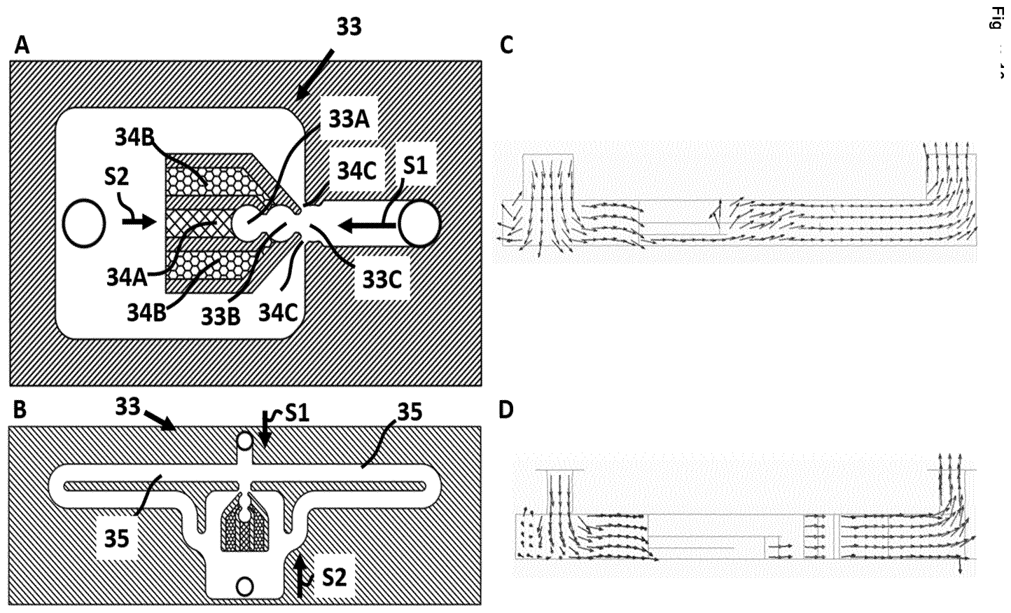

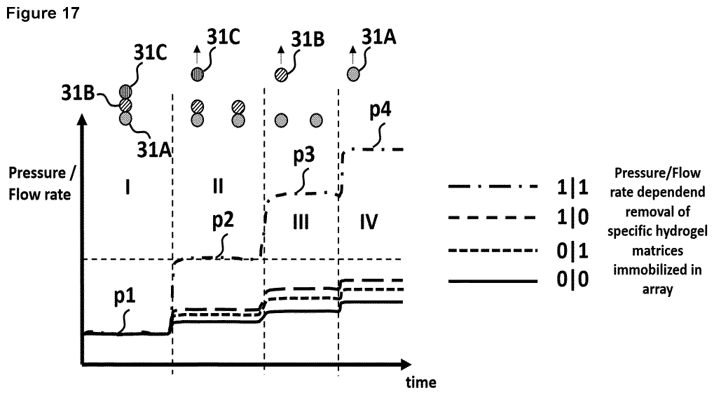

Immobilization and removal of three matrices. In another advantageous embodiment, three matrices of the same or of different type that are located at a certain position (n,m) within a microfabricated compartment which is part of a RFCP geometry might be sequentially removed. To this end, matrices might be first immobilized as described previously (FIG. 15, FIG. 16 and FIG. 17). Applying a reverse flow results in a force acting on the trapped matrices with a force F1 acting on matrix 1 (31A) positioned at node N1 and with a force F2 acting on matrix 2 (31B) positioned at node N2 with F1<F2. Applying a reverse flow results in a force acting on the trapped matrices with a force F1 acting on matrix 1 positioned at node N1, with a force F2 acting on matrix 2 positioned at node N2 and with a force F3 acting on matrix 3 (31C) positioned at node N3 with F1<F2<F3. Applying a reverse flow results in a force acting on the trapped matrices with a force F1 acting on matrix 1 positioned at node N1, with a force F2 acting on matrix 2 positioned at node N2 and with a force Fn acting on matrix n positioned at node Nn with F1<F2< . . . <Fn. A critical force Fcrit,n is needed to remove a matrix n located at position n within a microfabricated compartment. The forces acting on said matrices dependent on the applied pressure difference. If all matrices have to experience the same force Fcrit to be removed from the microfabricated trapping geometry the reverse flow rate for removing matrix 3 may be increased until F3 equals Fcrit. The force acting on the matrices 1 and 2 is F1 and F2 respectively with F1<F2<F3 and F1<F2<Fcrit. Thus, only the matrix 3 is removed while matrix 1 and matrix 2 stay within their position. A further increase of the flow rate might result in a force F2 acting on matrix 2 that equals Fcrit which leads to a removal of matrix 2 while matrix 1 stays in place. Finally, a further increase of the flow rate might result in a force F1 acting on matrix 1 which is equal to Fcrit. Thus, the matrix 1 is removed. This has the main advantage, that immobilized matrices can be removed sequentially. For example, a matrix located at position 3 might be removed and collected within a first well of a 96-well plate or another format. Afterwards, a matrix located at position 2 might be removed and collected within a second well.

Immobilization and removal of more than three matrices. In another advantageous embodiment, more than three matrices of the same or of different type that are located at a certain position (n,m) within a microfabricated compartment which is part of a RFCP geometry might be sequentially removed. To this end, matrices might be first immobilized as described previously so that multiple matrices might be positioned in a sequence. Said matrices might be located within a microfabricated trapping geometry in which each matrix experiences a different force deepening on its trapping position. Applying a reverse flow results in a force acting on the trapped matrices with a force F₁ acting on matrix 1 positioned at node N₁, with a force F₂ acting on matrix 2 positioned at node N₂ and with a force Fₖ acting on matrix k positioned at node Nₖ with F₁<F₂< . . . <Fₖ. A critical force F_{crit,k} might be needed to remove a matrix k located at position k within a microfabricated compartment. The forces acting on said matrices dependent on the applied pressure difference. If all matrices have to experience the same force F_{crit} to be removed from the microfabricated trapping geometry the reverse flow rate for removing matrix k may be increased until Fₖ equals F_{crit}. The force acting on the matrices 1, 2 . . . k is F₁, F₂ . . . Fₖ respectively with F₁<F₂< . . . <Fₙ and F₁<F₂< . . . <F_{crit}. Thus, only the matrix k is removed while all matrices 1, 2 . . . k−1 stay within their position. A further increase of the flow rate might result in a force Fₖ₋₁ acting on matrix k−1 that equals F_{crit} which leads to a removal of matrix k−1 while matrix k−2 stays in place. Finally, this process might be repeated until all matrices have been removed. This has the main advantage, that multiple immobilized matrices can be removed sequentially and transferred into a 96-well plate or another format.

Extraction of cells located within immobilized matrices and subsequent transfer into another format—Highly controlled cell transfer using RFCP. In another advantages embodiment, said array might be used to transfer single or multiple cells located within a matrix that is positioned within said array to another format such as a 96-well plate, a 384-well plate, a 1536-well plate or a microwell plate whereas exactly one single cell might be transferred to a pre-defined well of said established formats or each similar formats. For example, a matrix might contain initially one single cell. After cultivation for a certain time period (e.g. 3 days) said single cell might divide and proliferate and might form a spheroid consisting of more than one. The encapsulated cells might be separated from each other and subsequently transferred into another format whereas each well of said format will only contain one single cell derived from said matrix. For example, said extraction process might be performed in the following steps:
1. Immobilization of cell-laden matrices. Immobilization of matrices containing single or multiple cells within a positioner, in particular trapping structure at which the flow can be reversed using the previously mentioned RFCP mechanism.
2. Optionally: Cell cultivation within matrices. Cultivation of cells for an extended time period (For example cells might be cultivated for one, two or more than three days up to several weeks).
3. Event-triggered removal of immobilized matrices. As soon as a certain event occurs, the matrix containing said cells is removed from the trap by said RFCP mechanism and transferred to a perfusion compartment containing a filter structure that holds the matrix in place and allows smaller matrix to pass through. For example, said event might be a certain fluorescence intensity of the cultivated cells (e.g. cultivated cells might express a fluorescent reporter protein), a certain cell morphology such as an increased cell size, the formation of a cell spheroid with a certain size or a certain surface profile.
4. Extraction of single cells from matrices. The cell-laden matrix that is hold in place at the filter structure is then perfused with a solution that enables the separation of aggregated cells that might be attached due to cell-cell or cell-matrix contacts. Said solution might contain for example a protease (e.g. trypsin) for digesting surface proteins that mediate cell-cell contacts as well as cell-cell and cell-matrix adhesion. Afterwards, the matrix that contains now separated cells is dissolved. In particular, this might be done by perfusion with metalloproteases for that contain degradation sites that can be cleaved by metalloproteases for digestion.
5. Refocussing of single cells. Cells that are released from the matrix due to matrix removal are further separated from each other by using a re-focusing geometry or by using multiple re-focusing geometries in sequence.
6. Trapping within RFCP geometries. Re-focused cells might be trapped in a single cell trap located within a RFCP geometry. Multiple RFCP traps might be positioned in sequence connected with each other.
7. Transfer of trapped single cells into a standard format. Afterwards, single cells located with said RFCP geometries might be transferred to a standard format such as the well by actuating the corresponding valves as described previously.

This has the advantage that cells derived from one single cells can be separated and further analysed with conventional methods such as RT-PCR or single-cell sequencing without losing the time-lapse information about the cultured cells that has been recorded during cell culture. For example, this time-lapse information might be among others growth data, fluorescence data or migration data.

Use of a Cell Culture Plate

According to one embodiment, the cell-laden matrix is provided in a cell culture plate. Details of the cell culture plate (e.g. a 12, 24, 96, 384 etc. well-plate) and the cell-laden matrix are described elsewhere herein. In particular, the matrix may be provided by a hydrogel. The matrix may be three-dimensional and e.g. at least partially ellipsoidal, preferably plug or semi-sphere shaped. In one embodiment, a cell-laden matrix comprising at least one cell is provided per compartment (e.g. well) of the cell culture device. The cell-laden matrix may comprise more than one cell. Examples are a cell colony or one or more different cell types as disclosed herein. In one embodiment, the cell-laden matrix is provided in the compartment such that liquid that may surround the cell-laden matrix can be removed or exchanged without affecting the cell-laden matrix.

In particular, the cell-laden matrix is provided by a three dimensional hydrogel comprising more than one cell, wherein the cell-laden matrix is provided in the compartment such that liquid that may surround the cell-laden matrix can be removed or exchanged without affecting the cell-laden matrix. A single cell-laden matrix may be provided per compartment that comprises a cell-laden matrix. The cell-laden matrix may for instance prepared by transferring a solution comprising the matrix material and the at least one cell into the compartment of the cell culture plate. After transferal, the solution forms the matrix generating the cell-laden matrix.

In one embodiment, the cell-laden matrix is incubated in a compartment of said cell culture plate, e.g. a well of a well plate. The cell-laden matrix in the compartment of the cell culture plate may be surrounded at least partially by a liquid. The liquid can be present during the incubation. The liquid preferably covers the cell-laden matrix completely during incubation in order to avoid drying of the matrix. Liquids suitable for incubation of the cell-laden matrix have been described herein and it is referred thereto. In a particular embodiment, the cell-laden matrix in the compartment is covered by cell culture media.

Incubation of the cell-laden matrix optionally takes place before being in fluidic contact with the capture matrix. The released one or more biomolecules of interest may thus accumulate in the compartment, in particular in the liquid comprised in the compartment.

A suitable incubation period in particular depends on the cell type comprised in the cell-laden matrix and can be determined by the skilled person in relation to the used cells and the biomolecule(s) of interest. A suitable incubation period can be selected in embodiments from the range of 1 h to 72 h, such as 4 h to 72 h. A shorter incubation period (e.g. 1 h to 24 h) may be selected for microbiological applications. For instance, a shorter incubation period may be selected for a prokaryotic cell, such as a bacterial cell, which can be comprised in the cell-laden matrix as disclosed herein. A longer incubation period (e.g. 4 h to 72 h) may be selected for other applications. For instance, a longer incubation period may be selected for a eukaryotic cell, such an animal cell, which can be comprised in the cell-laden matrix.

After incubation, the capture matrix may be added to the compartment for binding the released biomolecule(s) of interest. Other contacting orders are also within the scope of the present disclosure as described herein. Furthermore, the liquid in the compartment comprising the one or more biomolecules of interest can be obtained and added to the capture matrix. In such embodiment, the capture matrix may also be present in a different compartment. In one embodiment, more than one capture matrix is provided and contacted with the one or more cell-laden matrix, respectively the released biomolecule(s) of interest. Capture matrices have been described elsewhere herein and it is referred to the respective disclosure. After binding the one or more biomolecules of interest, the capture matrix is optionally transferred to another compartment, such as a compartment of a cell culture plate. The capture matrix may also be introduced into a microfabricated cell culture device as described herein.

Afterwards, the remaining cell-laden matrix inside the compartment of the cell culture plate can be incubated again and a fresh capture matrix may be added. This advantageously allows to acquire time-lapse secretion profiled of biomolecules of interest, as disclosed herein. Suitable time intervals for measurement have been disclosed herein and it is referred thereto.

Steps c) and d) and optionally step e) of the method according to the present disclosure are then performed as described elsewhere herein. After step c) unbound free detection molecules can be removed by washing.

Transfer of a Capture Matrix

According to an aspect of the invention, the method comprises following features:
Quantity and specificity information is added during or after detection molecule production (e.g. commercially available antibodies)
After incubation and binding the one or more biomolecule of interest, the capture matrix is transferred to a second compartment (e.g. of the microfabricated cell culture device). Hence, processing of the capture matrix is separated from cell cultivation.
Position information is added at the collection position (e.g. within a collection well).
Sample preparation and handling of capture matrices is performed utilizing a cell culture device, e.g. a microfabricated cell culture device. Said cell culture device enables to combine all different information within one barcode label that can be sequenced.

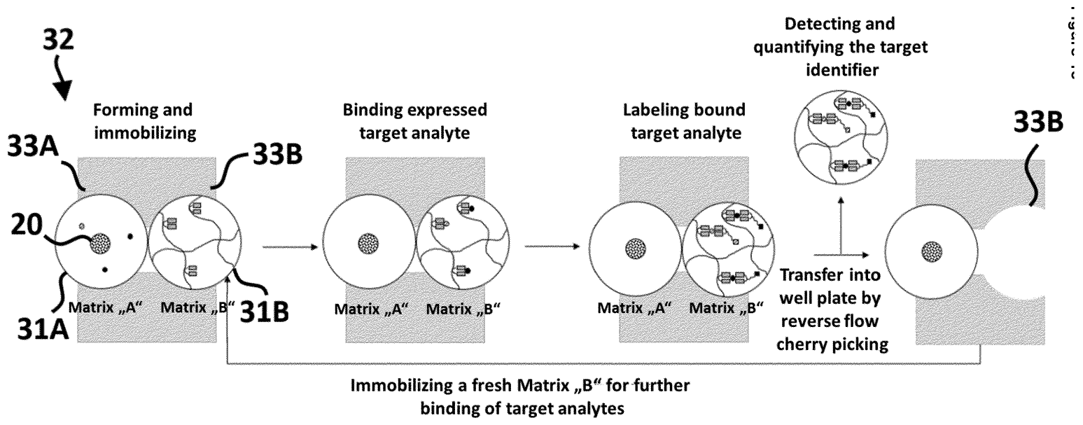

In another advantageous embodiment, the processing of the capture matrix is separated from the cell-laden matrix thereby preventing any (side-)effects on cell(s) located within said cell-laden matrices. To this end, a capture matrix and at least one cell-laden matrix are incubated within a first closed compartment as described before. After a defined incubation time (e.g. 1 h, 2 h or more), the first compartment is selectively opened and the capture matrix is transferred to a second compartment e.g. by using one or more microfabricated valves, preferably by a device comprising a valve arrangement which is adapted to selectively change the direction of fluid passing the location (e.g. RFCP geometry). The second compartment contains a positioning mean (e.g. a hydrodynamic trap) located within the compartment for trapping of the capture matrix and subsequent controlled transfer into another format. Thus, in one exemplary embodiment the exit portion of the first compartment is connected to the feeding channel of the second compartment as illustrated exemplary in FIG. 20. The second compartment can subsequently be perfused with various solutions without influencing the first compartment which still contains single or multiple cell-laden matrices. Thus, the capture matrix containing one or more types of capture molecules and thereto bound biomolecules of interest are first transferred to the second compartment, where the capture matrix is then perfused e.g. with different solutions such as PBS, a blocking solution, a solution containing detection molecules comprising a barcode label, a solution containing one or more oligonucleotides e.g. comprising a barcode for the current time point/UMI sequence, solution for performing a polymerization extension reaction.

The separation of the capture matrix processing and the position of the cell-laden matrices offers several advantages. Firstly, the cell-laden matrix does not come into contact with any solutions or buffers that might influence cell behaviour. Secondly, if cell(s) located within said matrix continue to release biomolecules after said incubation time, said biomolecules cannot be quantified during the processing of the capture matrix as the compartment has to be perfused with different solutions thereby washing away any additionally released biomolecules. Thirdly, a new capture matrix that does not have bound any biomolecules of interest can be positioned next to the cell-laden matrix, as soon as the capture matrix having bound thereto the biomolecules of interest is removed.

### Further Embodiments

Further general characteristics and embodiments are disclosed in the following.

According to another embodiment, the method can be conducted utilizing a microfabricated cell culture device and/or a collection position, which is preferably a collection well (e.g. from a well plate). Also, mixtures of both can be utilized, for instance by performing method steps partially on the microfabricated cell culture device and partially at a collection position.

According to one embodiment, the method according to the first aspect can have one or more of the following characteristics:
i) the method measures and optionally, quantifies biomolecules secreted by at least one cell;
ii) capture of molecules is performed during cultivation/incubation of at least one cell; analysis of captured molecules is either performed during cultivation/incubation or afterwards
iii) a time-dependent analysis of secreted biomolecules is performed, wherein the method allows analyzing multiple biomolecules at multiple time points;
iv) biomolecules are analyzed time-dependently, wherein time points are selected from 1 to 100 or more, preferably 2 to 90, 3 to 80, 4 to 70, 5 to 60, or 5 to 50, more preferably 6 to 40, 7, to 30, or 8 to 20;
v) biomolecules are analyzed time-dependently, wherein the time interval between analyses is selected from ≥10 min, ≥20 min, ≥30 min, 1 h, ≥2 h, ≥3 h, ≥4 h, 5 h or more, up to days 1 d, 2 d or several days;
vi) the capture matrix and the cell-laden matrix are incubated within the same compartment
vii) after incubating the cell-laden matrix for a pre-defined period, the capture matrix is added to the compartment, wherein the cell-laden matrix is surrounded by a water-immiscible fluid layer;
viii) after capturing the biomolecules of interest, molecules that have not bound to the one or more types of capture molecules are removed which may be done by flushing the compartment with a fluid;
ix) at least steps a) to (c are performed more than one time, preferably ≥2 times, ≥3 times, ≥4 times, ≥5 times, ≥6 times, 7 times, ≥8 times; and/or
x) the method is performed in an automated manner.

Coupling of Phenotypic and Genotypic Information

Another advantage of said disclosure is that cells can be cultivated over an extended period at n×m positions. During the cultivation period, released molecules can be captured and processed and subsequently analysed and quantified. In addition, cells can be removed from positions n×m at any time point and as soon as a defined requirement is fulfilled. Afterwards, removed cells might be analysed with conventional methods such as qRT-PCR or sequencing. Thus, a further critical advantage is the coupling of various cell specific data including:
Phenotypic data, such as:
   time-lapse microscopy data such as fluorescent data that can be gained for example by using cells expressing a fluorescent reporter molecule or by using fluorescent probes such as live cell membrane stainings. In addition, data such as the cell shape, cell migration and cell viability (e.g. formation of apoptotic bodies), formation of lamellipodia, may be derived from the time-lapse microscopy data to gain more information about the cell phenotype.
   Time-lapse secretion profiles gained as disclosed
   Surface marker profiles
   Intracellular phenotypic data gained by using techniques such as immunostaining
Genotypic data for example gained from techniques such as qRT-PCR or sequencing

For example, a single cell located within a cell-laden matrix at position (n, m) might express a fluorescent protein that is coupled to a specific promotor. The single cell might start to proliferate resulting in a small cell colony. In addition, during the cell cultivation, the cell may release various molecules (e.g. via secretion) that can be analysed using the current disclose. As soon as the fluorescent signal of said colony reaches a certain value the matrix located at position (n, m) containing said colony might be removed and analyzed with qRT-PCR or NGS. Thus, the current disclose provides the unique advantage to combine various time-lapse phenotypic data with the underlying genotype on a single-cell level and is applicable to hundreds to thousands of cell simultaneously.

In another embodiment, previously described methods can be performed within an array of compartments, provided by a microfabricated cell culture device. This enables the simultaneous determination of time-lapse secretion profiles of (single) cell(s) located in hundreds to thousands of compartments.

One compartment for incubation and detection bead processing. To this end, multiple compartments (comprised in the cell culture device, preferably the microfabricated cell culture device) are connected in series sharing a common feeding line that is used for the delivery of capture matrices and cell-laden matrices as disclosed in PCT/EP2018/074527. In addition, each compartment can be perfused individually without affecting other compartments by using a perfusion line. An exemplary embodiment is described in the present disclosure. It is furthermore referred to the following Figure of PCT/EP2018/074526, including the corresponding figure description, which both are herein incorporated by reference:
FIG. 2. The figure illustrates the structure of a generic array that can be used for the handling (e.g. positioning, incubation and removal) of capture matrix and cell-laden matrices (Array of RFCP geometry)

Thus, the feeding line is used for initial loading of the multiple compartments with cell-laden matrices, as well as with capture matrices. Afterwards, the compartments are selectively closed for generating an isolated compartment and thus a defined reaction volume. The processing of the capture matrix can be performed using the perfusion line. Thus, the required different solutions (such as detection molecule solution, washing solution, etc.) can be delivered using the perfusion line or the feeding line.

Removal of a capture matrix located at a defined position can be performed using a valve arrangement which is adapted to selectively change the direction of fluid passing the location (e.g. RFCP mechanism) as disclosed in PCT/EP2018/074527 by addressing the corresponding row and column valves. Capture matrices that do not have bound any biomolecule(s) of interest can be delivered again via the feeding line (e.g. all compartments are perfused with a solution containing capture matrices that do not have bound thereto any biomolecules of interest).

Two Compartments, One for Incubation and One for Capture Matrix Processing.

If the capture matrix processing is spatially separated from the location at which the cell-laden matrices are positioned, two RFCP geometries can be connected and arranged within an array. An illustration of the structure of an array containing separated RFCP geometries, one for processing the capture matrix (processing chamber) and one for cell culture and binding of the biomolecules of interest (e.g. microfluidic cell culture compartment) is given in FIG. 20. The separation of the capture matrix processing from the compartment containing the cell-laden matrix/matrices is advantageous as it prevents that the processing of the capture matrix influences cell(s) located within the cell-laden matrices.

The matrices for cell encapsulation and biomolecule if interest capture might have the same or different sizes and might be composed of the same material or a different material.

In one embodiment, both matrices might have a spherical shape with a diameter of 80 μm.

In another aspect of the invention, a method for detecting a plurality of biomolecules of interest, in particular proteins, secreted from a single cell is disclosed.

### The Kit According to the Second Aspect

According to a second aspect, the present disclosure provides a kit comprising:
a) one or more types of detection molecules, wherein each type of detection molecule specifically binds a biomolecule of interest, and wherein each type of detection molecule comprises a barcode label which comprises a barcode sequence (B_{S}) indicating the specificity of the detection molecule; and
b) at least one oligonucleotide, optionally a primer, that is preferably capable of hybridizing to the barcode label of the at least one type of detection molecule.

Such kit can be used e.g. in the method according to the first aspect. The one or more types of detection molecules and the at least one oligonucleotide have been disclosed above in detail for the method of the first aspect and it is here referred to the respective disclosure which also applies here. The same applies with respect to the further sequence elements, adaptor barcode oligonucleotide, primer and primer combinations, the barcode label, set of oligonucleotides, matrices, and other optional kit components.

Preferably, the kit comprises at least one oligonucleotide that is capable of hybridizing to the barcode label of the at least one type of detection molecule. An example is a primer and furthermore the adaptor barcode oligonucleotide disclosed herein. Alternatively, the kit may comprise an oligonucleotide that may be ligated to the barcode label of the detection molecule to provide an extended barcode label.

According to one embodiment, the oligonucleotide of said kit comprises at least one sequence element selected from the group consisting of

(i) a barcode sequence (B_{T}) for indicating a time information,

(ii) a barcode sequence (B_{P}) for indicating a position information, and

(iii) a unique molecular identifier (UMI) sequence.

The kit furthermore may have one or more of the following features:
a) it comprises an adaptor barcode oligonucleotide capable of hybridizing to the barcode label of at least one type of detection molecule, wherein the adaptor barcode oligonucleotide comprises 5′ to the region that is capable of hybridizing to the barcode label (i) a barcode sequence (B_{T}) for indicating a time information, a barcode sequence (B_{P}) for indicating a position information, and/or (ii) a unique molecular identifier (UMI) sequence;
b) it comprises an adaptor barcode oligonucleotide, wherein the adaptor barcode oligonucleotide comprises an adaptor sequence (1)R that is reverse complementary to an adapter sequence (1) of the barcode label of the detection molecule, wherein the adaptor barcode oligonucleotide additionally comprises at least one, at least two, at least three or all sequence elements selected from the group consisting of
   a barcode sequence (B_{T}) for indicating a time information,
   a barcode sequence (B_{P}) for indicating a position information,
   a unique molecular identifier (UMI) sequence, and
   a primer target sequence,
   wherein these one or more sequence elements are located 5′ of the adaptor sequence (1)_{R};
c) a primer or primer combination comprising one or more of the following
   a barcode sequence (B_{P}) for indicating position information,
   a barcode sequence (B_{T}) for indicating a time information,
   an adapter sequence (AS) for sequencing,
   wherein the one or more sequence elements B_{P}, AS, and/or B_{T} if included in the primer or a primer of the primer combination, are located 5′ of the sequence region of the primer that is capable of hybridizing to the optionally extended barcode label or the reverse complement thereof;
d) the barcode label of the one or more types of detection molecules comprises
   (i) the barcode sequence (B_{S}) indicating the specificity of the detection molecule;
   (ii) one or more primer target sequences;
   (iii) optionally a barcode sequence (B_{T}) indicating a time information;
   (iv) optionally a unique molecular identifier (UMI) sequence; and
   (v) optionally an adapter sequence (1).

According to one embodiment, wherein the kit comprises at least one set of oligonucleotides selected from the following group:
a) set 1 comprising:
   a. a barcode label attached to the detection molecule comprising:
      i. optionally a cleavable linker/spacer,
      ii. optionally a first primer binding sequence (1),
      iii. a barcode sequence B_{S},
      iv. an adaptor sequence (1);
   b. an adaptor barcode oligonucleotide comprising:
      i. an adaptor sequence (1)_{R},
      ii. a unique molecular identifier (UMI) sequence,
      iii. a second primer binding sequence (2)_{R},
   c. a forward primer comprising:
      i. a primer sequence (1),
      ii. a barcode sequence B_{P},
      iii. an adaptor sequence for sequencing (AS);
   d. a reverse primer comprising:
      i. a primer sequence (2)_{R},
      ii. a barcode sequence B_{T},
      iii. an adaptor sequence for sequencing (AS);
b) set 2 comprising:
   a. a barcode label attached to the detection molecule comprising:
      i. optionally a cleavable linker/spacer,
      ii. a first primer binding sequence (1),
      iii. a barcode sequence B_{S},
      iv. an adaptor sequence (1);
   b. an adaptor barcode oligonucleotide comprising:
      i. an adaptor sequence (1)_{R},
      ii. a barcode sequence B_{P},
      iii. a unique molecular identifier (UMI) sequence,
      iv. a second primer binding sequence (2)_{R};
   c. a forward primer comprising:
      i. a primer sequence (1),
      ii. a barcode sequence B_{T},
      iii. an adaptor sequence for sequencing (AS);
   d. a reverse primer comprising:
      i. a primer sequence (2)_{R},
      ii. an adaptor sequence for sequencing (AS);
c) set 3 comprising:
   a. a barcode label attached to the detection molecule comprising:
      i. optionally a cleavable linker/spacer,
      ii. a first primer binding sequence (1),
      iii. a barcode sequence B_{S},
      iv. an adaptor sequence (1);
   b. An adaptor barcode oligonucleotide comprising:
      i. an adaptor sequence (1)_{R},
      ii. a barcode sequence B_{T},
      iii. a unique molecular identifier (UMI) sequence,
      iv. a second primer binding sequence (2)_{R};
   c. A forward primer comprising:
      i. a primer sequence (1),
      ii. a barcode sequence B_{P},
      iii. an adaptor sequence for sequencing (AS);
   d. A reverse primer comprising:
      i. a primer sequence (2)_{R},
      ii. an adaptor sequence for sequencing (AS);
d) set 4 comprises:
   a. a barcode label attached to the detection molecule comprising:
      i. optionally a cleavable linker/spacer,
      ii. a first primer binding sequence (1),
      iii. a barcode sequence B_{S},
      iv. a unique molecular identifier (UMI) sequence,
      v. a barcode sequence B_{T},
      vi. a second primer binding sequence (2);
   b. a forward primer comprising:
      i. a primer sequence (1),
      ii. a barcode sequence B_{P},
      iii. an adaptor sequence for sequencing (AS);
   c. a reverse primer comprising:
      i. a primer sequence (2)_{R},
      ii. an adaptor sequence for sequencing (AS).

The kit may furthermore comprise two or more of such sets.

The kit furthermore may comprise
a) one or more types of capture molecules, wherein each type of capture molecule binds a biomolecule of interest, wherein preferably, the one or more types of capture molecules provided in the kit bind the same biomolecules of interest as the one or more types of detection molecules comprised in the kit;
b) one or more polymers for providing the matrix for the cells and/or the capture matrix, wherein preferably the polymer is capable of forming a hydrogel;
c) a composition, preferably a solution, containing capture matrices;
d) a polymerase and/or dNTPs; and/or
e) a wash solution.

According to one embodiment, the composition containing capture matrices, which preferably is a solution, may contain monodisperse capture matrices with a pre-defined size (e.g. 80+−5 μm) and concentration (e.g. 50 matrices/μL). Said capture matrices contain a pre-defined mix of immobilized capture molecules (e.g. one or more types of capture molecules). In addition, said capture matrices may have passed a quality control prior to distribution. During the quality control, capture matrices may have been validated in terms of one or more of the following parameters: binding capacity of analytes, respectively biomolecules of interest (e.g. 1×10⁶ analytes per capture matrix), elastic properties, dynamic range, detection sensitivity, multiplexing capability and cross-reactivity with different analytes. Different capture matrix solutions may be offered depending on the application. For example, said solution may contain capture matrices with capture molecules against II-6 and II-10 for studying interactions between immune cells and tumour cells. In another application, CCL-2 and CCL-5 specific capture matrices may be offered for studying the influence of chemoattractants.

The detection molecules may be comprised in a composition, such as a solution. It may contain at least one type of detection molecule comprising a barcode label. Embodiments are disclosed herein. The composition may comprise more than one type of detection molecule for performing a multiplex analysis for detecting two or more biomolecules of interest. Thus, said solution may contain detection molecules of different types with a defined concentration.

The wash solutions, such as washing buffers may be used for removing unbound analytes and detection molecules.

According to one embodiment, the kit comprises a device with a plurality of compartments, preferably a multi-well plate (e.g. 96, 384 or 1536 well plate). The compartments of the device may comprise at least one oligonucleotide, preferably an adaptor barcode oligonucleotide, and/or a primer or primer combination. Details of suitable adaptor barcode oligonucleotides, primers and primer combinations as well as suitable sets are described elsewhere herein and it is referred to the respective disclosure. The compartments may furthermore comprise reagents for performing an extension and/or amplification reaction.

The kit may comprise a device with a plurality of compartments, preferably a multi-well plate, wherein said device has one or more of the following characteristics. The device may be preloaded with reagents required for performing a primer extension and/or amplification step. Such device is particularly advantageous, if the introduction of the sequence elements UMI, B_{P} and/or B_{T} occurs separate from the cell-laden matrix that is comprised in the cell culture device. It is only necessary to transfer the capture matrices with the bound detection molecules into compartments of the device that is preloaded with reagents necessary for extending the barcode label and/or for performing an amplification reaction. In one embodiment, the compartments of the device comprise an oligonucleotide, preferably an adaptor barcode oligonucleotide and/or a primer or primer combination, as disclosed herein. Specifically, the compartments of the device may comprise at least one set selected from sets 1 to 4 as disclosed herein. The compartments may furthermore comprise reagents for performing an extension and/or amplification reaction, such as an enzyme mix comprising a reverse transcriptase (such as M-MuLV or AMV) and/or a polymerase (such as Taq DNA Polymerase) and optionally dNTPs. The reagents may be provided in lyophilized form in the compartments of the well. The device may be e.g. selected from a 96, 384 or 1536 well plate. The kit may furthermore comprise a solution for reconstitution of the lyophilized reagents. The kit may also comprise one or more reaction buffers and nuclease-free water.

### The Plurality of Sequenceable Products According to the Third Aspect

According to a third aspect, a plurality of sequenceable products is provided, wherein each sequenceable product comprises at least the following sequence elements

(i) a barcode sequence (B_{S}) for indicating a specificity, and

(ii) a barcode sequence (B_{T}) for indicating a time information, and/or

(iii) a barcode sequence (B_{P}) for indicating a position information, and

(iv) optionally a unique molecular identifier (UMI) sequence.

Such plurality of sequenceable products may be provided by the method according to the first aspect.

According to one embodiment, the sequenceable products of the plurality of sequenceable products differ from each other in one or more of the comprised sequence elements (i) to (iv).

According the number of sequenceable products comprising different sequence elements B_{S}, B_{T} and/or B_{P} is at least 50, preferably at least 100. As disclosed herein, the sequenceable products may additionally comprise unique UMI sequences.

The plurality of sequenceable products may comprise at least 2 different barcode sequences B_{S}, optionally wherein the number of different barcode sequences B_{S} may lie in a range of 2 to 100, 5 to 50, 5 to 25, 5 to 20 or 7 to 15. This advantageously allows to analyse multiple different biomolecules of interest in parallel.

The plurality of sequenceable products may comprise at least 2 different barcode sequences B_{T}, optionally wherein the number of different barcode sequences B_{T} may lie in a range of 2 to 200, 5 to 50, 5 to 25, 5 to 20 or 7 to 15. As disclosed herein, the method may be performed at different time points/time intervals, thereby allowing to generate a time-lapse profile of the biomolecules of interest.

The plurality of sequenceable products may comprise at least 2 different barcode sequences B_{P}, optionally wherein the number of different barcode sequences B_{P} may lie in a range of 2 to 1000, 5 to 1000, 10 to 500, 20 to 250 or 50 to 200. As disclosed above, it is possible to use a cell culture device comprising multiple different compartments (positions) comprising a cell-laden matrix, whereby a multiplex analysis of different cells, respectively cell-laden matrices located in different positions is possible. They can be distinguished based on the barcode label B_{P} indicating the position information and therefore allowing to correleate the result with a specific cell-laden matrix comprised in a compartment.

As disclosed herein, the UMI sequence may have a length of up to 40 nucleotides, preferably 4 to 20 nucleotides.

Such a plurality of sequenceable products can be generated and analysed using the method according to the first aspect. The sequence elements have been disclosed above in detail for the method of the first aspect and it is here referred to the respective disclosure which also applies here. The same applies with respect to the optional features of the plurality of sequenceable products.

As used herein, the term “comprising” is to be construed as encompassing both “including” and “consisting of”, both meanings being specifically intended, and hence individually disclosed embodiments in accordance with the present invention.

This invention is not limited by the exemplary method disclosed herein, and any methods, uses, systems and materials similar or equivalent to those described herein can be used in the practice or testing of embodiments of this invention. Numeric ranges are inclusive of the numbers defining the range. The headings provided herein are not limitations of the various aspects or embodiments of this invention which can be read by reference to the specification as a whole.

As used in the subject specification, the singular forms “a”, “an” and “the” include plural aspects unless the context clearly dictates otherwise. Reference to “the disclosure” and “the invention” and the like includes single or multiple aspects taught herein; and so forth. Aspects taught herein are encompassed by the term “invention”.

It is preferred to select and combine preferred embodiments described herein and the specific subject-matter arising from a respective combination of preferred embodiments also belongs to the present disclosure.

### DETAILED DESCRIPTION OF EMBODIMENTS ILLUSTRATED IN THE FIGURES

FIG. 1: FIG. 1 shows a microfluidic array 30 having a plurality of compartments (e.g. observation chambers 32), such a compartment 32m2n2 at position m2 n2, each loaded with (single) cell-laden matrix under perfusion culture. Depicted are the rows n and columns m of the array as well as corresponding compartments. Lines representing rows and columns are illustrating pressure lines for providing common group commands as is described herein. Circles illustrate individual compartments. Each compartment may contain at least one cell-laden matrix which can have defined characteristics. The matrix containing at least one cell may be provided by a hydrogel with defined characteristics (e.g. elasticity, immobilized ECM proteins and/or peptides, in particular RGD sites, fibronectin, YIGSR peptides, collagen, LDV peptides, laminin). The matrix preferably has a spherical form and may be provided by a hydrogel bead that contains at least one cell (e.g. an immune cell, a cancer cell, a stem cell).

FIG. 2A: Illustrates core steps of the method of the invention according to one embodiment:

As illustrated in A, a cell-laden matrix (1), which preferably is a hydrogel bead, and a capture matrix (2), which preferably is a hydrogel bead, are positioned in close proximity within an isolated compartment at a position X|Y of a device, which according to a preferred embodiment is a microfabricated cell culture device. The cell-laden matrix comprises in the illustrated embodiment a single cell (3), which secretes two biomolecules of interest (4a and 4b). The capture matrix (2) comprises in the illustrated embodiment two different types of capture molecules (5a and 5b), which specifically bind the biomolecules of interest (4a and 4b). The different types of capture molecules are in the illustrated embodiment provided by antibodies with different specificities against the secreted biomolecules of interest. The capture matrix preferably comprises a plurality of capture molecules of the same type to ensure efficient capture of a biomolecule of interest. The capture molecules may be provided in excess of the expected number of secreted biomolecule of interest.

In B, the cell-laden matrix (1) is incubated to allow sufficient secretion of the biomolecules of interest which diffuse from the cell-laden matrix (1) to the capture matrix (2), where a biomolecule of interest is bound by the matching type of capture molecule (see interaction pairs 4a/5a and 4b/5b). Unbound molecules may be washed away.

In C, one or more types of detection molecules are added, here two types of detection molecules (6a and 6b), wherein each type of detection molecule specifically binds a biomolecule of interest. Importantly, each type of detection molecule comprises a barcode label (7) which comprises a barcode sequence (B_{S}) indicating the specificity of the detection molecule. Thus, the specificity of the capture molecule can be determined based on the barcode label. The barcode label may be provided by an oligonucleotide sequence that may be attached via a linker to the detection molecule. In an embodiment, the linker is provided by a photocleavable spacer. In the illustrated embodiment the different types of detection molecules are provided by antibodies which bind the biomolecule of interest at a different epitope than the antibodies used for capturing. Thereby, a complex is formed, comprising the capture molecule, the biomolecule of interest and the detection molecule (see complex 4a/5a/6a and 4b/5b/6b).

In D, a sequenceable reaction product is generated which comprises at least (i) the barcode sequence (B_{S}), and (ii) a barcode sequence (B_{T}) for indicating a time information, and/or (iii) a barcode sequence (B_{P}) for indicating position information of the cell-laden matrix, and (iv) optionally a unique molecular identifier (UMI) sequence. The generation of the sequenceable reaction product comprises the use of at least one oligonucleotide, which in one embodiment is a primer, that is capable of hybridizing to the barcode label of the at least one type of detection molecule. As is described herein and also illustrated in the subsequent figures, step D may comprise several substeps, including transfer steps.

One embodiment of step D that is schematically illustrated in FIG. 2 comprises a step (aa), which is as described herein an optional, but in some embodiments a preferred step. Step (aa) comprises hybridizing an oligonucleotide (8) to the barcode label of the detection molecules and extending said barcode label by a polymerase reaction using the hybridized oligonucleotide as template, whereby an extended barcode label is obtained that remains attached to the detection molecule. The extended barcode label additionally comprises the sequence information of the hybridized oligonucleotide that was used as template. The oligonucleotide (in embodiments also referred to as adaptor barcode oligonucleotide) may comprise in embodiments explained in further detail below a barcode sequence (B_{T}) for indicating a time information (i.e. the current time point where the oligonucleotide is added) and/or a unique molecular identifier (UMI) sequence. Extension of the barcode label by a polymerase using the oligonucleotide as template transfers the barcode sequence (B_{T}) for indicating a time information and/or the UMI information from the oligonucleotide to the extended barcode label. As is illustrated in FIG. 2, the hybridized oligonucleotide may also be extended, whereby a double-stranded molecule (9) is generated. However, it is also within the scope of the present invention to use an oligonucleotide comprising a blocked 3-OH end that cannot be extended by a polymerase.

The capture matrix with the detection molecules, that comprise the barcode labels, which were optionally extended as described above in step D (aa), may be obtained from the compartment and can be transferred to a pre-defined compartment, such as a pre-defined well, of a different device. The transfer may occur using the RFCP-mechanism that is described elsewhere herein. The capture matrix with the (optionally extended) barcode labels may be e.g. transferred into a well of another format such as a 96-well plate. In embodiments, the transfer of the capture matrix occurs prior to step D, e.g. after capturing the biomolecules of interest in step B and/or after binding the detection molecules in step C. The removal of the capture matrix which comprises the complexes comprising the capture molecule, the biomolecule of interest and the detection molecule from the compartment leaves the cell-laden matrix in the compartment. As is illustrated in F, a “fresh” capture matrix may be added/loaded into the compartment and a new cycle may be performed at a different time-point. The steps may be repeated at several time-points.

Preferably, D comprises performing an amplification reaction using a primer or primer combination. In the illustrated embodiment, such amplification reaction is performed after performing step D (aa). The amplification reaction is indicated in FIG. 2 as D (bb) and comprises performing an amplification reaction with a primer or primer combination using the extended barcode label and/or the reverse complement thereof as template. Preferably, the extended barcode label is used as template (the reverse complement thereof may be removed as described elsewhere herein in case a double-stranded molecule is formed during the extension step that includes the reverse complement of the barcode label and/or an oligonucleotide with a blocked 3′-OH end may be used to prevent that a reverse complement strand of the barcode label is formed in the extension reaction). If a single primer is used, a linear amplification can be performed by performing several amplification cycles. The use of a primer combination such as a primer pair allows to perform a PCR reaction. The primer or primer combination as well as the additional components required for performing the amplification reaction (such as a polymerase, dNTPs, buffers) may be added to the compartments (e.g. wells) that comprise the transferred capture matrix or may be provided in advance. The primer or primer combination may comprise a barcode sequence (B_{P}) for indicating position information and optionally an adapter sequence (AS) for sequencing, e.g. a standard adapter for a sequencing platform. Further embodiments are illustrated in the subsequent figures. The primer or primer combination can hybridize to the optionally extended barcode label or the reverse complement thereof. As is described herein, the optionally extended barcode label may be released from the detection molecule in advance of the amplification reaction, e.g. when a photocleavable linker is used.

The amplification products may then be sequenced in step E. As is described herein, the method according to the present invention provides multiple pooling options, allowing to make the sequencing very cost and time efficient.

FIG. 2B illustrates examples of well positions in a well plate, into which the capture matrices can be transferred for performing the amplification reaction. As is described herein, the initial cell culture device may comprise several compartments for receiving a cell-laden matrix and a capture matrix. If the cell culture device comprises e.g. 100 compartments (cultivation positions for different cell-laden matrices) and 10 different types of capture molecules are used in combination with 10 different types of corresponding detection molecules to capture and detect the biomolecules of interest at 10 different time-points, 100 wells are required. As is shown in FIG. 2B, it is within the scope of the present invention to pool e.g. the capture matrices obtained from the same isolated compartment at time-points 1-10 (or the optionally extended barcode labels that are detached from the detection molecules) into a single well before performing the amplification reaction. This allows to amplify the optionally extended barcode labels obtained at the different timepoints 1-10 in a single amplification reaction. This is time and cost efficient. Moreover, as is described herein, the present method allows to introduce a barcode sequence B_{P} into the sequenceable reaction product. Thus, all sequenceable reaction products obtained at the different time-points comprise the same barcode sequence B_{P}, as these originate from the same cell-laden matrix comprised in an individual compartment. The barcode sequence B_{P} thereby allows to correlate the obtained sequenceable reaction products with the original compartment, respectively the comprised cell-laden matrix. E.g. the transfer of the capture matrix from the compartment of the cell culture device into the well of the device wherein the amplification is performed may be performed such that it allows to correlate the barcode sequence B_{P} with the original compartment of the in the cell culture device. As all sequenceable reaction products originating from the same compartment, respectively the same cell-laden matrix comprise the same barcode sequence B_{P}, their origin can be determined based on the barcode sequence B_{P}. This allows to pool all reaction products obtained after the amplification reaction in the different wells into a single pool/library that is then subsequently sequenced, preferably by NGS sequencing. Thus, the reaction products from all wells can be pooled and send for sequencing.

FIG. 2C illustrates an advantageous variation of the method illustrated in FIG. 2A. In the shown embodiment, the transfer of the capture matrix occurs after step C (i.e. after capturing the biomolecules of interest in step B and after binding of the detection molecules in step C) and prior to step D. At least one capture matrix that has captured the biomolecules of interest from at least one cell-laden matrix comprised in a compartment is transferred into a compartment of another device, such as a multi-well plate (e.g. a 96, 384 or 1536-well plate). According to one embodiment, exactly one capture matrix is transferred. The further capture matrix processing may be performed within this well. An UMI sequence may be introduced within said well using an adaptor barcode oligonucleotide comprising an UMI sequence. Thus, as only one capture matrix (or at least two capture matrices comprising the biomolecules of interest released from the same cell-laden matrix comprised in a compartment) is located within one processing/collection well, the required number of different UMIs (corresponds to the UMI library size) is significantly reduced and is limited to the maximum number of detection molecules that can be located within a capture matrix. This number corresponds to the maximum binding capacity of the used capture matrices. As the generation of large UMI libraries is costly, the reduction of the UMI library size results in a significant cost reduction. According to one embodiment, the further processing is performed as is disclosed and illustrated in FIG. 8a. According to a further preferred embodiment, the adaptor barcode oligonucleotide comprises an UMI sequence, while a barcode sequence B_{T} and a barcode sequence B_{P} is introduced during amplification using a primer or primer combination, wherein preferably, a primer combination is used wherein one primer comprises the barcode sequence B_{T}, while the other primer comprises the barcode sequence B_{P}. This embodiment is advantageous as it allows to provide the reagents and in particular the adaptor barcode oligonucleotides and the primer or primer combination pre-loaded (e.g. in lyophilized form) in the compartments (e.g. wells) of the device into which the capture matrices are transferred. The transfer occurs while maintaining/correlating the position information with the cell-laden matrices comprised in the cell culture device, so that the finally obtained results can be assigned to a cell-laden matrix, respectively the one or more cells comprised therein.

FIG. 2D illustrates examples of well positions in a well plate according to the illustration in FIG. 2C, into which the capture matrices are transferred after C. As each well contains only one capture matrix (or at least two capture matrices that have captured the biomolecules of interest from the same cell-laden matrix or two or more cell-laden matrices comprised in the same cultivation compartment), the required number of wells is n×m×k, with n being the column and m the rows of a cell culture device and k being the number of different time points at which the biomolecules of interest were captured. For example, if a microfabricated cell culture device contains 96 cell culture chambers and the secretion profiles are measured at 16 time points or 16 time intervals, an exemplary 1536 plate could be used for performing the capture matrix processing.

FIG. 3 schematically illustrates core elements of the sequenceable reaction product that is generated in step d):

A: Illustrates a schematic scaffold structure of the core elements. The sequenceable reaction product comprises:

(i) the barcode sequence (B_{S}) for indicating the specificity of the detection molecule (specificity information); and

(ii) a barcode sequence (B_{T}) for indicating a time information (e.g. time-point) in which certain biomolecules of interest have been secreted/detected (time information); and/or

(iii) a barcode sequence (B_{P}) for indicating a position information; and

(iv) optionally a unique molecular identifier (UMI) sequence, for quantifying the number of detection molecules that have bound a biomolecule of interest (information about quantity); and

(v) optionally an adapter sequence (AS) for sequencing.

As is also apparent from the illustrated embodiments, the order of the barcode sequences in the sequenceable reaction product may vary. Furthermore, additional sequence stretches (illustrated by white boxes) may or may not be present between the different barcode sequences/sequence elements.

B: The shown sequenceable reaction product can be obtained by the method depicted in FIG. 5. C: The shown sequenceable reaction product can be obtained by the method depicted in FIG. 6. D: The shown sequenceable reaction product can be obtained by the method depicted in FIG. 7. E: The shown sequenceable reaction product can be obtained by the method depicted in FIG. 8a. F: The shown sequenceable reaction product can be obtained by the method depicted in FIG. 9. G: The shown sequenceable reaction product can be obtained by the method depicted in FIG. 8b.

FIG. 4 illustrates that the present method allows to provide a pooled library of sequenceable reaction products that were obtained for different cell-laden matrices (position 1 and 2, wherein position/cell-laden matrix 1 is indicated by the barcode sequence B_{P1} and the position/cell-laden matrix 2 is indicated by the barcode sequence B_{P2}), different biomolecules of interest (antigen X and antigen Y, wherein the specificity for antigen X is indicated by the barcode sequence B_{S1} and the specificity for antigen Y is indicated by the barcode sequence B_{S2}) at two different time points (time-point 1 and time-point 2, wherein time-point 1 is indicated by the barcode sequence B_{T}, and time-point 2 is indicated by the barcode sequence B_{T2}). This concept can be extended for numerous additional positions, biomolecules of interest and time-points. In the illustrated embodiment, each sequenceable reaction product that originates from the barcode label of a single detection molecule comprises a unique UMI sequence (see UMI 1-8), thereby allowing to quantify the obtained information. The use of UMI sequences is known e.g. in the field or sequencing and therefore, does not need to be described in detail herein. As the information of sequenceable reaction products can be due to the comprised barcode sequences clearly assigned to the different positions, time points and biomolecules of interest, it is possible to pool all sequenceable reaction products into one library. The library can then be sequenced using current sequencing techniques such as NGS (Next-Generation-Sequencing), so that the information can be assessed by analyzing the sequencing results. The advantages compared to e.g. fluorescence based methods were described in detail above.

FIG. 5 illustrates an embodiment of the present invention, wherein the barcode label that is attached to the detection molecule (in the illustrated embodiment an antibody) comprises
a barcode sequence (B_{S}) for indicating the specificity of the detection molecule,
a barcode sequence (B_{T}) for indicating a time information, and
a unique molecular identifier (UMI) sequence.

The illustrated order of these sequence elements is not limiting and may accordingly differ (e.g. B_{T}, B_{S}, UMI or UMI, B_{S}, B_{T} etc.). The barcode label may be attached via a linker such as a photocleavable spacer. The sequence elements B_{S}, B_{T} and UMI are in the illustrated embodiment flanked by primer sequences (1) and (2) which provide target sequences for the amplification primers. The barcode label may be attached to the detection molecule prior to contacting the detection molecule with the capture matrix (“off-chip”). The detection molecule binds the captured biomolecule of interest to which it specifically binds as has been explained in conjunction with FIG. 2. The detection molecule may be added while the capture matrix is still in contact with the cell-laden matrix, or the capture matrix with the captured biomolecule(s) of interest can be separated from the cell-laden matrix prior to contacting the capture matrix with the detection molecules. The embodiment illustrated in FIG. 5 allows to reduce the number of processing steps. Thus, after incubating the capture matrix and the cell-laden matrix (or matrices) for release (e.g. secretion) and capturing of the biomolecules of interest and subsequent washing, the capture matrix may be contacted, e.g. perfused (e.g. if a microfabricated device as disclosed herein is used), with a solution containing the one or more types of detection molecules that are associated with a barcode label which already comprises as shown in the illustrated embodiment the specificity, the quantity and the time information. The capture matrix with the bound detection molecules may then be transferred to a collection position (e.g. a collection well of a 96-well device), for performing an amplification reaction. A primer or primer combination is added, as well as reagents required for performing the amplification reaction (e.g. polymerase, dNTPs, buffers). A barcode sequence (B_{P}) for indicating position information is introduced into the seqenceable amplification product via the primer or primer combination. In the illustrated embodiment, a primer combination in form of a primer pair is used, wherein the reverse primer hybridizes to the barcode label that is attached to the detection molecule, in the illustrated embodiment at primer sequence (2) of the barcode label. The forward primer is capable of hybridizing to the reverse strand of the barcode label that is generated when extending the reverse primer. In the illustrated embodiment the barcode sequence B_{P} is comprised in the forward primer. Alternatively, it could be comprised in the reverse primer. Furthermore, the forward and reverse primer preferably comprise adapter sequences AS at their 5′ ends as is illustrated in FIG. 5, which introduce into the sequenceable product adapter sequences for sequencing primers that are commonly used in sequencing platforms (S and P7 are shown as illustrative, non-limiting embodiments). FIG. 5 illustrates an embodiment wherein a primer pair is used. Alternatively, a single reverse primer could be used in a linear amplification reaction (e.g. by performing 2 to 20 or 5 to 15 extension cycles with the primer), thereby producing several copies of the reverse strand of the barcode label. In such embodiment, a reverse primer may be used which comprises the barcode sequence B_{P} and preferably, an adapter sequence AS at the 5′ end (e.g. S as shown in FIG. 5). To introduce a corresponding adapter sequence AS at the other end of the reverse strand, it is within the scope of the present disclosure to incorporate a matching adapter sequence AS in the 5′ region of the barcode label (i.e. 5′ of the sequence elements B_{S}, UMI and B_{P}), so that this information is incorporated into the reverse strand of the barcode label when the reverse primer is extended. In such embodiment, a primer sequence (1) is not required. Both embodiments (the use of a single primer and the use of a primer combination) allow providing a sequenceable reaction product as it is illustrated in FIG. 5C. As is apparent from the above description, the at least one oligonucleotide, optionally a primer, that is capable of hybridizing to the barcode label of the at least one type of detection molecule to which claim 1 refers corresponds in this embodiment to the primer that is used either alone or in the primer combination.

FIG. 6: shows a variation of the embodiment illustrated in FIG. 5. The barcode label attached to the detection molecule comprises in the illustrated embodiment
a barcode sequence (B_{S}), and
a unique molecular identifier (UMI) sequence.

Furthermore, it comprises primer sequences (1) and (2). The barcode label may again be attached to the detection molecule prior to contacting the capture matrix with the detection molecule. As explained above, the detection molecules bind the biomolecules of interest captured in the capture matrix. The capture matrix comprising the captured biomolecules of interest and the bound detection molecules comprising the barcode label indicating information about the specificity of the detection molecules (barcode B_{S}), as well as indicating a quantity information (here in form of an UMI sequence) may be in one embodiment transferred to a compartment (e.g. well) of a different device, also referred to herein as collection position (e.g. collection well), for performing an amplification reaction. Subsequently, an amplification may be performed using a primer or primer combination comprising
a barcode sequence B_{P} for indicating position information, and
a barcode sequence B_{T} for indicating time information.

A primer combination in form of a primer pair may be used for amplification, wherein the forward primer comprises the barcode sequence B_{P} and the reverse primer comprises the barcode sequence B_{T}, or vice versa. Accordingly, the barcode sequences for indicating a time information (B_{T}) and a position information (B_{P}) can be added within a collection position (e.g. well), i.e. after separating the capture matrix from the cell-laden matrix. The used primers may furthermore comprise adapter sequences AS at their 5′ ends as shown in FIG. 5. Furthermore, it is within the scope of the present invention that both barcode sequences B_{P} and B_{T} are comprised on a single primer (the forward or the reverse primer), and wherein the other primer merely comprises and thus introduces an additional adapter sequence into the sequenceable reaction product. Furthermore, as explained in conjunction with FIG. 5, a single primer may be used for performing several extension cycles, wherein said primer comprises the barcode sequences B_{T} and B_{P} and preferably, an adapter sequence AS (e.g. S as shown in FIG. 6). A corresponding adapter sequence at the opposite end of the sequenceable reaction product may be provided by incorporating a corresponding adapter sequence on the barcode label, positioned 5′ to the barcode sequence B_{S} and the UMI sequence. As explained above, primer sequence (1) is in such embodiment obsolete. All these embodiments allow providing a sequenceable reaction product as shown in FIG. 6C. As is apparent from the above description, the at least one oligonucleotide, optionally a primer, that is capable of hybridizing to the barcode label of the at least one type of detection molecule to which claim 1 refers corresponds in this embodiment to the primer that is used either alone or in the primer combination.

Incorporating the quantity information (UMI sequence) as part of the barcoded label that is associated with a detection molecule (as is illustrated in FIGS. 5 and 6) is advantageous because it renders obsolete an intermediate barcode label extension step wherein an adaptor barcode oligonucleotide capable of hybridizing to the barcode label is used as template in order to introduce an UMI sequence (as is illustrated e.g. in FIGS. 8 and 9). This is advantageous if it is desired to safe handling steps.

FIG. 7 to 9 illustrate various embodiments, wherein step d) comprises at least the following sub-steps

(aa) hybridizing at least one oligonucleotide to the barcode label of at least one type of detection molecule and extending said barcode label using the hybridized oligonucleotide as template thereby obtaining an extended barcode label attached to the detection molecule that additionally comprises sequence information of the hybridized oligonucleotide that was used as template, and

(bb) performing an amplification reaction with a primer or primer combination using the extended barcode label and/or the reverse complement thereof as template.

The at least one oligonucleotide that is capable of hybridizing to the barcode label of the at least one type of detection molecule to which claim 1 refers may correspond in these embodiments to the oligonucleotide (also referred to as adaptor barcode oligonucleotide) that is capable of hybridizing to the barcode label.

FIG. 7: The barcode label attached to the detection molecule comprises in the illustrated embodiment
a barcode sequence (B_{S}), and
a unique molecular identifier (UMI) sequence,
an adaptor sequence (1) at the 3′ end, and
preferably a primer sequence (1) in the 5′ region of the barcode label.

As explained above, the order of the barcode sequence B'S and the UMI sequence may vary. However, the adaptor sequence (1) is provided 3′ to these sequence elements. The barcode label, which preferably is provided by an oligonucleotide sequence that can be attached to the detection molecule via a photocleavable linker, can be attached to the detection molecule prior to contacting the capture matrix with the detection molecules. In the shown embodiment, step d) comprises a first substep (aa), wherein an adaptor barcode oligonucleotide is added, which is capable of hybridizing to the barcode label of the detection molecule. In the illustrated embodiment, the adaptor barcode oligonucleotide comprises an adaptor sequence (1)_{R} that is reverse complementary to an adapter sequence (1) of the barcode label of the detection molecule whereby it hybridizes to the barcode label. The adaptor barcode oligonucleotide may additionally comprise, as is illustrated in FIG. 7, a barcode sequence B_{T} and a primer sequence (2)_{R}, wherein the primer sequence (2)_{R} is located 5′ to the barcode sequence B_{T}. The adaptor barcode oligonucleotide comprising the time information B_{T} can be added to the compartment (e.g. of the microfabricated cell culture device) comprising the cell-laden matrix and the capture matrix comprising the captured biomolecules of interest. However, it is also within the scope of the present disclosure to remove the capture matrix with the captured biomolecules of interest prior to adding the adaptor barcode oligonucleotide (“off-chip”). The hybridized adaptor barcode oligonucleotide is used as template to provide an extended barcode label, which comprises the sequence information of the adaptor barcode oligonucleotide. Reagents necessary for performing an extension reaction (e.g. polymerase, dNTPs, buffers) are added and conditions provided to allow extension of the barcode label. The 3′ end of the adapter barcode oligonucleotide is in one embodiment extendable by the polymerase, whereby a double-stranded molecule is formed which comprises the reverse strand of the extended barcode label. Alternatively, the 3′ end of the adaptor barcode oligonucleotide is blocked so that it cannot be extended by the polymerase.

In this case, only a short double-stranded region is provided upon hybridization and extension of the barcode label, which comprises the adaptor barcode oligonucleotide and the corresponding extended region of the barcode label. The obtained extended barcode label comprises in the shown embodiment the following sequence elements: primer sequence (1), B_{S}, UMI, adapter sequence (1), B_{T} and primer sequence (2). The reverse complement of the extended barcode label, if provided upon extension, may be removed prior to the amplification step which is performed in step (bb) (see FIG. 7C), whereby a detection molecule is provided which comprises a single-stranded extended barcode label. Furthermore, an adaptor barcode oligonucleotide with a blocked 3′ end may be used which prevents that a reverse complement of the barcode label is formed at this stage. The oligonucleotide does not comprise sequences that would allow binding of the primer or primer combination that is used in amplification step (bb). Furthermore, the oligonucleotide can be easily removed from the amplification reaction, e.g. by purifying the amplification products using a size-selective purification method having a cut-off that removes the significantly shorter adaptor barcode oligonucleotides (and primers), while purifying the considerably longer amplification product (see FIG. 7D). After performing step d) (aa), an amplification reaction is performed in (bb). A primer or primer combination may be used that comprises a barcode sequence B_{P}, which thereby is introduced in the amplification product. Furthermore, the primer or primer combination may comprise an adapter sequence (AS). In the illustrated embodiment, a primer pair is used, wherein the forward primer comprises the barcode sequence B_{P} 5′ to the primer sequence that binds the reverse complement of the extended barcode label. This primer additionally comprises an adapter sequence (AS) (here: P7), 5′ to the barcode sequence B_{P} which thereby is introduced at one end of the amplification product. The reverse primer of said pair comprises in the illustrated embodiment a primer sequence (2)_{R} that is capable of hybridizing to the primer sequence (2) of the extended barcode label and which comprises an adapter sequence (AS) (here: S) at the 5′ end which thereby is introduced at the other end of the amplification product. Alternatively, the barcode sequence B_{P} may be provided in the reverse primer. If a single primer is used for amplification by performing several cycles of primer extensions, said primer then comprises the barcode label B_{P}. E.g. the reverse primer shown in FIG. 7 could be used, wherein the barcode label B_{P} is placed between the primer sequence (2)_{R} and the adapter sequence (AS) (here: S). To provide the obtained amplification product with a second adapter sequence (AS), such sequence may be already incorporated into the barcode label that is attached to the detection molecule and thereby, becomes incorporated into the amplification product. If only a single primer is used for amplification by performing several cycles of primer extension, it is not required to provide a primer sequence (1) in the barcode label. Instead, an adapter sequence AS for sequencing may be included instead of the primer sequence (1) as explained above. All these embodiments allow providing a sequenceable reaction product as it is illustrated in FIG. 7D. It follows from the above disclosure that the arrangement of the sequence elements B_{P}, B_{S}, UMI and B_{T} may vary depending on the used embodiment. E.g., the barcode sequence B_{P} may be located between the primer sequence (2) and the adapter sequence S, the order of the barcode sequence B_{S} and UMI sequence may be reversed and the primer sequence (1) may be missing, if only a single primer is used for amplification.

FIG. 8a: A variation of the embodiment shown in FIG. 7 is illustrated. In the shown advantageous embodiment, the barcode label comprises the barcode sequence B_{S} and the UMI sequence is introduced via the adaptor oligonucleotide sequence. The adaptor barcode oligonucleotide comprising the UMI sequence can be added to the compartment (e.g. of the microfabricated cell culture device) comprising the cell-laden matrix and the capture matrix, or the capture matrix with the captured biomolecules of interest is removed prior to adding the adaptor barcode oligonucleotide (“off-chip”). The UMI library size might be in the range of the binding capacity of a capture matrix. For example, if the capture matrix is capable of binding 1×10⁶ molecules, the UMI library might comprise 1×10⁶ different UMI sequences to ensure that each captured biomolecule of interest is labelled with a specific UMI. In contrast, if the UMI library is incorporated into the label barcode and a plurality of cell-laden matrices and corresponding capture matrices are processed in different compartments (multiplexing), the UMI library must comprise enough molecules to label more than one capture matrix. Thus, addition of the UMI library via the adaptor barcode oligonucleotide allows to reduce the UMI library size to the maximum binding capacity of the processed capture matrix. The reverse complement of the extended barcode label, if provided upon extension, may be removed prior to the amplification step which is performed in step (bb) (see FIG. 8a C). The removal of the reverse complement of the extended barcode label is beneficial to prevent processing of polymerase-extended products resulting from unspecific hybridizations. Thereby, a detection molecule is provided which comprises a single-stranded extended barcode label. Alternatively, an adaptor barcode oligonucleotide may be used that comprises a blocked 3′ end that cannot be extended by a polymerase so that a removal is not required. As explained in conjunction with FIG. 7, a polymerase extension reaction is performed using the adaptor barcode oligonucleotide as template, whereby an extended barcode label is provided which comprises the information of the oligonucleotide. The adaptor barcode oligonucleotide may again be extendable at its 3′ end, or the 3′ end may be blocked to prevent extension by the polymerase. After substep aa) of step d), an amplification step is performed in (bb), wherein a primer or primer combination is used, which comprises the barcode sequence B_{T} and the barcode sequence B_{P}. The amplification reaction is preferably performed in a compartment that does not comprise the cell-laden matrix. Various transfer options for the detection matrix are described elsewhere herein. For the shown embodiment it is preferred, that the one or more capture matrices are removed from the proximity of the cell-laden matrix and transferred into a compartment (e.g. well) prior to adding the detection molecules for binding (see FIG. 2C). If a primer pair is used as is illustrated in FIG. 8a, the barcode sequence B_{T} may be located on the reverse primer and barcode sequence B_{P} may be located on the forward primer, or vice versa. Furthermore, the primers may comprise adapter sequences (AS) as shown in FIG. 8a (see “S” and “P7). Both barcode sequences B_{P} and B_{T} may also be provided on a single primer (forward or reverse), wherein said primer preferably comprises an adapter sequence (AS) for sequencing at the 5′ end. The second primer may then serve the purpose to support the amplification and to introduce a second adapter sequence (AS) for sequencing at the opposite end. Additionally, it is again possible to use a single reverse primer which hybridizes to the extended barcode label and which comprises the barcode sequences B_{T} and B_{P}, preferably in addition to an adapter sequence at its 5′ end. A second adapter sequence may be provided in the 5′ region of the barcode label that is attached to the detection molecule so that it is incorporated also in the obtained amplification product. As noted above, a primer sequence (1) in the barcode label is not required if a single primer is used to perform several primer extension cycles for amplification and the primer sequence (1) could be replaced by an adapter sequence AS. All these embodiments allow providing a sequenceable reaction product as is illustrated in FIG. 8a D. It again follows from the above disclosure that the arrangement/order of the sequence elements B_{P}, B_{S}, UMI and B_{T} may vary depending on the used embodiment.

FIG. 8b: A variation of the embodiment shown in FIG. 7 is illustrated. In the shown embodiment, the barcode label comprises the barcode sequence B_{S} and the UMI sequence as well as the barcode sequence B_{P} are introduced via the adaptor oligonucleotide sequence. The adaptor barcode oligonucleotide comprising the UMI sequence and the barcode sequence B_{P} can be added to the compartment (e.g. of the microfabricated cell culture device or the collection well) comprising the cell-laden matrix and the capture matrix, or the capture matrix with the captured biomolecules of interest is removed prior to adding the adaptor barcode oligonucleotide as is illustrated in FIG. 8b (“off-chip”). The illustrated embodiment, wherein the adaptor barcode oligonucleotide comprising the barcode sequence B_{P} and an UMI sequence is added after removal of the capture matrix (see FIG. 2C), is advantageous. This reduces the required UMI library size due to combination of UMIs with the barcode B_{P}. The same UMIs can be used for different compartments/collection wells, which are clearly distinguishable and identifiable based on the barcode B_{P}. The reverse complement of the extended barcode label, if provided upon extension due to elongation of the adaptor barcode oligonucleotide, may be removed prior to the amplification step which is performed in step (bb) (see FIG. 8b C). The removal of the reverse complement of the extended barcode label is beneficial to reduce the number of false hybridized elongation products. As explained in conjunction with FIG. 7, a polymerase extension reaction is performed using the adaptor barcode oligonucleotide as template, whereby an extended barcode label is provided which comprises the information of the oligonucleotide. The adaptor barcode oligonucleotide may again be extendable at its 3′ end, or the 3′ end may be blocked to prevent extension by the polymerase. After step aa), an amplification step is performed in (bb), wherein a primer or primer combination is used, which comprises the barcode sequence B_{T}. The amplification reaction is preferably performed in a compartment that does not comprise the cell-laden matrix (transfer options are described herein). If a primer pair is used as illustrated in FIG. 8b, the barcode sequence B_{T} may be located on the forward primer, or alternatively on the reverse primer. Furthermore, the primers may comprise adapter sequences (AS) as shown in FIG. 8b (see “S” and “P7). Additionally, it is again possible to use a single reverse primer which hybridizes to the extended barcode label and which comprises the barcode sequences B_{T}, preferably in addition to an adapter sequence at its 5′ end. A second adapter sequence may be provided in the 5′ region of the barcode label that is attached to the detection molecule so that it is incorporated also in the obtained amplification product. As noted above, a primer sequence (1) in the barcode label is not required if a single primer is used to perform several primer extension cycles for amplification and the primer sequence (1) could be replaced by an adapter sequence AS. All these embodiments allow providing a sequenceable reaction product as it is illustrated in FIG. 8b D. It again follows from the above disclosure that the arrangement/order of the sequence elements B_{P}, B_{S}, UMI and B_{T} may vary depending on the used embodiment.

FIG. 9: A further variation of the embodiment shown in FIG. 7 is illustrated. The barcode label only comprises the barcode sequence B_{S} while the UMI sequence and the barcode sequence B_{T} are introduced via the adaptor oligonucleotide sequence. These two sequence elements are flanked 3′ by the adaptor sequence (1)_{R} and 5′ by the primer sequence (2)_{R}. The order of B_{T} and UMI may be reversed in the adaptor barcode oligonucleotide. The adaptor barcode oligonucleotide comprising the UMI sequence and the barcode sequence B_{T} can be added to the compartment (e.g. of the microfabricated cell culture device) comprising the cell-laden matrix and the capture matrix (“on-chip”, i.e. time and quantity information (UMI) may in this embodiment added within a microfabricated compartment containing the capture matrix and the cell-laden hydrogel matrix), or the capture matrix with the captured biomolecules of interest may be removed prior to adding the adaptor barcode oligonucleotide (“off-chip”). As explained in conjunction with FIG. 7, a polymerase extension reaction is performed using the adaptor barcode oligonucleotide as template, whereby an extended barcode label is provided which comprises the sequence information of the oligonucleotide. The adaptor barcode oligonucleotide may again be extendable at its 3′ end, or the 3′ end may be blocked to prevent extension by the polymerase.

After step aa), an amplification step is performed in (bb), wherein a primer or primer combination is used, which comprises the barcode sequence B_{P}. The amplification reaction is preferably performed in a compartment that does not comprise the cell-laden matrix. If a primer pair is used as illustrated in FIG. 9, the barcode sequence B_{P} may be located on the forward primer, or alternatively on the reverse primer. Furthermore, the primers may comprise adapter sequences (AS) as shown in FIG. 9 (see “S” and “P7). Additionally, it is again possible to use a single reverse primer which hybridizes to the extended barcode label and which comprises the barcode sequences B_{P}, preferably in addition to an adapter sequence at its 5′ end. A second adapter sequence may be provided in the 5′ region of the barcode label that is attached to the detection molecule so that it is incorporated also in the obtained amplification product. As noted above, a primer sequence (1) in the barcode label is not required if a single primer is used to perform several primer extension cycles for amplification and the primer sequence (1) could be replaced by an adapter sequence AS. All these embodiments allow providing a sequenceable reaction product as it is illustrated in FIG. 9D. It again follows from the above disclosure that the arrangement/order of the sequence elements B_{P}, B_{S}, UMI and B_{T} may vary depending on the used embodiment.

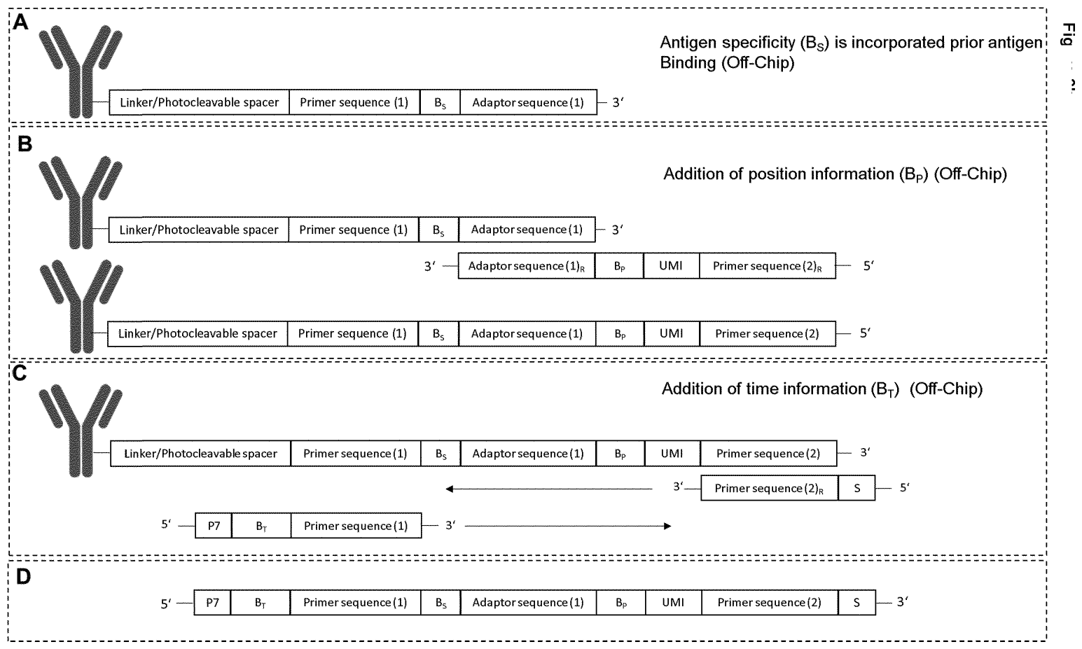

FIG. 10 is an illustration of a particle trap 17 for encapsulation of a single particle, here a single-cell. The trap 17 is located above a microfabricated elastomer valve portion 14.
FIG. 10A: The top microfabricated layer 23 is first perfused with a particle suspension 36, i.e. here a cell suspension. Single cells 20 are trapped and immobilized in the hydrodynamic trap 17 located above a microfabricated valve portion 14. Subsequent opening of the microfabricated valve portion 14 results in a fluid flow from the top layer 23/second channel 12 into the bottom layer 21/first channel 11 that is filled with an immiscible (with the respect to the fluid within the second channel) second fluid 37, in particular an oily fluid. The trapped cell 20 is thereby transferred into the formed droplet 31, wherein the fluid of the cell suspension 36 surrounds the captured cell 20. The fluid of the cell suspension 36 and the particle constitutes a droplet 31.
FIG. 10B: is an illustration of the particle trap 17 of FIG. 10A in top view. The generic single particle trap 17 is located above/adjacent to the microfabricated elastomer valve portion 14. The trap 17 comprises a bottleneck section 16, which fluid opening is smaller than the particle 20 to be trapped. A first particle (cell) arriving at the trap is captured by the trap. All further particles (cells) arriving subsequently at the trap take the way along a bypass section 18. 38 illustrates an optional impedance measuring device, 39 illustrates an optional radio frequency application device.
FIG. 10C: is an illustration of an amended trap group for the immobilization of two particles 20, in particular cells, located in two separate neighboring traps 17n above the microfabricated valve portion 14. Opening of the valve portion 14 may result in a co-encapsulation of two trapped cells 20 into one droplet 31, because the valve portion 14 leads from both traps 17n into the same first channel 11 below both traps 17n. Using this embodiment, two different cells 20 can be encapsulated within one single droplet 31.
FIG. 10D shows a trap group in schematic view. Each of the neighboring traps 17n is loaded from a separate channel 12′, 12″, in which the same pressure p2 is applied to the fluid, to achieve droplets of the same size. At first the traps 17n are loaded; when all traps 17n are loaded a washing fluid can be applied to clean the trapped cells. Subsequently the valve portions 14 are opened to include the cells 20 through one valve section 14 simultaneously into one droplet 31. A plurality of such trap groups having two neighboring traps 17n can be arranged in one test device.

FIG. 11 is an illustration of hydrodynamic resistances of a microfabricated geometry for the controlled removal and transfer of particles such as capture matrices and/or cell-laden hydrogel matrices to an exit portion. In addition, that microfabricated geometry can be arranged within an array enabling the positioning and removal of hundreds to thousands of particles. Said microfabricated geometry comprises the hydrodynamic resistances R0, R1, R2, R3, R4 within one compartment 32, here at the example of compartment 32m2n2 in position of column m2 and row n2. R0 indicates the hydrodynamic resistances at a matrix trap 33, R1-R4 indicate the hydrodynamic resistances of different paths within compartment 32, with R1, R4>R2, R3. P1 indicates an entrance of a main fluid flowing through the compartment 32 to an exit indicated by P2. The main feeding channel 41 optional here.
FIG. 11A: During normal operation the main fluid stream moves from top to down (first direction of flow S1 along first path of flow 51 or optional along main feeding channel 41), since the stream takes the “easier way” through smaller resistances R2, R3. Merely a negligible part of the fluid flows through path of resistances R1, R4. Here all triggering commands Cm2, Cn2 are set to zero.
FIG. 11B: By triggering a valve Vm2 by command Cm2=1 in the path of R2, resistance R2 of this path will significantly increase. The main fluid now moves from P1 to P2 via paths of resistances R4 and R3 along third path of flow 53. The flow at R0 is now stopped, but not reversed.
FIG. 11C: By triggering a valve Vn2 in the path of R3 command Cn2=1, resistance R3 of this path will significantly increase. The main fluid now moves from P1 to P2 via paths of resistances R2 and R1 along fourth path of flow 54. The flow at R0 is now stopped, but not reversed.
FIG. 11D: Only when both the resistances in paths of R2 and R3 is increased, by triggering the valves Vm2 and Vn2 by commands Cm2 and Cn2 set to 1, the flow at position R0 within the matrix trap 33 is reversed. The main fluid now moves from P1 to P2 via paths of resistances R4, R0 and R1 along fourth path of flow 54. A matrix 31 that is located within the matrix trap 33 at R0 is subsequently removed from the trap position. The group of the both valves Vm2, Vn2 is here called at the valve arrangement 40m2n2 of the observation chamber 32m2n2 exemplary.

The presented microfabricated geometry can be e.g. used to accomplish the disclosed methods. In particular, the microfabricated geometry can be used to position for example one-cell-laden hydrogel matrix and a capture matrix in proximity within one compartment. In addition, said microfabricated geometry enables the removal of the capture matrix while the cell-laden matrix remains within its position. One advantage of the presented microfabricated geometry is its compatibility with an array arrangement. Thus, multiple microfabricated geometries can be connected to generate an addressable n×m array containing at least one cell-laden matrix and a capture matrix at each position (n|m) of said array and while still being capable of transferring capture and/or cell-laden matrices located at a defined position. Matrices such as capture matrices or cell-laden matrices can be delivered to the microfabricated geometry within a droplet that is located within a fluid that is immiscible with an aqueous fluid. Said fluid can be an oil such as fluorinated oil (e.g. HFE-7500). If matrices are provided within a droplet, the matrix formation may not have been started, may be ongoing or may be finished (droplet contains a fully polymerized/gelled matrix). In addition, fully polymerized/gelled matrices located within an aqueous phase may be delivered to the microfabricated geometry. For example, capture matrices may be formed prior to the addition to the cell culture device enabling a detailed quality control of the capture matrices using various characterization methods.

FIG. 12 shows simulations with a generic microfabricated cell culture device for trapping matrices, in particular spherical hydrogel matrices (e.g. cell-laden matrix, capture matrix), in a specific location 32, which is also described in more with reference to the circuit diagram of FIG. 11.
FIGS. 11A and 12A: Normal operation. No microfabricated valves are closed; consequently resistances R2 and R3 in fluid lines 502 and 503 are much smaller than resistances R1 and R4 in fluid lines 501 and 504. The fluid flow perfuses the trap geometry 33 from top to bottom in direction S1. Thus, a particle (cell) is immobilized within the trapping structure 33.
FIGS. 11B and 12B: The bottom left microfabricated valve represented by resistance R3 is closed. The main fluid stream goes through the upper channel.
FIGS. 11C and 12C: The main fluid stream goes through the bottom channel. A particle is pushed into the trap.
FIGS. 11D and 12D: Only when both microfabricated valves represented by resistances R2 and R3 are closed the reverse fluid flow in direction S2 removes that particle from the trapping structure 33.

In terms of the current disclosure, the generic trapping structure 33 is adapted to position at least two particles such as a cell-laden matrix and a capture matrix. A detailed description of such a positioner is given in FIG. 13 to 17. The generic microfabricated cell culture can be operated in several states including the perfusion with fluid direction S1 and the perfusion in fluid direction S2. The perfusion in fluid direction S1 enables the efficient washing of capture matrices after analyte binding and subsequent washing with detection molecules. In addition, the generic microfabricated compartments can be closed thereby enabling the generation of a closed reaction compartment having a defined reaction volume. This is critical, as secreted analytes have to remain within the same reaction compartment as the capture matrix to allow the binding of analytes to the capture molecules. The perfusion in fluid direction S1 enables the removal a capture matrices, for example after the detection molecules were added and subsequent transfer into another device. The controlled transfer of capture matrices to a pre-defined position of another device is a crucial step as the capture matrix can be further processed without loosing the information, that the capture matrix was positioned close to the cell-laden matrix at position (n1m). This information is required for performing a unique assignment of the data generated by analysing the capture matrix (secretion profiled) to the corresponding cell(s) that have secreted the analysed molecules.

FIG. 13 to 15 illustrates embodiments for removing a matrix, e.g. a capture matrix, by reverse flow cherry picking (RFCP):

FIG. 13A shows two matrices located within close proximity, e.g. a capture matrix (31C) and a cell-laden matrix (31A). A reverse flow results in a force F2 acting on capture matrix 2 (31C) and in a force F1 acting on cell-laden matrix 1 (31A) with F2 being larger than F1. Thus, at a certain flow rate only capture matrix 2 (31C) is removed, while the cell-laden matrix 1 (31A) remains in the compartment. FIG. 13B shows corresponding hydrodynamic resistances for generating two different forces acting on said matrices.

FIG. 14 shows the removal of particles in particular matrices (31C, 31A) located within a positioning mean provided in frame of a RFCP mechanism by using different reverse flow rates. An increase of the reverse flow rate might result in a removal of a first capture matrix 31C while all matrices located within different (microfabricated) compartments might remain within their position. A further increase of the flow rate might result in a removal of a second matrix from the same compartment (e.g. the cell-laden matrix 31A) without removing matrices located within other compartments. This is advantageous, as capture matrices can be first transferred for subsequent processing and analysis. Afterwards, the cell-laden matrix may be collected as well for further characterization of said cell(s) using established methods. In particular, the collected cell(s) may be characterized in terms of their genotype (e.g. by using RT-PCR or (single cell) RNA-seq) enabling the assignment of genotypic information to phenotypic information (such as the generated secretion profile of the one or more biomolecules of interest as presented in the current disclosure).

FIG. 15 shows the sequential removal of three particles in particular matrices or droplets (droplets 31-A-C) by RFCP which have been positioned in proximity by using a positioner. In one embodiment, matrix 31A may be a cell-laden matrix containing cell(s) of type 1, matrix 31B may be a cell-laden matrix containing cell(s) of type 2, whereas cell(s) of type 1 and type 2 may be the same or different, and matrix 31C may be a capture matrix. A reverse flow results in a force F3 acting on a matrix 31C, in a force F2 acting on matrix (matrix 31C) and in a force F1 acting on matrix 31A with F3 being larger than F2 being larger than F1. Thus, at a certain flow rate only matrix (matrix 31C) is removed. Increasing the reverse flow rate leads to the sequential removal of the other matrices. B) Corresponding hydrodynamic resistances for generating three different forces acting on said matrices. An exemplary embodiment is also shown in FIG. 16. The removal of the capture matrix (31C) without removing the matrices 31A and 31B enables the repeated positioning of a “fresh” capture matrix having no analytes bound to the capture molecules thereby allowing the detection of analytes secreted within different time intervals. In one embodiment, the matrices 31A and 31B as well as the capture matrix 31C are located within a droplet that is located within a fluid immiscible with an aqueous fluid. Within the positioner the droplets containing the matrices 31A-C interface with each other. In particular, the droplets containing the matrices 31A-C merge with each other forming one droplet containing the matrices 31A-C thereby reducing the reaction volume to the volume of approximately the three matrices 31A-C which increases the analyte concentration and thus the sensitivity. This may also be done with two droplets/matrices, one cell-laden matrix and one capture matrix. In addition, the matrix 31C may be removed from the common droplet and a droplet fission may be performed using the RFCP mechanism. A fresh droplet containing a capture matrix may be delivered to the remaining droplet containing matrix 31A and 31B. In another advantageous embodiment, the matrices 31A and 31B may be incubated within one common droplet first. Afterwards, a capture matrix located within a droplet may be positioned and merged with the common droplet containing matrices 31A and 31B. This has the advantage, that secreted molecules can first accumulate within the common droplet thereby enabling paracrine and autocrine signalling and subsequently be captured by addition of the capture matrix. The same procedure may be performed using a positioner for positioning of only two particles instead of three.

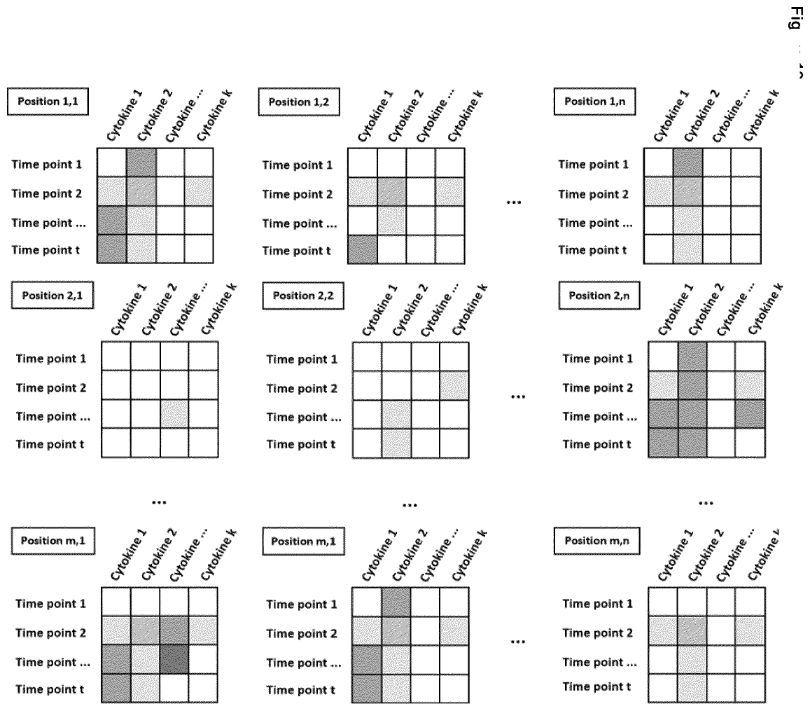

FIG. 16: FIG. 16B shows a generic location, details of which are shown in FIG. 16A. The location comprises two bypass sections 35 circumventing a group of positioner 33. Here three bottleneck sections 34A, 34B, 34C are provided in sequence each defining a positioner 33A, 33B, 33C. During loading of the location a first particle in particular a droplet/matrix arriving at the positioners 33 will move up to the first positioner 33A and will be retained in the first positioner 33A. A second droplet/matrix arriving subsequently will move up to the second positioner 33B upstream of the first positioner 33A and will be retained in the second positioner 33B. A third droplet/matrix arriving subsequently will move up to a third positioner 33C upstream of the second positioner 33B and will be retained in the third positioner 33C. It is possible to provide any number of bottleneck sections 34/positioners 33 to enable a row of droplets/matrices 31 of a predetermined number. When all the positioners are occupied further droplets/matrices will follow the bypass section 35 and approach the locations at a downstream position along first fluid direction S1. When the fluid is reversed to untrap the droplets/matrices at first droplet/matrix upstream (when viewed in first fluid direction S1) in third bottleneck section 34C will be untrapped. Due to the hydraulic design in the droplet/matrix trap the droplets/matrices retained in the upstream positioner 33C will be subject of an increased hydraulic pressure compared to the droplets/matrices retained in the downstream positioner 33A, 33B. Thus, upon reversal of the fluid direction into the second fluid direction S2 at first the droplet/matrix in the most upstream positioner 33C will be untrapped and can be delivered to an exit section e.g. at P2 (see FIG. 21). At second the fluid pressure between P1 and P2 will be increased, so that subsequently also the droplets/matrices retained in the more downstream positioner 33A, 33B will be untrapped and will also be delivered to exit at P2. A suitable hydraulic design can be obtained by CFD simulations.

FIG. 17 shows a sequential removal of three matrices in a trap having 3 bottleneck sections each by a first (downstream) matrix 31A, second matrix 31B and third (upstream) matrix 31C, without affecting matrices located within other compartments (for example from a cell culture device, preferably a microfabricated cell culture device).
During a first untrapping period I low pressure or flow rate p1 is applied through fluid, so that all matrices remain trapped.
During a second period an increased pressure or flow rate p2 is applied through the fluid, which is strong enough to remove merely upstream matrix 31C; the other matrices 31B, 31A remain trapped.
During a third period Ill a further increased pressure or flow rate p3 is applied through the fluid, which is strong enough to remove second matrix 311B; the downstream matrix 31A remains trapped.
During a fourth period IV a further increased pressure or flow rate p4 is applied through the fluid, which is strong enough to remove third upstream matrix 31A. The pressure can be applied through input P1 (see FIG. 6).

FIGS. 13 and 14 show the same concept as described with reference to FIGS. 15 and 16, but merely for the use of two matrices 31A, 31C to be retained within one matrix trap, having two bottleneck sections 34A, 34C.

FIG. 18 is an illustration of a workflow for generating a time-lapse profile of one or more biomolecules of interest. To this end, at least two matrices (a cell-laden matrix 31A, and a capture matrix 31B) are positioned in a first step within a trap (33A, 33B) located within a compartment (32). This may be a trap for selective removal of trapped matrices as an exemplary embodiment is also shown in FIGS. 13 and 14. The cell-laden matrix (31A) contains at least one cell (20). In addition, said cell-laden matrix (31A) may be held stationary for a defined period. A capture matrix (31B) is positioned next to the cell-laden matrix (31A). The capture matrix may contain one or more types of capture molecules for capturing one or more biomolecules of interest that are secreted by the cell (20) provided in the cell-laden matrix. In a particular embodiment, the fluid surrounding the trapped matrices might be replaced by an oily fluid in a next step. Thus, the reaction volume is decreased to approximately the volume of both matrices (31A,31B). This has the advantage, that the reaction volume is fixed to a defined volume and the concentration of secreted biomolecules of interest is increased thereby increasing the measurement sensitivity of a potential detection mechanism. In a next step, both matrices (31A, 31B) may be held stationary for a defined period in which secreted biomolecules of interest might be released from the cell and diffuse to the capture matrix 31B where they are bound by the provided capture molecules. Afterwards, the fluid surrounding said matrices might be exchanged again enabling washing of trapped matrices and adding a detection molecule that is labelled with a barcode label as is described herein. The capture matrix (31B) is then removed by applying a reverse flow as disclosed and may be collected in a compartment of another device, e.g. the well (“collection position”) of another format, such a well plate while the cell-laden matrix 31A is held stationary. Afterwards, a new second (capture) matrix (31B) is provided and positioned again in 33B and the process is repeated. This method has the advantage, that secreted biomolecules of interest can be captured in a time-lapse manner and analysed either within the compartment (32) or after collection of said matrices (31B) in a different device. Secreted molecules may be cytokines, growth factors and the like.

FIG. 19 is an illustration of data that might be generated using the described time-lapse cytokine profiling technique. The method according to the present invention which uses different barcode sequences B_{S}, B_{T} and/or B_{P} that are provided within a sequenceable reaction product, that moreover can be pooled as is described herein has the advantage that the data can be generated in a time and cost efficient manner using sequences approaches. In addition, said data may be coupled to additional information about cell(s) located within the cell-laden matrices (e.g. phenotypic data gained with methods such as immunostaining, genotypic data gained with methods such as RT-PCR, RNA-seq).

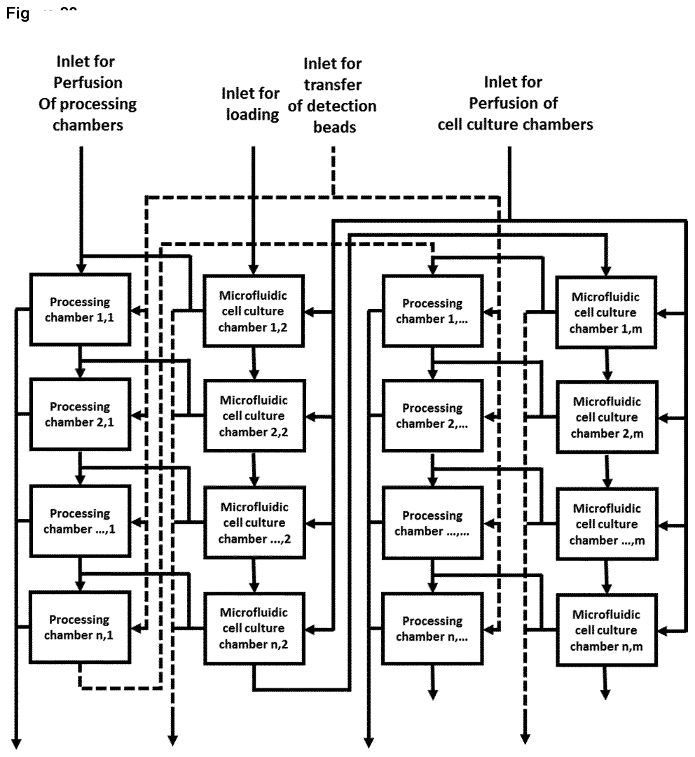

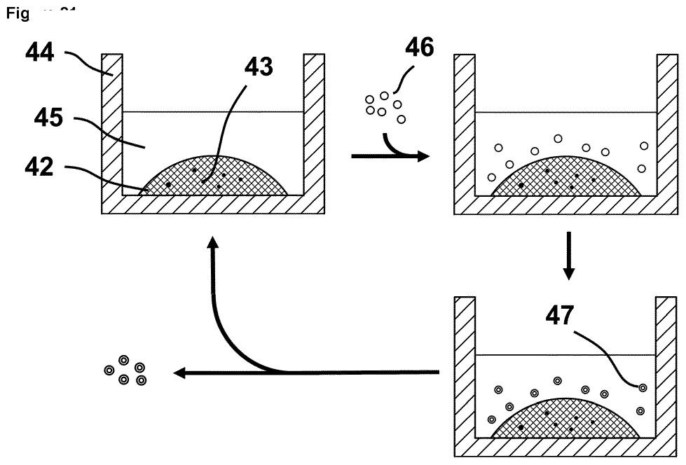

It is furthermore referred to following Figures of PCT/EP2018/074526, which are including the corresponding figure description herein incorporated by reference:
FIG. 2, showing a microfluidic array. Said array may be used for cultivating and analyzing hundreds to thousands of cell-laden matrices with the disclosed methods and for handling (positioning and transfer/removal) of the corresponding capture matrices.
FIGS. 22, A, B and C showing an array of the compartments controlled by RFCP.
FIG. 31 shows a workflow for the on-demand multi step stimulation of cells in a matrix.
FIG. 35 shows schematically an embodiment of a microfabricated geometry for immobilizing a matrix. Said microfabricated geometry can be adjusted for the immobilization

FIG. 20 is an illustration of the structure of a microfabricated cell culture device that enables the processing of capture matrices (e.g. contacting with detection molecules) located within another compartment than the compartment containing cell-laden matrices. This has the advantage, that the capture matrix processing can be separated from the cultivation of cells. Thereby, cell(s) located within cell-laden hydrogel matrices are not affected by the processing of the capture matrix. In addition, analytes secreted during the processing of a capture matrix comprising bound analytes from a prior incubation period, can be captured again using a “fresh” capture matrix that initially (during delivery) has no analytes bound thereby reducing the loss of any molecules secreted during the processing of the capture matrix. To enable the separation of the capture matrix processing from the cultivation process, a further compartment for the processing of the capture matrix (processing chamber) is added to the device. The compartment containing the one or more cell-laden matrices (microfluidic cell culture compartment) is connected to a processing chamber for receiving at least one capture matrix. Both compartments (the processing chamber for the at least one capture matrix as well as the corresponding microfluidic cell culture chamber for the at least one cell-laden matrix) may be structured as illustrated in FIGS. 11-17. The separation of the two processes may be done by connection the exit portion p2 of a microfluidic cell culture chamber with the feeding line 41 of a processing chamber. In addition, the exit portion of a microfluidic cell culture chamber may be connected to the exit portion (common exit portion) of at least one second microfluidic chamber. At least one valve may be used to switch between the feeding line of a processing chamber and the common exit portion. Thus, a matrix that is removed from the microfluidic cell culture chamber may be either directed towards a processing chamber or to the common exit portion. For example, a capture matrix located within microfluidic cell culture chamber 1,2 may be directed to the feeding line of processing chamber 1,1 and the capture matrix may be positioned within said processing chamber. A cell-laden matrix may be directed towards the common exit portion for direct collection without any processing. The exit portion p2 of a processing chamber is connected to the exit portion of at least one second processing chamber. Thus, after the processing of a capture matrix is done, the capture matrix can be removed from the processing chamber and be transferred into another format of a device. In addition, the processing chambers are connected in series and can be perfused without perfusing the microfluidic cell culture chambers. This can be done as all processing chambers share a common feeding line. In addition, all microfluidic cell culture chambers share a common feeding line that is different than the feeding line for the processing chambers. The processing chambers and the microfluidic cell culture chambers may be arranged in an addressable n×m array in which the flow at a position n|m can be reversed by providing a common group command as shown in FIG. 11.

FIG. 21 illustrates an embodiment, wherein the cell-laden matrix is incubated in a compartment of a cell culture plate. The cell-laden matrix (42) comprising at least one cell (43; here multiple cells) is provided in a compartment (44), e.g. a well, of a cell culture plate. FIG. 21 only shows the compartment (well) of such cell culture plate. The cell-laden matrix (42) is covered by a liquid (45), e.g. cell culture media. After incubating the cell-laden matrix (42), at least one capture matrix (46) is added to the compartment (44) which contains the liquid (45) and the cell-laden matrix (42). The capture matrix is provided in the illustrated embodiment by a plurality of capture beads. The added capture matrix (46) comprises one or more types of capture molecules which allow binding of the one or more biomolecules of interest (47) as disclosed herein. After binding of the released biomolecule(s) of interest to the capture matrix (in the shown embodiment a plurality of capture beads), the capture matrix with the bound biomolecules of interest (47) can be transferred, e.g. to another position, such as a different compartment (e.g. different well of a cell culture plate). Then, one or more further cycles of incubation of the cell-laden matrix (43) and capture matrix (46) addition can be performed (see arrow). Afterwards, steps c) and d) and optionally step e) of the method according to the present disclosure are performed (not shown in Figure).

### EXAMPLES

In the following examples, materials and methods of the present invention are provided. It should be understood that these examples are for illustrative purposes only and are not to be construed as limiting this invention in any manner.

### I. General Method Steps

Cell Encapsulation and Matrix Positioning

A single cell or multiple cells are encapsulated within a matrix to provide a cell-laden matrix. The matrix material is preferably provided by a hydrogel. Encapsulation into the matrix might be done using techniques such as droplet formation using flow focusing geometries or droplet on demand systems with corresponding sorting mechanisms, subsequent hydrogel formation and demulsification of cell-laden hydrogel matrices located within droplets. A suitable encapsulation method for a particle, here at least one cell, is described in detail in PCT/EP2018/074526, herein incorporated by reference. The described methods inter alia allow to center the cell within a hydrogel bead, thereby providing a cell-laden matrix. Droplet and hydrogel matrix size may be selected in embodiments from a range of 1 μm to 1000 μm, preferably 5 μm to 500 μm, more preferably 30 μm to 200 μm. Suitable ranges were also described elsewhere herein.

The provided cell-laden matrix is then positioned within a compartment of the cell culture device. The compartment of the cell culture device comprising the cell-laden matrix may have one or more of the following characteristics:
i. it can be selectively opened and closed using microfabricated valves, such as Quake Valves or vertical membrane valves as described in PCT/US2000/017740 or preferably PCT/EP2018/074526, respectively;
ii. it comprises a positioning mean which can be a microfabricated geometry for positioning or immobilizing matrices (e.g. cell-laden matrix and/or capture matrix); and/or
iii. it comprises a microfabricated geometry for removing one or more matrices while one or more other matrices remain within their position. This might be achieved by a valve arrangement adapted to provide fluid passing through a positioning mean (e.g. RFCP geometry as discussed above and as disclosed in PCT/EP2018/074526).

A capture matrix comprising capture molecules (e.g. immobilized antibodies) having a specificity against a defined biomolecule of interest (e.g. target analytes such as cytokines, chemokines, TNF or interleukins) is provided and positioned next to or in close proximity to the cell-laden matrix (e.g. preferably within the same compartment of a cell culture device, preferably a microfabricated compartment of a cell culture device) so that biomolecules of interest that are released, e.g. secreted, from the at least one cell may diffuse towards the capture matrix so that the capture molecules of the capture matrix can bind and thus capture the biomolecules of interest. Each microfabricated compartment contains a pre-defined number of matrices. In a particular embodiment, a microfabricated compartment comprises exactly one cell-laden matrix and one capture matrix. The distance between the capture matrix and the cell-laden matrix might be between 0 μm (the hydrogel matrices are in direct contact) to 100 μm or more. The positioning of a pre-defined number of different matrices might be achieved using a position mean such as a hydrodynamic trapping structure, preferably a microfabricated geometry for matrix immobilization (as disclosed herein and in PCT/EP2018/074526, herein incorporated by reference).

Generation of Compartments

An isolated compartment with a defined volume may be created by selectively closing/isolating the compartment e.g. by actuating corresponding microfabricated valves and/or exchanging the first fluid (e.g. aqueous phase) against a second fluid (which may be a phase immiscible with water, such as an oil phase, preferably a fluorinated oil; also referred to as biphasic compartment generation as described in PCT/EP2018/074526 and PCT/EP2018/074527), whereby the reaction volume is reduced. At this step the capture matrix and the cell-laden matrix are located within the same hydrophilic reaction volume. The reaction volume may be closed by valve actuation, whereby an isolated compartment is generated. In a particular embodiment, the isolated and closed microfabricated compartment has a volume in the range of 1 nL to 500 nL, preferably 10 nL to 50 nL.

In another advantageous embodiment, the reaction volume can be further reduced by using an alternating biphasic compartment generation described in the present disclosure. Thus, in one embodiment the aqueous phase surrounding the positioned hydrogel matrices might be exchanged by an immiscible fluid such as a fluorinated oil (e.g. HFE-7500) thereby reducing the volume compartment to a volume that approximately corresponds to the volume of the trapped matrices. In one embodiment, the reduced volume of the aqueous phase containing the hydrogel matrices might be in the range of 0.05 nL to 10 nL, preferably 0.4 nL to 0.6 nL.

Incubation of the Cell-Laden Matrix

The cell-laden matrix is incubated for a defined time period (e.g. 1 h, 2 h or more). The cell-laden matrix within the compartment is provided in a surrounding/under conditions so that cell(s) located within the matrix may release, e.g. secrete, one or more biomolecules of interest. The biomolecules of interest diffuse to the neighboring capture matrix where they are bound by the immobilized capture molecules having the corresponding specificity towards the released biomolecule of interest. In case an alternating biphasic compartment was generated, the biomolecules of interest may remain within the aqueous phase comprised in the cell-laden matrix, wherein the provided a capture matrix also comprises an aqueous phase, which can become available for diffusion of biomolecules of interest.

Intermediate Steps Such as Washing Steps

Washing steps may be performed at any time point throughout the method according to the present disclosure.

The fixed matrices may optionally be washed with a washing buffer such as PBS to remove unbound biomolecules of interest by perfusing the compartment of the cell culture device.

According to one embodiment, the isolated compartment is again opened (e.g. by actuating a microfabricated valve). If an aqueous phase surrounding the matrices has been replaced by an oil phase (e.g. HFE-7500), the oil phase may again be replaced by an aqueous phase. This is done by perfusing the microfabricated system with an aqueous phase such as PBS. This procedure is very efficient, as the same buffer can be perfused through all compartments.

Adding One or More Types of Detection Molecules

The capture matrix is then contacted with one or more types of detection molecules. The compartment comprising the capture matrix may be in one embodiment perfused with a solution containing one or more types of detection molecules (e.g. with an adjustable concentration) for a defined time period. The detection molecules bind to their captured biomolecules of interest. The detection molecules are associated with a barcode label which comprises at least a barcode sequence (Bₛ) indicating the biomolecule of interest specificity of the detection molecule. Conjugated detection molecules comprising a barcode sequence for their specificity are commercially available (e.g. from Biogen) and may be used in conjunction with the present invention.

After adding and incubating the one or more types of detection molecules with the capture matrix, a washing step may be performed (e.g. with PBS) to remove unbound detection molecules.

The capture matrix with the bound biomolecules of interest may also be transferred to a separate device prior to adding the detection molecules.

One or more sequence elements may be added to the barcode label, such as a barcode sequence B_{T}, an UMI sequence for quantification, and/or a barcode sequence B_{P}, and/or an adapter sequence (AS) for a sequencing platform. As disclosed herein, numerous embodiments exist to introduce these sequence elements and to thereby generate a sequenceable reaction product that comprises one or more of these additional sequence elements.

Optionally, Adding an Oligonucleotide

As was described e.g. in detail in conjunction with the above Figures, after addition of the one or more types of detection molecules and optionally washing the capture matrix, an oligonucleotide may be added to extend the barcode label. Suitable embodiments for the oligonucleotide are described in detail elsewhere herein. Such oligonucleotide may comprise e.g. a barcode sequence B_{T}, an UMI sequence and/or a barcode sequence B_{P}.

In one embodiment, the oligonucleotide is capable of hybridizing to the barcode label (also referred to herein as adaptor barcode oligonucleotide). To allow primer extension, the required reagents (e.g. polymerase, dNTPs etc.) may be added after the oligonucleotide was hybridized or the reagents may added, e.g. perfused, into the compartment, together with the oligonucleotide in case a microfabricated device as described herein is used. Conditions are provided to allow extension of the barcode label using the oligonucleotide as template, whereby an extended barcode label is obtained. The polymerase extension reaction can be conducted within in the compartment. Alternatively, the capture matrix can be transported to another position (compartment) of the cell culture device, or a different device, before performing the polymerase extension reaction.

In an alternative, however less preferred embodiment, the oligonucleotide may be ligated to the barcode label to provide an extended barcode label. Suitable reaction conditions are provided (e.g. ligase, ligase buffer) to allow ligation.

Transfer and Collection of the Capture Matrix

The capture matrix (present at a particular position of the cell culture device, e.g. a particular position of an array of positions; and transferred at a pre-defined time point t) comprising the binding complexes of the one or more types of capture molecules, one or more bound biomolecules of interest, and the bound detection molecules may be removed and transferred to a different compartment (position (m, n) being the position of the (preferably microfabricated) compartment, in which the capture matrix (and the corresponding cell-laden matrix) has/have been incubated, t, being the time point at which the capture matrix was removed from the (microfabricated) compartment and transferred e.g. into another format). Therefore, a reverse flow cherry picking mechanism may be used as described in the disclosure of PCT/EP2018/074526, which is herein incorporated by reference, to transfer the capture matrix to a pre-defined collection position (wherein the position information (e.g. compartment position (m, n)) may be maintained by the particular collection position, wherein the collection position for different compartment positions (m, n) may be different) at a pre-defined time point tₓ The collection position may e.g. be the well of another format such as a 1536 well plate. The cell-laden matrix can remain in its original position (e.g. inside the microfabricated compartment). According to a particular example, the cell-laden matrix may be trapped in its original position by a microfabricated geometry for matrix immobilization. Also the capture matrix may be trapped by such said microfabricated geometry. The removal of the capture matrix may advantageously be achieved by selectively changing the direction and amount of a fluid by a valve arrangement (also referred to as RFCP mechanism). Such a valve arrangement is described above and the disclosure also applies here. Furthermore such a valve arrangement is disclosed in PCT/EP2018/074526, which is herein incorporated by reference. Such a procedure can be advantageously performed according to the present disclosure, in particular in conjunction with the preferred microfabricated cell culture device.

After removal of the capture matrix from the proximity of the cell-laden matrix (e.g. removal of the capture matrix from the microfabricated geometry for matrix immobilization), another capture matrix may be added (e.g. to the free position of the microfabricated geometry for matrix immobilization next to the cell-laden matrix). This can be advantageously achieved by the valve arrangement disclosed above (also referred to as the RFCP mechanism). The capture matrix may be added directly or after a predetermined time interval. Hence, the steps described above, starting with the matrix positioning in proximity to the cell-laden matrix may be repeated one or more times. Thereby, information about the released biomolecules of interest at the different time-points is collected and provided in form of a sequenceable reaction product. The method allows to generate a time-resolved profile of released biomolecules of interest.

Amplification Reaction

After collecting the desired number of capture matrices from one or more time points or one time-point and numerous positions, an amplification reaction is preferably performed to generate multiple copies of the optionally extended barcode label. As is described herein, one or more sequence elements may be added with the primer or the primer combination that is used for amplification, such as a barcode sequence B_{P}, a barcode sequence B_{T}, and/or an adapter sequence (AS) for a sequencing platform. If an UMI sequence is used for quantification, it is introduced prior to amplification. According to one embodiment, a forward primer (e.g. oligonucleotide P-fwd) and a reverse primer (e.g. oligonucleotide T-rev) is used. As is disclosed herein, one or both of the primers of such primer pair may comprise one or more of the sequence elements B_{P}, B_{T}, and/or AS.

The amplification may be a polymerase extension reaction with a single primer (performing repeated cycles of primer extension) or a PCR reaction using a primer pair.

The amplification reaction using one or more (optionally extended) barcode labels as template is preferably performed within a collection well of a device, such as a well-plate. A LightCycler® 1536 Multiwell Plate and a LightCycler® 1536 Instrument from LifeScience may be e.g. used. As is disclosed herein, an amplification reaction may be e.g. performed in a single collection well using as template the (optionally extended) barcode labels from
a capture matrix obtained at a single time point from at least one cell-laden matrix located in a single compartment;
a plurality of capture matrices obtained at two or more time points from at least one cell-laden matrix located in a single compartment, wherein preferably the barcode B_{T} is introduced into the (optionally extended) barcode label prior to performing the amplification reaction;
a plurality of capture matrices obtained from a plurality of cell-laden matrices located in a plurality of different compartments at one or more time points, wherein preferably the barcode B_{P} is introduced into the (optionally extended) barcode label prior to performing the amplification reaction. If the capture matrices were obtained at two or more time points, it is furthermore preferred to also introduce the barcode B_{T} into the (optionally extended) barcode label prior to performing the amplification reaction.

Pooling and Sequencing

After the amplification reaction within each collection position, an aliquot of the generated sequenceable reaction products can be taken from each collection position (e.g. well) and various aliquots may be pooled within a reaction tube. Pooling is possible, as the sequence elements comprised in the sequenceable reaction products allows to identify and correlate each sequenced reaction product e.g. to the original cell-laden matrix and/or time point. The concentration of the pooled sample may be determined (e.g. by using a UV-Vis Spectrophotometer) and adjusted to be compatible with current sequencing procedures. Afterwards the adapted and pooled sample can be sequenced (e.g. by NGS).

Sequencing Analysis

The sequencing process will provide the sequencing data for each barcode label within the generated sequenceable reaction products (e.g. barcode library). The sample containing the pooled aliquots from all collection positions contains different barcode labels comprising the specificity information, as well as e.g. the time information, the position information (n|m), as well as the quantity information indicated by a unique molecular identifier. Thus, based on the sequencing data, an analysis algorithm can be employed to extract the mentioned information and to determine the concentration of the biomolecule of interest. In one embodiment, the following algorithm is used:
1. Identify all barcode sequence that indicated the information about the position of the compartment (position (n, m))
2. From said barcode sequence of step 1, identify all barcode sequence that comprises the barcode sequence indicating the time information (e.g. for different time points t₁, t₂, . . . tₓ)
3. From the previously identified barcode sequence of step 2, identify all barcode sequences comprising the barcode sequence indicating the specificity of the detection molecule and thus the biomolecule of interest (B_{S1}, B_{S2}, . . . B_{Sz}) to be analysed (e.g. TNF-alpha or II-6)
4. From the identified barcode sequence of step 3, count the number of UMIs that are present. This number represents the final concentration (i.e. number) of detected detection molecules at a certain time point. It is assumed, that the binding affinity of the used detection molecules is such that this number is equal to the number of bound biomolecules of interest bound to the capture molecules. Thus with step 4, the concentration of the biomolecule of interest at a certain time point (at a certain position) can be determined.

Steps 1 to 4 may be repeated if required until the concentration of all biomolecules of interest for all time points for all positions is determined. An illustration of the corresponding data gained with the disclosed method is shown in a more general form in FIG. 4.

### II. Example 1

According to Example 1, a method is provided for acquiring a time-resolved profile of one or more biomolecule of interest released by single or multiple cells that are provided in a matrix, preferably a three-dimensional hydrogel matrix. An overview of the process steps of Example 1 is illustrated in FIG. 8. The sequenceable reaction product of the method may be the one depicted in FIG. 3E.

According to Example 1, one or more types of detection molecules are provided comprising a barcode label comprising following sequence elements:
specificity information (B_{S}),
primer sequence (1),
adaptor sequence (1), and
a cleavable linker are associated during or after production of the detection molecule (e.g. commercially available antibodies).

Furthermore, barcode sequences for quantity-, time- and position-information can be added within a collection position (e.g collection well). Sample preparation and handling of capture matrices is performed on a cell culture device, preferably a microfabricated cell culture device. Said microfabricated cell culture device important to the present disclosure as it allows to combine different sequence elements within one oligonucleotide that can be sequenced.

The method according to example 1 comprises the steps described above in the section about the general method steps. Example 1 differs in comparison to the general method steps in following steps:

Adding the One or More Types of Detection Molecules

Adding the one or more types of detection molecules as described above, wherein the detection molecule comprises a barcode label comprising the following elements:
a photo-cleavable linker;
a primer sequence (1) for performing a polymerase chain reaction;
a barcode sequence B_{S} indicating the specificity of the detection molecule;
an adaptor sequence (1).

Optionally, Adding an Oligonucleotide to the Capture Matrix in the Compartment

This step is not performed in Example 1.

Addition of Information to the Barcode Label

After collecting the desired number of capture matrices from one or more time points or one time-point and positions, the quantity- (UMI) time- and position-information can be added to the collected detection molecules, in particular the barcode label:

An oligonucleotide containing an UMI sequence is added to the barcode label encoding the detection molecule specificity by using established methods from molecular biology well known by the person skilled in the art. For example, a polymerization extension reaction or a ligation reaction can be used to transfer the information of oligonucleotide to the barcode label. Afterwards, the barcode sequence indicating the time information and the barcode sequence indicating the position information can be added by using a PCR reaction. An illustration of the process is depicted in FIG. 8a. The addition of the quantity information (UMI) within the collection position (e.g. well) has the advantage that the number of different UMI sequences required for labeling the detection molecules can be significantly reduced. For example, if one capture matrix can be occupied by a total number of 1 million detection molecules, the UMI length does not need to be larger than 10 bp (this corresponds to a total number of 1048576=410 different UMI sequences).

A polymerase extension/elongation and/or amplification reaction within each collection position (e.g. using a LightCycler® 1536 Multiwell Plate and a LightCycler® 1536 Instrument from LifeScience) whereas the forward primer (oligonucleotide P-fwd) contains a barcode sequence indicating the position information (Bₚ) and the reverse primer (e.g. oligonucleotide T-rev) contains a barcode sequence representing the time information (B_{T}) or vice versa to generate an exemplary barcode label (e.g. Oligo-P-Ab-U-T) comprising:
a. the barcode sequence provided by the detection molecule (see above)
b. a unique molecular identifier (UMI)
c. a barcode indicating time information (B_{T})
d. a barcode indicating position information for the compartment (B_{P})
e. optionally, two sequences complementary to commercially available sequencing primers and adaptors from sequencing companies such as 10× Genomics, Oxford Nanopore, Pacific Biosciences, QIAGEN, Agilent Technologies and Illumina.

In one embodiment, each well of an exemplary 1536 well plate contains one unique primer combination (e.g. pair of reverse and forward primer). For example, the well A1 contains a reverse primer that comprises a barcode sequence B_{T} for the time-point tⱼ and a forward primer that comprises a barcode sequence B_{P1} for indicating the position of the compartment (at position (m, n)₁) from which the capture matrix was released (position information). The well A2 might contain a reverse primer that comprises a barcode sequence B_{T2} for the time-point t₂ and a forward primer that comprises a barcode sequence B_{P1} for identifying the position (m, n)₁. The well B1 might contain a reverse primer that comprises a barcode sequence B_{T1} for the time-point t, and a forward primer that comprises a barcode sequence B_{P2} for indicating the position (m, n)₂. Thus, for/positions and x time points the needed number of different primers is: nₚᵣᵢₘₑᵣ=I*x. This number corresponds to the number of required wells n_{wells}=nₚᵣᵢₘₑᵣ. In one advantageous embodiment, the wells are pre-loaded with lyophilized components necessary for performing the PCR (e.g. by using hot-start PCR) prior to the addition of a capture matrices.

The described above embodiment has several advantages: First, it enables the analysis of biomolecules that have been released from single cells, cell pairs and/or small cell colonies located within a 3D microenvironment in a dynamic, time-lapse manner. Second, due to the removal of the capture matrix containing the bound biomolecules of interest, the dynamic range of the detection system is large. For example, if only one capture matrix is used for the whole culture time, the capture molecules might be saturated with released biomolecules of interest within minutes to hours resulting in a limited dynamic measurement range. By using multiple capture matrices capturing only the biomolecules of interest released within a defined period, the dynamic range is increased. Third, the reduction of the reaction volume increases significantly the sensitivity of the detection mechanism as the concentration of the biomolecule of interest is higher due to the small volume reduction. Because barcode labels conjugated to detection molecules (preferably antibodies) permit a nearly unlimited number of molecular targets, analytical multiplexing capability is nearly unlimited.

In addition, the disclosed method offers the following advantages:
The method can be adapted for the detection of any biomolecule of interest, in particular protein, for which a corresponding binding molecule (i.e. detection molecules) such as an antibody is available
The method provides exponential signal amplification due to the use of a polymerase chain reaction (PCR) or polymerase extension reaction which theoretically enables detection of single molecules
Extremely low limit of detection (pg−fg)
Suitable for small sample volumes, in particular for handling of a single cell
Compatible with complex samples
Fewer incubation steps than an ELISA, improved assay reproducibility
Rapid time to results for whole secretome profiles
Wider dynamic range than an ELISA
Highly capable of multiplexing

### III. Example 2

An overview of the process steps of Example 2 is illustrated in FIG. 6. The sequenceable reaction product of the method may be the one depicted in FIG. 3C.

According to Example 2, one or more types of detection molecules are provided comprising a barcode label comprising following information:
a barcode sequence (B_{S}),
a barcode sequences for quantity information (UMI),
and the cleavable linker are added during antibody production (commercially available)

Oligonucleotide sequences for time information and position information are added within a collection well. Antigen binding, washing and handling of capture matrices is performed on a microfabricated cell culture device. Said microfabricated cell culture device is advantageous, as it enables to combine all different information in one oligonucleotide.

The method according to Example 2 comprises the steps described above in the section about the general method steps. Example 2 differs in comparison to the general method steps in following steps:

Adding the One or More Types of Detection Molecules

Adding the one or more types of detection molecules as described above, wherein the detection molecule comprises a barcode label comprising the following elements:
a photo-cleavable linker;
a primer sequence (1) for performing a polymerase chain reaction;
a barcode sequence B_{S} indicating the specificity of the detection molecule;
a barcode sequences for quantity information (UMI); and
a primer sequence (2) for performing a polymerase chain reaction.

According to Example 2, the quantity information (UMI sequence) is part of the barcode label bound to the one or more types of detection molecules. To this end, the capture matrix containing one or more types of capture molecules, bound biomolecules of interest and one or more types of detection molecules labeled with barcode labels encode the detection molecules specificity as well as a UMI sequence is transferred into a collection position (e.g. well).

Conjugated detection molecules having a barcode sequence for their specificity are commercially available (e.g. from Biogen) and can be easily modified with UMI sequences by a skilled person of the art to add the mentioned elements. Degenerate synthesis of oligonucleotides might be used for UMI synthesis.

Optionally, Adding an Oligonucleotide to the Capture Matrix in the Compartment

This step is not performed in Example 2.

Addition of Information to the Barcode Label

A PCR reaction within each collection well (e.g. using a LightCycler® 1536 Multiwell Plate and a LightCycler® 1536 Instrument from LifeScience) using a primer combination is performed, wherein the forward primer (oligonucleotide P-fwd) contains a barcode sequence representing the position information (Bp) and the reverse primer (oligonucleotide T-rev) contains a barcode sequence representing the time information (BT) or vice versa to generate an exemplary sequenceable reaction product (e.g. Oligo-P-Ab-U-T) comprising:
a) the barcode label provided by the one or more types of detection molecules (e.g. Oligo-Ab-U),
b) a time-point specific nucleotide sequence (B_{T}),
c) a position specific nucleotide sequence (B_{P}),
d) adapter sequences (e.g. two sequences complementary to commercially available sequencing primers and adaptors from sequencing companies such as 10× Genomics, Oxford Nanopore, Pacific Biosciences, QIAGEN, Agilent Technologies and Illumina.

The direct incorporation of the UMI into the detection molecule conjugated barcode label eliminates the need for a primer elongation by reverse transcriptase reactions.

### IV. Example 3

An overview of the process steps of Example 3 is illustrated in FIG. 7. The sequenceable reaction product of the method may be the one depicted in FIG. 3D.

According to Example 3, one or more types of detection molecules are provided comprising a barcode label comprising following information:
a barcode sequence (B_{S}),
a barcode sequences for quantity information (UMI),

Time information is added within compartment of the cell culture device containing a capture matrix and cell-laden matrix. Position information is added within the collection well. Sample preparation and handling of the capture matrix is performed utilizing a microfabricated cell culture device. Said microfabricated cell culture device is advantageous as it enables to combine all different information within one oligonucleotide that can be sequenced.

The method according to Example 3 comprises the steps described above in the section about the general method steps. Example 3 differs in comparison to the general method steps in following steps

Adding the One or More Types of Detection Molecules

The addition of one or more types of detection molecules is performed as described in Example 2.

Optionally, Adding an Oligonucleotide to the Capture Matrix in the Compartment

The addition of time information is done by performing an extension of the barcode label bound to the one or more types of detection molecules within the compartment of the cell culture device. After the incubation step and binding, of the biomolecules of interest the compartment containing the capture matrix as well as the cell-laden matrix is perfused with a solution that contains an oligonucleotide with the following elements:
a. a barcode sequence B_{T}, indicating a time information (e.g. time-point specific sequence)
b. an adapter sequence (1) for binding to the barcode label
c. a reverse primer binding sequence (2)_{R}

In one embodiment, the solution containing the oligonucleotide might be a hybridization buffer. Due to the perfusion with the hybridization solution, the oligonucleotide binds to the barcode label (that is coupled to the one or more types of detection molecules) via the adaptor sequence (1). Afterwards, unbound oligonucleotides are washed away by perfusion with washing buffer (e.g. PBS). In a next step, the matrices are perfused with a solution containing a DNA-Polymerase such as IsoPol™ DNA Polymerase (ArcticZymes). Thus, the oligonucleotide is extended and the sequence is added to the barcode label (generating and extended barcode label). The extended barcode label contains now the following elements:
a) a photo-cleavable linker,
b) a primer for a polymerase chain reaction (primer sequence (1)),
c) a barcode sequence B_{S} indicating the specificity of the detection molecule (e.g. an antigen specific sequence (B_{S})),
d) a unique molecular identifier (UMI),
e) an adaptor sequence (1),
f) a barcode sequence B_{T} indicating a time information (e.g. a time-point specific sequence), and
g) a primer sequence (2).

Addition of Information to the Barcode Label

Transferring the capture matrix from the compartment (position (m, n)) that contains the one or more types of capture molecules, bound biomolecules of interest and the barcoded one or more types of detection molecule to a pre-defined well (corresponding well to position (m, n)) of another format such as a 1536 well plate. In a preferred embodiment, this is done using the reverse flow cherry picking mechanism as disclosed. At this step, the detection molecules have coupled an extended barcode label that contains the quantity information, the specificity information as well as the time information. The matrix containing the cell(s) remains within its position. As the time information is added when the capture matrix is still positioned within said compartment of the cell culture device, the number of needed wells for generating the sequenceable reaction product is reduced from n_{well}=I*x to n_{well}=I.

After collecting all detection beads from different time points and positions, the position information is added to the collected extended barcode labels that are coupled to one or more types of detection molecules. For example, this is be done by performing a PCR reaction within each collection well whereas the forward and/or reverse primer (here primer combination) might contain a barcode representing the position information.

### V. Example 4

An overview of the process steps of Example 4 is illustrated in FIG. 9. The sequenceable reaction product of the method may be the one depicted in FIG. 3F.

According to Example 4, one or more types of detection molecules are provided comprising a barcode label comprising following information:

Antigen-specificity information (B_{S}).

Time and quantity information (UMI) is added within compartment of the cell culture device containing amplification matrix and cell-laden matrix.

In another advantageous embodiment, the time information as well as the quantity information is added to the barcode label bound to the one or more type of detection molecules within the compartment. To this end a oligonucleotide contains a barcode sequence indicating a time information B_{T} as well as a quantity information (UMI). An advantage is the reduced UMI library size due to combination of UMIs with B_{T}.

Apart from the difference above, the method according to Example 4 comprises the steps described above in the section about the general method steps.

### VI. Example 5

An overview of the process steps of Example 5 is illustrated in FIG. 5. The sequenceable reaction product of the method may be the one depicted in FIG. 3B.

According to Example 5, one or more types of detection molecules are provided comprising a barcode label comprising following information:
Antigen-specificity (B_{S})/Time(B_{T})/Quantity (UMI) information is added during antibody production

In another advantageous embodiment, the barcode label bound to the one or more types of detection molecule contains the specificity, the quantity and the time information thereby reducing the number of processing steps. Thus, after incubating the capture matrix and the cell-laden matrix (or matrices) and subsequent washing, the capture matrices are perfused with a solution containing one or more types of detection molecules that are labeled with the barcode label containing the specificity, quantity and time information. The capture matrices are finally transferred to a collection well where the position information is added for example by using a PCR.

Apart from the difference above, the method according to Example 5 comprises the steps described above in the section about the general method steps.

### VII. Example 6

The core process steps of Example 6 are illustrated in FIG. 21. The cell-laden matrix is incubated in a cell culture plate as device. The method comprises providing (e.g. generating) a cell-laden matrix, so that it is located in a compartment (e.g. well) of a cell culture plate, e.g. 96 well plate. The cell-laden matrix is positioned in a way in the compartment that the surrounding liquid(s) can be exchanged without affecting the cell-laden matrix. Cells may be encapsulated in a hydrogel plug or hemi-spheres by using a conventional pipette to provide cells positioned within a well plate. Afterwards, the following method steps are performed:
Providing a capture matrix (e.g. by preparing or obtaining as disclosed herein), which comprises one or more types of capture molecules, wherein each type of capture molecule binds a biomolecule of interest;
Incubating the cell-laden matrix to allow release of the one or more biomolecules of interest. As disclosed elsewhere herein, there are different options to bring the capture matrix into contact with the released biomolecule(s) of interest. The capture matrix may e.g. be present prior to or during incubation for release of the biomolecule(s) of interest or the capture matrix may be added after incubation. Addition of the capture matrix after incubation (e.g. for a pre-determined period of time) allows accumulation of the released biomolecule(s) of interest in the surrounding liquid. According to one embodiment, the capture matrix (such as a plurality of capture beads as shown in FIG. 21) is added to the compartment comprising the cell-laden matrix and the surrounding liquid after an incubation period. The one or more biomolecules of interest are allowed to bind to the one or more types of capture molecules of the capture matrix;
   optionally, the method comprises transferring the capture matrix to another location e.g. a different well of the same cell culture plate or to a different cell culture plate for further processing;
Adding one or more types of detection molecules to the capture matrix, wherein each type of detection molecule specifically binds a biomolecule of interest, and wherein each type of detection molecule comprises a barcode label which comprises a barcode sequence (BS) indicating the specificity of the detection molecule (see step c));
   preferably complete removal of unbound detection molecules e.g. by vigorous washing;
Generating a sequenceable reaction product (see step d)) which comprises at least
   the barcode sequence (B_{S}), and
   a barcode sequence (B_{T}) for indicating a time information, and/or
   a barcode sequence (B_{P}) for indicating a position information, and
   optionally a unique molecular identifier (UMI) sequence,
wherein generation of the sequenceable reaction product preferably comprises the use of at least one oligonucleotide, optionally a primer, that is capable of hybridizing to the barcode label of the at least one type of detection molecule; and
preferably, sequencing the generated reaction product (see step e)).

It is noted that the incubation period may be selected by the skilled person in view of the cells comprised in the cell-laden matrix and the biomolecule(s) of interest. In embodiments, the incubation period is selected from the range of 1 h to 72 h, such as 4 h to 72 h. A shorter incubation period (e.g. 1 h to 24 h) may be selected for microbiological applications. For instance, a shorter incubation period may be selected for a prokaryotic cell, such as a bacterial cell, which can be comprised in the cell-laden matrix as disclosed herein. A longer incubation period (e.g. 4 h to 72 h) may be selected for other applications. For instance, a longer incubation period may be selected for a eukaryotic cell, such an animal cell, which can be comprised in the cell-laden matrix.

The method may also comprises one or more cycles of incubation of the cell-laden matrix to allow release on the one or more biomolecules of interest and capture matrix addition in each cycle as discussed above. The repeated incubation and binding can be performed multiple times, e.g. ≥two times, ≥three times, ≥four times, or ≥five times. Suitable time intervals between cycles can be selected by the skilled person. In embodiments, the time interval between cycles is selected from ≥10 min, ≥20 min, ≥30 min, 1 h, ≥2 h, ≥3 h, ≥4 h, 5 h or more, up to days 1 d, 2 d or several days, preferably selected from the range of 30-130 min.

1. A method for analyzing one or more cell released biomolecules, comprising providing a cell-laden matrix, wherein the cell-laden matrix comprises at least one cell that releases one or more biomolecules of interest, wherein the method comprises the following steps:
a) providing a capture matrix, wherein the capture matrix comprises one or more types of capture molecules, wherein each type of capture molecule binds a biomolecule of interest;
b) incubating the cell-laden matrix to allow release of the one or more biomolecules of interest and binding the one or more biomolecules of interest to the one or more types of capture molecules of the capture matrix;
c) adding one or more types of detection molecules, wherein each type of detection molecule specifically binds a biomolecule of interest, and wherein each type of detection molecule comprises a barcode label which comprises a barcode sequence (B_{S}) indicating the specificity of the detection molecule;
d) generating a sequenceable reaction product which comprises at least
(i) the barcode sequence (B_{S}), and
(ii) a barcode sequence (B_{T}) for indicating a time information, and/or
(iii) a barcode sequence (B_{P}) for indicating a position information, and
(iv) optionally a unique molecular identifier (UMI) sequence,
wherein generation of the sequenceable reaction product comprises
the use of at least one oligonucleotide, optionally a primer, that is capable of hybridizing to the barcode label of the at least one type of detection molecule or
the use of at least one oligonucleotide that is ligated to the barcode label of the at least one type of detection molecule.

2. The method according to claim 1, wherein the sequenceable reaction product comprises a barcode sequence (B_{T}) for indicating a time information and wherein n cycles of steps a) to c) and optionally step d) are performed at different time points tₓ, wherein n is at least 2 and x indicates the different time points, and wherein for each cycle a sequenceable reaction product is generated that differs in its barcode sequence B_{T} from the barcode sequence B_{T} of all other performed cycles.

3. The method according to claim 1 or 2, wherein a plurality of cell-laden matrices and capture matrices are provided in a cell culture device comprising a plurality of compartments, wherein at least one cell-laden matrix and at least one capture matrix are provided within a compartment of the cell culture device.

4. The method according to claim 3, wherein the method comprises obtaining capture matrices from a plurality of compartments and transfer of the capture matrices to a device comprising a plurality of compartments.

5. The method according to claim 3 or 4, wherein at least one cycle of steps a) to d) is performed for a plurality of cell-laden matrices comprised in different compartments and wherein the sequenceable reaction product that is generated in step d) comprises a barcode sequence B_{P} for indicating position information of a cell-laden matrix analysed,
wherein a sequenceable reaction product is generated for a cell-laden matrix comprised in a compartment that differs in its barcode sequence B_{P} from the barcode sequence B_{P} of the sequenceable reaction product(s) generated for a cell-laden matrix comprised in another compartment.

6. The method according to claim 5, wherein the barcode sequence B_{P} is introduced into the sequenceable reaction product via an oligonucleotide that is used in step d), wherein the oligonucleotide comprising the barcode sequence B_{P} is a primer that is used in an amplification reaction.

7. The method according to one or more of claims 1 to 6, comprising analyzing y different biomolecules of interest using different types of capture molecules and different types of detection molecules, wherein y is at least 2 and wherein the barcode label of each type of detection molecule that binds a biomolecule of interest differs in its barcode sequence B_{S} from the barcode sequence B_{S} of all other types of detection molecules that bind a different biomolecule of interest.

8. The method according to one or more of claims 1 to 7, wherein step d) comprises performing an amplification reaction using a primer or primer combination, optionally wherein step d) additionally comprises extending the barcode label using an adaptor barcode oligonucleotide capable of hybridizing to the barcode label as template, whereby an extended barcode label is provided in advance of the amplification reaction.

9. The method according to one or more of claims 1 to 8, wherein the method comprises e) sequencing the generated sequenceable reaction product(s), optionally wherein the method comprises pooling sequenceable reaction products generated in step d) from different cycles and/or generated from different compartments and sequencing the obtained pool.

10. The method according to one or more of claims 1 to 9, wherein step d) comprises
(aa) hybridizing at least one oligonucleotide to the barcode label of at least one type of detection molecule and extending said barcode label using the hybridized oligonucleotide as template thereby obtaining an extended barcode label attached to the detection molecule that additionally comprises sequence information of the hybridized oligonucleotide that was used as template,
optionally wherein step d) further comprises
(bb) performing an amplification reaction with a primer or primer combination using the extended barcode label and/or the reverse complement thereof as template, wherein preferably, the extended barcode label is used as template.

11. The method according to one or more of claims 1 to 10, wherein generation of the sequenceable reaction product in step d) comprises the use of
(i) at least one oligonucleotide, optionally a primer, and/or
(ii) a primer combination,
wherein the at least one oligonucleotide and/or the primer combination includes one or more sequence elements selected from the group consisting of
a barcode sequence (B_{T}) for indicating a time information,
a barcode sequence (B_{P}) for indicating position information of a cell-laden matrix,
a unique molecular identifier (UMI) sequence, optionally wherein the UMI sequence has a length of up to 40 nucleotides, preferably 4-20 nucleotides, and
an adapter sequence (AS) for sequencing,
wherein the one or more sequence elements B_{T}, B_{P}, UMI and/or AS, if included, are located 5′ of the sequence region of the oligonucleotide and/or primer that is capable of hybridizing to the barcode label of the detection molecule or the reverse complement thereof.

12. The method according to one or more of claims 1 to 11, wherein the barcode label attached to a detection molecule and/or the extended barcode label obtained according the method of claim 9 to 11 comprises
(i) the barcode sequence (B_{S}) indicating the specificity of the detection molecule;
(ii) one or more primer target sequences;
(iii) optionally a barcode sequence (B_{T}) indicating a time information;
(iv) optionally a unique molecular identifier (UMI) sequence; and
(v) optionally an adapter sequence (1).

13. The method according to one or more of claims 1 to 12, wherein step d) comprises
per Variant A
(aa) adding an adaptor barcode oligonucleotide capable of hybridizing to the barcode label of at least one type of detection molecule, wherein the adaptor barcode oligonucleotide comprises 5′ to the region that is capable of hybridizing to the barcode label a unique molecular identifier (UMI) sequence, and extending the barcode label using the hybridized adaptor barcode oligonucleotide as template thereby obtaining an extended barcode label;
wherein preferably step d) further comprises (bb) performing an amplification reaction with a primer or primer combination using the extended barcode label and/or the reverse complement thereof as template;
or
per Variant B
(aa) adding an adaptor barcode oligonucleotide capable of hybridizing to the barcode label of at least one type of detection molecule, wherein the adaptor barcode oligonucleotide comprises 5′ to the region that is capable of hybridizing to the barcode label (i) a barcode sequence (B_{P}) for indicating a position information and (ii) preferably a unique molecular identifier (UMI) sequence, and extending the barcode label using the hybridized adaptor barcode oligonucleotide as template thereby obtaining an extended barcode label;
wherein preferably, step d) further comprises (bb) performing an amplification reaction with a primer or primer combination using the extended barcode label and/or the reverse complement thereof as template;
or
per Variant C
(aa) adding an adaptor barcode oligonucleotide capable of hybridizing to the barcode label of at least one type of detection molecule, wherein the adaptor barcode oligonucleotide comprises 5′ to the region that is capable of hybridizing to the barcode label (i) a barcode sequence (B_{T}) for indicating a time information and/or (ii) a unique molecular identifier (UMI) sequence, and extending the barcode label using the hybridized adaptor barcode oligonucleotide as template thereby obtaining an extended barcode label;
wherein preferably step d) further comprises (bb) performing an amplification reaction with a primer or primer combination using the extended barcode label and/or the reverse complement thereof as template.

14. The method according to any one of claims 1 to 13, wherein step d) comprises
(aa) adding an adaptor barcode oligonucleotide, wherein the adaptor barcode oligonucleotide comprises an adaptor sequence (1)_{R} that is reverse complementary to an adapter sequence (1) of the barcode label of the detection molecule,
wherein the adaptor barcode oligonucleotide additionally comprises at least one, at least two, at least three or all sequence elements selected from the group consisting of
a barcode sequence (B_{T}) for indicating a time information,
a barcode sequence (B_{P}) for indicating a position information,
a unique molecular identifier (UMI) sequence, and
a primer target sequence,
wherein these one or more sequence elements are located 5′ of the adaptor sequence (1)_{R} and extending the barcode label using the hybridized adaptor barcode oligonucleotide as template thereby obtaining an extended barcode label.

15. The method according to claim 9 to 14, wherein step d) comprises performing an amplification reaction with a primer or primer combination comprising
a barcode sequence (B_{P}) for indicating position information,
optionally an adapter sequence (AS) for sequencing,
optionally a barcode sequence (B_{T}) for indicating a time information,
wherein the one or more sequence elements B_{P}, AS, and/or B_{T} if included in the primer or a primer of the primer combination, are located 5′ of the sequence region of the primer that is capable of hybridizing to the optionally extended barcode label or the reverse complement thereof.

16. The method according to claim 15, wherein the templates comprised in different compartments of a device are contacted with a different subtype of the primer or primer combination, wherein the different subtypes of the primer or primer combination differ in their barcode sequence B_{P} that indicates the position information of an individual compartment, wherein preferably, the subtypes of the primer or primer combination are identical except for the barcode sequence B_{P} that is unique for each subtype.

17. The method according to claim 16, wherein the amplification in step d) is performed by contacting the templates comprised in different compartments of a device with different primer combinations, wherein one primer of the primer combination is the same for all templates comprised in different compartments of the device and the other primer of the primer combination differs in the barcode sequence B_{P} that indicates the position information of an individual compartment.

18. The method according to one or more of claims 1 to 17, wherein the barcode sequence B_{T} is provided in the barcode label or the extended barcode label and wherein step d) comprises pooling barcode labels or extended barcode labels provided at different time points and comprising different barcode sequences B_{T} in a compartment prior to performing an amplification reaction.

19. The method according to one or more of claims 1 to 18, having one or more of the following features
a. the matrix comprising at least one cell has one or more of the following characteristics:
(i) the matrix material is provided by a hydrogel;
(ii) the matrix is three-dimensional;
(iii) the matrix is a particle, optionally a hemi-spherical particle or preferably a spherical particle;
(iv) the matrix has a diameter of ≤1000 μm, such as ≤800 μm, ≤600 μm, or ≤400 μm, preferably ≤200 μm, such as 5 μm to 150 μm; and/or
(vi) the matrix has a volume of ≤200 μl, such as ≤100 μl, ≤50 μl, ≤10 μl, ≤1 μl, ≤0.5 μl, ≤300 nl, <200 nl, ≤100 nl, <50 nl or ≤5 nl, preferably 0.05 pl to 2000 pl;
b. the capture matrix comprising the one or more types of capture molecules has one or more of the following characteristics:
(i) it is a polymer matrix, optionally comprising or consisting of polyacrylamide (PMA), polyactic acid (PLA), poly(vinyl alcohol) (PVA), polyethylene glycol (PEG), polyoxazoline (POx), and polystyrene (PS).
(ii) the matrix material is provided by a hydrogel;
(iii) the matrix is three-dimensional;
(iv) the matrix is a particle, preferably a spherical particle; and/or
(v) the matrix has a diameter of ≤1000 μm, such as ≤800 μm, ≤600 μm or ≤400 μm, preferably ≤200 μm, such as 5 μm to 150 μm;
and/or
c. the cell-laden matrix and the capture matrix are provided in proximity within a compartment of a device or the cell-laden matrix and the capture matrix are provided in separate compartments, wherein the separate compartments are in fluid communication with each other or can be brought in fluid communication with each other so that the released biomolecules of interest can contact the capture matrix.

20. The method according to one or more of claims 1 to 19, wherein the matrix of the cell-laden matrix is a hydrogel which has one or more of the following characteristics:
a. the hydrogel comprises cross-linked hydrogel precursor molecules of the same type or of different types;
b. the hydrogel is composed of at least two different polymers with different structures as hydrogel precursor molecules, wherein optionally, at least one polymer is a copolymer;
c. the hydrogel is formed using at least one polymer which has a linear structure and at least one polymer which has a multiarm or star-shaped structure;
d. the hydrogel is formed using a t least one polymer of formula (P1) 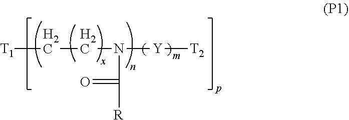
wherein
R is independently selected from a hydrogen atom, a hydrocarbon with 1-18 carbonatoms (preferably CH₃, —C₂H₅,), a C₁-C₂₅-hydrocarbon with at least one hydroxy group, a C₁-C₂₅-hydrocarbon with at least one carboxy group, (C₂-C₆)alkylthiol, (C₂-C₆)alkylamine, protected (C₂-C₆)alkylamine (preferably-(CH₂)₂₋₆—NH—CO—R (with R=tert-Butyl, perfluoroalkyl)), (C₂-C₆)alkylazide, polyethylene glycol, polylactic acid, polyglycolic acid, polyoxazoline, or wherein R is a residue R⁴
Y is a moiety containing at least one graft, comprising at least one residue R⁴,
T₁ is a terminating moiety, which may contain a residue R⁴,
T₂ is a terminating moiety, which contains a residue R⁴,
p is an integer from 1 to 10,
n is an integer greater than 1 and preferably, below 500,
m is zero or an integer of at least, preferably greater than 1, and preferably, below 500,
the sum n+m is greater than 10,
x is independently 1, 2 or 3, preferably x is independently 1 or 2, most preferably x is 1,
R⁴ independently comprise at least one functional group
for crosslinking and/or
for binding biologically active compounds, and
optionally comprising a (preferably degradable) spacer moiety connecting said functional group with the binding site to the respective moiety of the structure of formula (P1),
wherein the entirety of all m-fold and n-fold repeating units are distributed in any order within the polymer chain and wherein optionally, the polymer is a random copolymer or a block copolymer.

21. The method according to one or more of claims 1 to 20, wherein the method is performed by utilizing a cell culture device, which preferably is a microfabricated cell culture device, wherein the device has one or more of the following features:
i) at least one compartment for accommodating at least one, preferably at least two matrices, including at least one capture matrix and/or at least one cell-laden matrix;
ii) at least one compartment that is capable of being switched between an isolated and an open state, wherein the isolated state corresponds to a state at which fluid that is present in the compartment is in no contact with fluid not present in the compartment and wherein the open state corresponds to a state at which fluid that is present in the compartment is in contact with fluid not present in the compartment;
iii) a compartment for accommodating at least one matrix, preferably two matrices, wherein a microfabricated geometry for matrix immobilization is present suitable for positioning the at least one matrix;
iv) a plurality of compartments for accommodating at least one matrix, preferably provided by an array of compartments;
v) a microfabricated valve capable of switching the compartment to an open or closed state;
vi) a microfabricated valve, comprising a first channel, a second channel, a connection channel connecting the first channel and the second channel, a valve portion arranged within the connection channel, wherein the valve portion is adapted to selectively open and close the connection channel;
vii) a microfabricated valve comprising at least three layers, wherein a first channel is located within a first layer; a second channel is located within a third layer; a valve portion is located within a second layer; the second layer is arranged between the first and the third layer;
viii) a microfabricated valve wherein a first channel comprises a microfabricated geometry for matrix immobilization suitable for positioning at least one matrix being contained in a fluid which flows through the first channel, wherein the microfabricated geometry for matrix immobilization is arranged within the first channel in such a way that a fluid flow can be reduced by the microfabricated geometry for matrix immobilization, in particular, the microfabricated geometry for matrix immobilization narrows the cross section of the channel; and/or
wherein a second channel comprises a microfabricated geometry for matrix immobilization suitable for positioning particles being contained in a fluid which flows through the second channel, wherein the microfabricated geometry for matrix immobilization is arranged within the second channel in such a way that a fluid flow can be reduced by the microfabricated geometry for matrix immobilization, in particular, the microfabricated geometry for matrix immobilization narrows the cross section of the channel;
and/or
ix) a fluid reservoir and fluid channels for providing fluid to the compartment.

22. The method according to one or more of claims 1 to 21, wherein the method is performed by utilizing a cell culture device, which preferably is a microfabricated cell culture device, wherein the device comprises one or more of the following features:
i) at least one matrix is releasably positioned by a preferably microfabricated geometry for matrix immobilization inside a compartment;
ii) at least one matrix is releasably positioned by a preferably microfabricated geometry for matrix immobilization inside a compartment, wherein the geometry for matrix immobilization has one or more of the following characteristics:
it is capable of positioning the cell-laden matrix and the capture matrix in proximity;
it is capable of positioning at least two cell-laden matrix and the capture matrix in proximity;
iii) at least one cell-laden matrix and at least one capture matrix are positioned by a preferably microfabricated geometry for matrix immobilization inside a compartment, wherein the compartment accommodating the at least one cell-laden matrix is different from the compartment accommodating the at least one capture matrix and wherein both compartments can be switched to be either in fluid contact with other or to be in no fluid contact with each other;
and/or
iv) it comprises a trapping geometry comprising a valve arrangement adapted to provide a fluid passing through a microfabricated geometry for matrix immobilization wherein the valve arrangement is adapted to selectively change the direction of fluid passing the microfabricated geometry for matrix immobilization, in particular wherein a fluid a first direction urging the at least one matrix into the microfabricated geometry for matrix immobilization and a fluid in the second direction urging the at least one matrix out of the microfabricated geometry for matrix immobilization, and in particular fluid in the second direction delivering the at least one matrix in direction of an exit section.

23. The method according to one or more of claims 1 to 22, wherein the provided cell-laden matrix and capture matrix are provided with a fluid, preferably a fluid that is immiscible with water, wherein said matrices, provided with said fluid, are preferably generated by utilizing a cell culture device, which preferably is a microfabricated cell culture device, and preferably by
(i) releasably positioning the cell-laden matrix and the capture matrix by a preferably microfabricated geometry for matrix immobilization inside a compartment, wherein the compartment comprises a first fluid, preferably an aqueous fluid;
(ii) removing the first fluid from the compartment and replacing the first fluid by a second fluid that provides said fluid, wherein said fluid is preferably immiscible with water; and
(iii) optionally, removing the second fluid from the compartment and replacing it by the first fluid or a third fluid, that is preferably immiscible with the second fluid.

24. The method according to one or more of claims 1 to 23, wherein the cell-laden matrix is incubated to allow release of one or more biomolecules of interest before providing the capture matrix in step a), wherein after providing the capture matrix, one or more biomolecules of interest are specifically bound by the one or more types of capture molecules of the capture matrix;
wherein preferably, the cell-laden matrix is provided in a defined volume of a fluid, preferably a fluid that is immiscible with water, and wherein the capture matrix is provided in a defined volume of the same type of fluid, and wherein after contacting the cell-laden matrix and the capture matrix said fluids of the same type merge to provide a defined volume of fluid that is shared by the cell-laden matrix and the capture matrix.

25. A kit comprising
a) one or more types of detection molecules, wherein each type of detection molecule specifically binds a biomolecule of interest, and wherein each type of detection molecule comprises a barcode label which comprises a barcode sequence (B_{S}) indicating the specificity of the detection molecule; and
b) at least one oligonucleotide, optionally a primer, that is preferably capable of hybridizing to the barcode label of the at least one type of detection molecule.

26. The kit according to claim 25, wherein the oligonucleotide comprises at least one sequence element selected from the group consisting of
(i) a barcode sequence (B_{T}) for indicating a time information,
(ii) a barcode sequence (B_{P}) for indicating a position information, and
(iii) a unique molecular identifier (UMI) sequence.

27. The kit according to claim 25 or 26, wherein the kit has one or more of the following characteristics:
a. it comprises an adaptor barcode oligonucleotide capable of hybridizing to the barcode label of at least one type of detection molecule, wherein the adaptor barcode oligonucleotide comprises 5′ to the region that is capable of hybridizing to the barcode label (i) a barcode sequence (B_{T}) for indicating a time information, a barcode sequence (B_{P}) for indicating a position information, and/or (ii) a unique molecular identifier (UMI) sequence;
b. it comprises an adaptor barcode oligonucleotide, wherein the adaptor barcode oligonucleotide comprises an adaptor sequence (1)_{R} that is reverse complementary to an adapter sequence (1) of the barcode label of the detection molecule, wherein the adaptor barcode oligonucleotide additionally comprises at least one, at least two, at least three or all sequence elements selected from the group consisting of
a barcode sequence (B_{T}) for indicating a time information,
a barcode sequence (B_{P}) for indicating a position information,
a unique molecular identifier (UMI) sequence, and
a primer target sequence,
wherein these one or more sequence elements are located 5′ of the adaptor sequence (1)_{R};
c. a primer or primer combination comprising one or more of the following
a barcode sequence (B_{P}) for indicating position information,
a barcode sequence (B_{T}) for indicating a time information,
an adapter sequence (AS) for sequencing,
wherein the one or more sequence elements B_{P}, AS, and/or B_{T} if included in the primer or a primer of the primer combination, are located 5′ of the sequence region of the primer that is capable of hybridizing to the optionally extended barcode label or the reverse complement thereof;
and/or
d. the barcode label of the one or more types of detection molecules comprises
(i) the barcode sequence (B_{S}) indicating the specificity of the detection molecule;
(ii) one or more primer target sequences;
(iii) optionally a barcode sequence (B_{T}) indicating a time information;
(iv) optionally a unique molecular identifier (UMI) sequence; and
(v) optionally an adapter sequence (1).

28. The kit according to any one of claims 25 to 27, wherein the kit comprises at least one set of oligonucleotides selected from the following group:
a) set 1 comprising:
a. a barcode label attached to the detection molecule comprising:
i. optionally a cleavable linker/spacer,
ii. optionally a first primer binding sequence (1),
iii. a barcode sequence B_{S},
iv. an adaptor sequence (1);
b. an adaptor barcode oligonucleotide comprising:
i. an adaptor sequence (1)_{R},
ii. a unique molecular identifier (UMI) sequence,
iii. a second primer binding sequence (2)_{R},
c. a forward primer comprising:
i. a primer sequence (1),
ii. a barcode sequence B_{P},
iii. an adaptor sequence for sequencing (AS);
d. a reverse primer comprising:
i. a primer sequence (2)_{R},
ii. a barcode sequence B_{T},
iii. an adaptor sequence for sequencing (AS);
b) set 2 comprising:
a. a barcode label attached to the detection molecule comprising:
i. optionally a cleavable linker/spacer,
ii. a first primer binding sequence (1),
iii. a barcode sequence B_{S},
iv. an adaptor sequence (1);
b. an adaptor barcode oligonucleotide comprising:
i. an adaptor sequence (1)_{R},
ii. a barcode sequence B_{P},
iii. a unique molecular identifier (UMI) sequence,
iv. a second primer binding sequence (2)_{R};
c. a forward primer comprising:
i. a primer sequence (1),
ii. a barcode sequence B_{T},
iii. an adaptor sequence for sequencing (AS);
d. a reverse primer comprising:
i. a primer sequence (2)_{R},
ii. an adaptor sequence for sequencing (AS);
c) set 3 comprising:
a. a barcode label attached to the detection molecule comprising:
i. optionally a cleavable linker/spacer,
ii. a first primer binding sequence (1),
iii. a barcode sequence B_{S},
iv. an adaptor sequence (1);
b. an adaptor barcode oligonucleotide comprising:
i. an adaptor sequence (1)_{R},
ii. a barcode sequence B_{T},
iii. a unique molecular identifier (UMI) sequence,
iv. a second primer binding sequence (2)_{R};
c. a forward primer comprising:
i. a primer sequence (1),
ii. a barcode sequence B_{P},
iii. an adaptor sequence for sequencing (AS);
d. a reverse primer comprising:
i. a primer sequence (2)_{R},
ii. an adaptor sequence for sequencing (AS);
d) set 4 comprises:
a. a barcode label attached to the detection molecule comprising:
i. optionally a cleavable linker/spacer,
ii. a first primer binding sequence (1),
iii. a barcode sequence B_{S},
iv. a unique molecular identifier (UMI) sequence,
v. a barcode sequence B_{T},
vi. a second primer binding sequence (2);
b. a forward primer comprising:
i. a primer sequence (1),
ii. a barcode sequence B_{P},
iii. an adaptor sequence for sequencing (AS);
c. a reverse primer comprising:
i. a primer sequence (2)_{R},
ii. an adaptor sequence for sequencing (AS).

29. The kit according to any one of claims 25 to 28, wherein the kit comprises at least one of the following
a. one or more types of capture molecules, wherein each type of capture molecule binds a biomolecule of interest, wherein preferably, the one or more types of capture molecules provided in the kit bind the same biomolecules of interest as the one or more types of detection molecules comprised in the kit;
b. one or more polymers for providing the matrix for the cells and/or the capture matrix, wherein preferably the polymer is capable of forming a hydrogel;
c. a composition, preferably a solution, containing capture matrices;
d. polymerase and/or dNTPs; and/or
e. a wash solution.

30. The kit according to any one of claims 25 to 29, wherein the kit comprises a device with a plurality of compartments, preferably a multi-well plate, wherein said device has one or more of the following characteristics:
a. compartments of the device comprise an oligonucleotide, preferably an adaptor barcode oligonucleotide and/or a primer or primer combination, as defined in claims 25 to 29;
b. compartments of the device comprise at least one set as defined in claim 28;
c. compartments comprising an oligonucleotide, preferably an adaptor barcode oligonucleotide and/or a primer or primer combination, as defined in any one of claims 25 to 29, furthermore comprise reagents for performing an extension and/or amplification reaction;
and/or
d. the device is selected from a 96, 384 or 1536 well plate.

31. A plurality of sequenceable products, wherein each sequenceable product comprises at least the following sequence elements
(i) a barcode sequence (B_{S}) for indicating a specificity, and
(ii) a barcode sequence (B_{T}) for indicating a time information, and/or
(iii) a barcode sequence (B_{P}) for indicating a position information, and
(iv) optionally a unique molecular identifier (UMI) sequence.

32. The plurality sequenceable products according to claim 31, wherein the sequenceable products differ from each other in one or more of the comprised sequence elements (i) to (iv).

33. The plurality of sequenceable products as defined in claim 31 or 32, having one or more of the following features:
a. the number of sequenceable products comprising different sequence elements B_{S}, B_{T} and/or B_{P} is at least 50, preferably at least 100;
b. the plurality of sequenceable products comprise at least 2 different barcode sequences B_{S}, optionally wherein the number of different barcode sequences B_{S} may lie in a range of 2 to 100, 5 to 50, 5 to 25, 5 to 20 or 7 to 15;
c. the plurality of sequenceable products comprise at least 2 different barcode sequences B_{T}, optionally wherein the number of different barcode sequences B_{T} may lie in a range of 2 to 200, 5 to 50, 5 to 25, 5 to 20 or 7 to 15; and
d. the plurality of sequenceable products comprise at least 2 different barcode sequences B_{P}, optionally wherein the number of different barcode sequences B_{P} may lie in a range of 2 to 1000, 5 to 1000, 10 to 500, 20 to 250 or 50 to 200; and/or
e. wherein the UMI sequence has a length of up to 40 nucleotides, preferably 4 to 20 nucleotides.

34. The method according to one or more of claims 1-20, wherein the cell-laden matrix is provided in a compartment of a cell culture plate such that liquid that covers the cell-laden matrix can be removed or exchanged without affecting the cell-laden matrix, and wherein the cell-laden matrix comprises more than one cell and is provided by a three-dimensional hydrogel matrix, optionally having at least partially an ellipsoidal shape, preferably a plug or semi-sphere shape.

35. The method according to claim 34, wherein one or more capture matrices are provided in step a), the method having one or both of the following characteristics:
incubating the cell-laden matrix to allow release of one or more biomolecules of interest before adding the provided capture matrix/matrices to the compartment of the cell culture plate to bind the one or more released biomolecules of interest to the one or more types of capture molecules of the capture matrix, wherein optionally incubating is performed for an incubation period selected from 1 h to 72 h; and/or
the capture matrix is transferred to another compartment after binding the one or more biomolecules of interest to the one or more types of capture molecules of the capture matrix in step b).

36. The method according to one or more of claims 1-20 or 34-35, wherein biomolecules are analyzed time-dependently, wherein the time interval between analyses is selected from ≥10 min, ≥20 min, ≥30 min, ≥1 h, ≥2 h, ≥3 h, ≥4 h, 5 h or more, up to days 1 d, 2 d or several days, preferably selected from the range of 30-120 min.
